(19) United States
(12) Patent Application Publication
(10) Pub. No.: US 2017/0087134 A1
(43) Pub. Date: Mar. 30, 2017

(54) FORMULATIONS OF RIFAXIMIN AND USES THEREOF

(71) Applicant: Salix Pharmaceuticals, Ltd, Bridgewater, NJ (US)

(72) Inventors: Pam Golden, Durham, NC (US); Mohammed A. Kabir, Cary, NC (US)

(21) Appl. No.: 15/281,543

(22) Filed: Sep. 30, 2016

Related U.S. Application Data
(63) Continuation-in-part of application No. 14/250,293, filed on Apr. 10, 2014.
Application No. 14/250,293 is a continuation of application No. 13/181,481, filed on Jul. 12, 2011, now abandoned.
(60) Provisional application No. 61/419,056, filed on Dec. 2, 2010.
(60) Provisional application No. 61/363,609, filed on Jul. 12, 2010.

(51) Int. Cl. A61K 31/439; A61K 47/38; A61K 47/10; A61K 47/02; A61K 47/12

(52) U.S. Cl. CPC A61K 31/439; A61K 47/02; A61K 47/12; A61K 47/10; A61K 47/38

(57) ABSTRACT

The present invention relates to new rifaximin forms comprising solid dispersions of rifaximin, methods of making same and to their use in medicinal preparations and therapeutic methods.

### RELATED APPLICATIONS

This application is a continuation-in-part of U.S. patent application Ser. No. 14/250,293 filed 10 Apr. 2014 which claims the benefit of U.S. patent application Ser. No. 13/181,481 filed 12 Jul. 2011 which claims the benefit of U.S. Provisional Application No. 61/363,609 filed 12 Jul. 2010, and U.S. Provisional Application No. 61/419,056, filed 2 Dec. 2010, the entire contents of each of which are hereby incorporated herein by reference.

### BACKGROUND

Rifaximin (INN; see The Merck Index, XIII Ed., 8304) is an antibiotic belonging to the rifamycin class of antibiotics, e.g., a pyrido-imidazo rifamycin. Rifaximin exerts its broad antibacterial activity, for example, in the gastrointestinal tract against localized gastrointestinal bacteria that cause infectious diarrhea, irritable bowel syndrome, small intestinal bacterial overgrowth, Crohn's disease, and pancreatic insufficiency among other diseases. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

Rifaximin is described in Italian Patent IT 1154655 and EP 0161534, both of which are incorporated herein by reference in their entirety for all purposes. EP 0161534 discloses a process for rifaximin production using rifamycin O as the starting material (The Merck Index, XIII Ed., 8301). U.S. Pat. No. 7,045,620 B1 and PCT Publication WO 2006/094662 A1 disclose polymorphic forms of rifaximin There is a need in the art for formulations of rifaximin to better treat gastrointestinal and other diseases.

### SUMMARY

Provided herein are solid dispersion forms of rifaximin with a variety of polymers and polymer concentrations.

In one aspect, provided herein are solid dispersion forms of rifaximin.

In one embodiment, the solid dispersion form of rifaximin is characterized by an XRPD substantially similar to one or more of the XRPDs of FIGS. 2, 7, 12, 17, 22, 31, and 36.

In one embodiment, the solid dispersion form of rifaximin is characterized by a Thermogram substantially similar to FIGS. 3-6, 8-11, 13-16, 18-21, 23-26, 27-30, and 32.

In one embodiment, the solid dispersion form has the appearance of a single glass transition temperature (Tg).

In one embodiment, a Tg of a solid dispersion form increases with an increased rifaximin concentration

In one embodiment, a solid dispersion form stressed at 70° C./75% RH for 1 week, solids are still x-ray amorphous according to XRPD.

In one embodiment, a solid dispersion form stressed at 70° C./75% RH for 3 weeks, solids are still x-ray amorphous according to XRPD.

In one embodiment, a solid dispersion form stressed at 70° C./75% RH for 6 weeks, solids are still x-ray amorphous according to XRPD.

In one embodiment, a solid dispersion form stressed at 70° C./75% RH for 12 weeks, solids are still x-ray amorphous according to XRPD.

In one aspect, provided herein are microgranules comprising one or more of the solid dispersion forms of rifaximin described herein.

In one embodiment, the microgranules further comprise a polymer.

In one embodiment, the polymer comprises one or more of polyvinylpyrrolidone (PVP) grade K-90, hydroxypropyl methylcellulose phthalate (HPMC-P) grade 55, hydroxypropyl methylcellulose acetate succinate (HPMC-AS) grades HG and MG, or a polymethacrylate (Eudragit® L100-55).

In specific embodiments, the microgranules comprise 25-75% polymer, 40-60% polymer, or 40-50% polymer. In an exemplary embodiment, the microgranules comprise 42-44% polymer.

In one embodiment, the microgranules comprise equal amounts of rifaximin and polymer.

In the present disclosure, when a numerical value is modified by the term “about”, the exact numerical value is also deemed to be disclosed.

In one embodiment, the solid dispersion form of rifaximin comprises one or more polymers selected from polyvinylpyrrolidone (PVP) grade K-90, hydroxypropyl methylcellulose phthalate (HPMC-P) grade 55, hydroxypropyl methylcellulose acetate succinate (HPMC-AS), and a polymethacrylate such as e.g., Eudragit® L100-55). The amount of polymer in the solid dispersion can vary depending upon the nature and amounts of other components. Typical amounts of the polymers in the solid dispersion are e.g., from about 10 wt % to about 60 wt %, from about 10 wt % to about 50 wt %, from about 10 wt % to about 40 wt % from about 12 wt % to about 38 wt %, from about 15 wt % to about 35 wt %, from about 16 wt % to about 34 wt %, from about 30 wt % to about 40 wt %, from about 30 wt % to about 35 wt %, from about 33 wt % to about 35 wt %, about 32 wt %, about 33 wt %, about 34 wt %, about 35 wt %, from about 10 wt % to about 20 wt %, from about 13 wt % to about 18 wt %, from about 16 wt % to about 18 wt %, about 15 wt %, about 16 wt %, about 17 wt %, about 18 wt %, from about 40 wt % to about 50 wt %, from about 46 wt % to about 49 wt %, about 46 wt %, about 47 wt %, or about 48 wt %. In one aspect, the solid dispersion form of rifaximin comprises from about 46 wt % to about 49 wt %, about 46 wt %, about 47 wt %, about 48 wt %, from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt % HPMC-AS. In another aspect, the solid dispersion form of rifaximin comprises about 46 wt %, about 47 wt %, about 48 wt %, about 33 wt %, about 34 wt %, about 35 wt %, about 16 wt %, about 17 wt %, or about 18 wt % HPMC-AS. In yet another aspect, the solid dispersion form of rifaximin comprises about 46 wt %, about 47 wt %, or about 48 wt % HPMC-AS.

In one aspect, the solid dispersion of rifaximin comprises equal amounts of rifaximin and polymer. For example, exemplary embodiments include solid dispersions of rifaximin comprising from about 10 wt % to about 60 wt %, from about 10 wt % to about 50 wt %, from about 10 wt % to about 40 wt %, from about 12 wt % to about 38 wt %, from about 15 wt % to about 35 wt %, from about 16 wt % to about 34 wt %, from about 30 wt % to about 40 wt %, from about 30 wt % to about 35 wt %, from about 33 wt % to about 35 wt %, about 32 wt %, about 33 wt %, about 34 wt %, about 35 wt %, from about 10 wt % to about 20 wt %, from about 13 wt % to about 18 wt %, from about 16 wt % to about 18 wt %, about 15 wt %, about 16 wt %, about 17 wt %, about 18 wt %, from about 40 wt % to about 50 wt %, from about 46 wt % to about 49 wt %, about 46 wt %, about 47 wt %, or about 48 wt % rifaximin and HPMC-AS. In one aspect, the solid dispersion of rifaximin comprises from about 46 wt % to about 49 wt %, about 46 wt %, about 47 wt %, about 48 wt %, from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt % rifaximin and HMPC-AS. In another aspect, the solid dispersion form of rifaximin comprises about 46 wt %, about 47 wt %, about 48 wt %, about 33 wt %, about 34 wt %, about 35 wt %, about 16 wt %, about 17 wt %, or about 18 wt % rifaximin and HPMC-AS. In yet another aspect, the solid dispersion form of rifaximin comprises about 46 wt %, about 47 wt %, or about 48 wt % rifaximin and HPMC-AS.

In another embodiment, the microgranules further comprise an intragranular release controlling agent. In exemplary embodiments, the intragranular release controlling agent comprises a pharmaceutically acceptable excipient, disintegrant, crosprovidone, sodium starch glycolate, corn starch, microcrystalline cellulose, cellulosic derivatives, sodium bicarbonate, and sodium alginate.

In one embodiment, the intragranular release controlling agent comprises between about 2 wt % to about 40 wt % of the microgranule, about 5 wt % to about 20 wt % of the microgranule, or about 10 wt % of the microgranule.

In another embodiment, the intragranular release controlling agent comprises a pharmaceutically acceptable disintegrant, e.g., one selected from the group consisting of crosprovidone, sodium starch glycolate, corn starch, microcrystalline cellulose, cellulosic derivatives, sodium bicarbonate, and sodium alginate.

In another embodiment, the microgranules further comprise a wetting agent or surfactant, e.g., a non-ionic surfactant.

In one embodiment, the non-ionic surfactant comprises between about 2 wt % to about 10 wt % of the microgranule, between about 4 wt % to about 8 wt % of the microgranule, or about 5.0 wt % of the microgranule.

In one embodiment, the non-ionic surfactant comprises a poloxamer, e.g., poloxamer 407 also known as Pluronic F-127.

In one embodiment, the solid dispersion form of rifaximin further comprises a wetting agent or surfactant, e.g., a non-ionic surfactant. The amount of wetting agent or surfactant in the solid dispersion can vary depending upon the nature and amounts of other components. Typical amounts are e.g., from about 0.5 wt % to about 7 wt %, from about 0.5 wt % to about 5 wt %, from about 1 wt % to about 5 wt %, from about 1 wt % to about 4 wt %, from about 2 wt % to about 4 wt %, from about 4 wt % to about 6 wt %, from about 3 wt % to about 5 wt %, from about 2 wt % to about 4 wt %, from about 1 wt % to about 2 wt %, about 1 wt %, about 2 wt %, about 3 wt %, about 4 wt %, about 5 wt %, about 5.5 wt %, or about 6 wt %. In one aspect, the solid dispersion of rifaximin comprises about 1 wt %, about 2 wt %, about 3 wt %, about 4 wt %, about 5 wt %, about 5.5 wt %, about 5.6 wt %, about 5.7 wt %, or about 6 wt % poloxamer 407 (also known as Pluronic F-127). In another aspect, the solid dispersion of rifaximin comprises about 5 wt %, about 5.5 wt %, or about 6 wt % poloxamer 407.

In another embodiment, the microgranules further comprise an antioxidant. In another embodiment, the solid dispersion of rifaximin further comprises an antioxidant

In exemplary embodiments, the antioxidant is butylated hydroxyanisole (BHA), butylated hydroxytoluene (BHT) or propyl gallate (PG).

In another embodiment, the antioxidant comprises between about 0.1 wt % to about 3 wt % of the microgranule or between about 0.5 wt % to about 1 wt % of the microgranule.

In another aspect, provided herein are pharmaceutical compositions comprising the microgranules described herein.

In another aspect, provided herein are pharmaceutical compositions comprising the solid dispersions of rifaximin described herein.

In one embodiment, the pharmaceutical compositions further comprise one or more pharmaceutically acceptable excipients.

In one embodiment, the pharmaceutical compositions are tablets or capsules.

In one embodiment, the pharmaceutical compositions comprise a disintegrant.

In one embodiment, the polymer comprises one or more of polyvinylpyrrolidone (PVP) grade K-90, hydroxypropyl methylcellulose phthalate (HPMC-P) grade 55, hydroxypropyl methylcellulose acetate succinate (HPMC-AS) grades HG and MG, or a polymethacrylate (Eudragit® L100-55).

In one aspect, provided herein are pharmaceutical solid dispersion formulations comprising: rifaximin, HPMC-AS, at a rifaximin to polymer ratio of 50:50, a non-ionic, surfactant polyol and a intragranular release controlling agent.

In one embodiment, the intragranular release controlling agent comprises about 10 wt % of the formulation.

In one aspect, provided herein are processes for producing a solid dispersion of rifaximin comprising: making a slurry of methanol, rifaximin, a polymer and a surfactant; spray drying the slurry; and blending the spray dried slurry with a intragranular release controlling agent.

In one aspect, provided herein are processes for producing a solid dispersion of rifaximin comprising: making a slurry of methanol, rifaximin, HPMC-AS MG and Pluronic F-127; spray drying the slurry; and blending the spray dried slurry with a intragranular release controlling agent.

In one embodiment, the intragranular release controlling agent comprises croscarmellose sodium.

A process for producing form solid dispersion of rifaximin comprising one or more of the methods listed in Tables 1-5.

In one embodiment, pharmaceutical compositions comprising SD rifaximin, a polymer, a surfactant, and a release controlling agent are provided. In one embodiment, provided are pharmaceutical compositions comprising SD rifaximin, HPMC-AS, pluronic F127, and croscarmellose Na (CS). In one embodiment, the pharmaceutical compositions are tablets or pills.

Although it will be understood that the amount of croscarmellose sodium present in the pharmaceutical composition (e.g., tablet) comprising the solid dispersion form of rifaximin can vary, typical amounts are e.g., from about 2 wt % to about 15 wt %, from about 3 wt % to about 14 wt %, from about 4 wt % to about 14 wt %, from about 2 wt % to about 13 wt %, from about 3 wt % to about 13 wt %, from about 4 wt % to about 13 wt %, from about 11 wt % to about 14 wt %, from about 12 wt % to about 14 wt %, from about 4 wt % to about 10 wt %, about 12 wt %, about 12.5 wt %, about 13 wt %, about 13.5 wt %, from about 4 wt % to about 6 wt %, about 5 wt %, from about 8% to about 10 wt %, or about 9 wt % based on the total amount (wt %) of components in the pharmaceutical composition. In one aspect, the amount of croscarmellose sodium present in the pharmaceutical composition (e.g., tablet) comprising the solid dispersion form of rifaximin is from about 4 wt % to about 14 wt %, from about 12 wt % to about 14 wt %, about 13 wt %, from about 4 wt % to about 6 wt %, about 5 wt %, from about 8% to about 10 wt %, or about 9 wt % based on the total amount (wt %) of components in the pharmaceutical composition. In another aspect, the amount of croscarmellose sodium present in the pharmaceutical composition (e.g., tablet) comprising the solid dispersion from of rifaximin is about 13 wt %, about 5 wt %, or about 9 wt % based on the total amount (wt %) of components in the pharmaceutical composition.

Thus, in one aspect, provided herein are pharmaceutical compositions (e.g., a tablet) comprising i) from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt % rifaximin and HMPC-AS in equal amounts based on the total amount (wt %) of components in the pharmaceutical composition; ii) from about 1 wt % to about 4 wt %, from about 3 wt % to about 5 wt %, from about 2 wt % to about 4 wt %, from about 1 wt % to about 2 wt %, about 1 wt %, about 2 wt %, about 3 wt %, or about 4 wt % poloxamer 407 based on the total amount (wt %) of components in the pharmaceutical composition; and iii) from about 4 wt % to about 14 wt %, from about 12 wt % to about 14 wt %, about 13 wt %, from about 4 wt % to about 6 wt %, about 5 wt %, from about 4 wt % to about 10 wt %, from about 8% to about 10 wt %, or about 9 wt % croscarmellose sodium based on the total amount (wt %) of components in the pharmaceutical composition.

In another aspect, provided herein are pharmaceutical compositions (e.g., a tablet) comprising i) about 33 wt %, about 34 wt %, about 35 wt %, from about 16 wt % to about 18 wt %, or about 17 wt % rifaximin and HMPC-AS in equal amounts based on the total amount (wt %) of components in the pharmaceutical composition; ii) about 1 wt %, about 2 wt %, or about 4 wt % poloxamer 407 based on the total amount (wt %) of components in the pharmaceutical composition; and iii) about 13 wt %, about 5 wt %, or about 9 wt % croscarmellose sodium based on the total amount (wt %) of components in the pharmaceutical composition.

In additional embodiments, the pharmaceutical compositions further comprise one or more fillers, glidants or lubricants. For example, the pharmaceutical compositions (e.g., a tablet) comprising the solid form of rifaximin further comprise microcrystalline cellulose as the filler, colloidal silicon dioxide as the glidant, and magnesium stearate as the lubricant. The amount of filler, glidant, and/or lubricant can vary depending upon the nature and amounts of other components. Typical amounts for fillers (e.g., microcrystalline cellulose) are e.g., from about 5 wt % to about 60 wt %, from about 10 wt % to about 55 wt %, from about 5 wt % to about 15 wt %, from about 8 wt % to about 13 wt %, from about 10 wt % to about 12 wt %, from about 10 wt % to about 19 wt %, about 11 wt %, from about 15 wt % to about 25 wt %, from about 17 wt % to about 19 wt %, about 18 wt %, from about 40 wt % to about 60 wt %, from about 45 wt % to about 55 wt %, from about 49 wt % to about 55 wt %, from about 49 wt % to about 51 wt %, from about 53 wt % to about 55 wt %, about 50 wt %, or about 54 wt % based on the total amount (wt %) of components in the pharmaceutical composition. Typical amounts for glidants (e.g., colloidal silicon dioxide) are e.g., from about 0.1 wt % to about 0.3 wt %, from about 0.15 wt % to about 0.25 wt %, or about 0.2 wt % based on the total amount (wt %) of components in the pharmaceutical composition. Typical amounts for lubricants (e.g., magnesium stearate) are e.g., from about 0.3 wt % to about 0.6 wt %, from about 0.4 wt % to about 0.6 wt %, from about 0.45 wt % to about 0.55 wt %, about 0.45 wt %, about 0.47 wt %, or about 0.49 wt % based on the total amount (wt %) of components in the pharmaceutical composition.

Thus, in one aspect, provided herein are pharmaceutical compositions (e.g., a tablet) comprising i) from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt % rifaximin and HMPC-AS in equal amounts based on the total amount (wt %) of components in the pharmaceutical composition; ii) from about 1 wt % to about 4 wt %, from about 3 wt % to about 5 wt %, from about 2 wt % to about 4 wt %, from about 1 wt % to about 2 wt %, about 1 wt %, about 2 wt %, about 3 wt %, or about 4 wt % poloxamer 407 based on the total amount (wt %) of components in the pharmaceutical composition; iii) from about 4 wt % to about 14 wt %, from about 12 wt % to about 14 wt %, about 13 wt %, from about 4 wt % to about 10 wt %, from about 4 wt % to about 6 wt %, about 5 wt %, from about 8% to about 10 wt %, or about 9 wt % croscarmellose sodium based on the total amount (wt %) of components in the pharmaceutical composition; and iv) from about 10 wt % to about 12 wt %, from about 10 wt % to about 19 wt %, from about 49 wt % to about 55 wt %, from about 53 wt % to about 55 wt %, from about 49 wt % to about 51 wt %, about 11 wt %, from about 17 wt % to about 19 wt %, about 18 wt %, from about 49 wt % to about 55 wt %, about 50 wt %, or about 54 wt % microcrystalline cellulose based on the total amount (wt %) of components in the pharmaceutical composition.

In another aspect, provided herein are pharmaceutical compositions (e.g., a tablet) comprising i) about 33 wt %, about 34 wt %, about 35 wt %, from about 16 wt % to about 18 wt %, or about 17 wt % rifaximin and HMPC-AS in equal amounts based on the total amount (wt %) of components in the pharmaceutical composition; ii) about 1 wt %, about 2 wt %, or about 4 wt % poloxamer 407 based on the total amount (wt %) of components in the pharmaceutical composition; iii) about 13 wt %, about 5 wt %, or about 9 wt % croscarmellose sodium based on the total amount (wt %) of components in the pharmaceutical composition; and iv) about 11 wt %, about 18 wt %, about 50 wt %, or about 54 wt % microcrystalline cellulose based on the total amount (wt %) of components in the pharmaceutical composition.

In another aspect, provided herein are pharmaceutical compositions (e.g., a tablet) comprising i) from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt % rifaximin and HMPC-AS in equal amounts based on the total amount (wt %) of components in the pharmaceutical composition; ii) from about 1 wt % to about 4 wt %, from about 3 wt % to about 5 wt %, from about 2 wt % to about 4 wt %, from about 1 wt % to about 2 wt %, about 1 wt %, about 2 wt %, about 3 wt %, or about 4 wt % poloxamer 407 based on the total amount (wt %) of components in the pharmaceutical composition; iii) from about 4 wt % to about 14 wt %, from about 12 wt % to about 14 wt %, about 13 wt %, from about 4 wt % to about 10 wt %, from about 4 wt % to about 6 wt %, about 5 wt %, from about 8% to about 10 wt %, or about 9 wt % croscarmellose sodium based on the total amount (wt %) of components in the pharmaceutical composition; iv) from about 10 wt % to about 12 wt %, from about 10 wt % to about 19 wt %, about 11 wt %, from about 17 wt % to about 19 wt %, from about 49 wt % to about 55 wt %, from about 53 wt % to about 55 wt %, from about 49 wt % to about 51 wt %, about 18 wt %, from about 49 wt % to about 55 wt %, about 50 wt %, or about 54 wt % microcrystalline cellulose based on the total amount (wt %) of components in the pharmaceutical composition; and v) from about 0.15 wt % to about 0.25 wt % or about 0.2 wt % colloidal silicon dioxide based on the total amount (wt %) of components in the pharmaceutical composition.

In another aspect, provided herein are pharmaceutical compositions (e.g., a tablet) comprising i) about 33 wt %, about 34 wt %, about 35 wt %, from about 16 wt % to about 18 wt %, or about 17 wt % rifaximin and HMPC-AS in equal amounts based on the total amount (wt %) of components in the pharmaceutical composition; ii) about 1 wt %, about 2 wt %, or about 4 wt % poloxamer 407 based on the total amount (wt %) of components in the pharmaceutical composition; iii) about 13 wt %, about 5 wt %, or about 9 wt % croscarmellose sodium based on the total amount (wt %) of components in the pharmaceutical composition; iv) about 11 wt %, about 18 wt %, about 50 wt %, or about 54 wt % microcrystalline cellulose based on the total amount (wt %) of components in the pharmaceutical composition; and v) about 0.2 wt % colloidal silicon dioxide based on the total amount (wt %) of components in the pharmaceutical composition.

In another aspect, provided herein are pharmaceutical compositions (e.g., a tablet) comprising i) from about 33 wt % to about 35 wt %, about 33 wt %, about 34 wt %, about 35 wt %, from about 16 wt % to about 34 wt %, from about 16 wt % to about 18 wt %, about 16 wt %, about 17 wt %, or about 18 wt % rifaximin and HMPC-AS in equal amounts based on the total amount (wt %) of components in the pharmaceutical composition; ii) from about 1 wt % to about 4 wt %, from about 3 wt % to about 5 wt %, from about 2 wt % to about 4 wt %, from about 1 wt % to about 2 wt %, about 1 wt %, about 2 wt %, about 3 wt %, or about 4 wt % poloxamer 407 based on the total amount (wt %) of components in the pharmaceutical composition; iii) from about 4 wt % to about 14 wt %, from about 12 wt % to about 14 wt %, about 13 wt %, from about 4 wt % to about 10 wt %, from about 4 wt % to about 6 wt %, about 5 wt %, from about 8% to about 10 wt %, or about 9 wt % croscarmellose sodium (crosslinked carboxymethyl cellulose sodium) based on the total amount (wt %) of components in the pharmaceutical composition; iv) from about 10 wt % to about 12 wt %, from about 49 wt % to about 55 wt %, from about 53 wt % to about 55 wt %, from about 49 wt % to about 51 wt %, from about 10 wt % to about 19 wt %, about 11 wt %, about 17 wt % to about 19 wt %, about 18 wt %, from about 49 wt % to about 55 wt %, about 50 wt %, or about 54 wt % microcrystalline cellulose based on the total amount (wt %) of components in the pharmaceutical composition; v) from about 0.15 wt % to about 0.25 wt % or about 0.2 wt % colloidal silicon dioxide based on the total amount (wt %) of components in the pharmaceutical composition; and vi) from about 0.45 wt % to about 0.55 wt %, about 0.45 wt %, about 0.47 wt %, or about 0.49 wt % magnesium stearate based on the total amount (wt %) of components in the pharmaceutical composition.

In another aspect, provided herein are pharmaceutical compositions (e.g., a tablet) comprising i) about 33 wt %, about 34 wt %, about 35 wt %, from about 16 wt % to about 18 wt %, or about 17 wt % rifaximin and HMPC-AS in equal amounts based on the total amount (wt %) of components in the pharmaceutical composition; ii) about 1 wt %, about 2 wt % or about 4 wt % poloxamer 407 based on the total amount (wt %) of components in the pharmaceutical composition; iii) about 13 wt %, about 5 wt %, or about 9 wt % croscarmellose sodium based on the total amount (wt %) of components in the pharmaceutical composition; iv) about 11 wt %, about 18 wt %, about 50 wt %, or about 54 wt % microcrystalline cellulose based on the total amount (wt %) of components in the pharmaceutical composition; v) about 0.2 wt % colloidal silicon dioxide based on the total amount (wt %) of components in the pharmaceutical composition; and vi) about 0.47 wt % magnesium stearate based on the total amount (wt %) of components in the pharmaceutical composition.

In specific embodiments, the pharmaceutical compositions comprise the ratios of components set forth in Table 37.

Other embodiments and aspects are disclosed infra.

### BRIEF DESCRIPTION OF THE DRAWINGS

FIG. 1. Chemical structure of Rifaximin.

FIG. 2. Overlay of XRPD patterns for Rifaximin/PVP K-90 dispersions obtained from methanol by spray drying.

FIG. 3. mDSC thermogram for 25:75 (w/w) Rifaximin/PVP K-90 dispersion obtained from methanol by spray drying.

FIG. 4. mDSC thermogram for 50:50 (w/w) Rifaximin/PVP K-90 dispersion obtained from methanol by spray drying.

FIG. 5. mDSC thermogram for 75:25 (w/w) Rifaximin/PVP K-90 dispersion obtained from methanol by spray drying.

FIG. 6. Overlay of mDSC thermogram for Rifaximin/PVP K-90 dispersions obtained from methanol by spray drying.

FIG. 7. Overlay of XRPD patterns for Rifaximin/HPMC-P dispersions obtained from methanol by spray drying.

FIG. 8. mDSC thermogram for 25:75 (w/w) Rifaximin/HPMC-P dispersion obtained from methanol by spray drying.

FIG. 9. mDSC thermogram for 50:50 (w/w) Rifaximin/HPMC-P dispersion obtained from methanol by spray drying.

FIG. 10. mDSC thermogram for 75:25 (w/w) Rifaximin/HPMC-P dispersion obtained from methanol by spray drying.

FIG. 11. Overlay of mDSC thermogram for Rifaximin/HPMC-P dispersions obtained from methanol by spray drying.

FIG. 12. Overlay of XRPD patterns for Rifaximin/HPMC-AS HG dispersions obtained from methanol by spray drying.

FIG. 13. mDSC thermogram for 25:75 (w/w) Rifaximin/HPMC-AS HG dispersion obtained from methanol by spray drying.

FIG. 14. mDSC thermogram for 50:50 (w/w) Rifaximin/HPMC-AS HG dispersion obtained from methanol by spray drying.

FIG. 15. mDSC thermogram for 75:25 (w/w) Rifaximin/HPMC-AS HG dispersion obtained from methanol by spray drying.

FIG. 16. Overlay of mDSC thermogram for Rifaximin/HPMC-AS HG dispersions obtained from methanol by spray drying.

FIG. 17. Overlay of XRPD patterns for Rifaximin/HPMC-AS MG dispersions obtained from methanol by spray drying.

FIG. 18. mDSC thermogram for 25:75 (w/w) Rifaximin/HPMC-AS MG dispersion obtained from methanol by spray drying.

FIG. 19. mDSC thermogram for 50:50 (w/w) Rifaximin/HPMC-AS MG dispersion obtained from methanol by spray drying.

FIG. 20. mDSC thermogram for 75:25 (w/w) Rifaximin/HPMC-AS MG dispersion obtained from methanol by spray drying.

FIG. 21. Overlay of mDSC thermogram for Rifaximin/HPMC-AS MG dispersions obtained from methanol by spray drying.

FIG. 22. Overlay of XRPD patterns for Rifaximin/Eudragit L100-55 dispersions obtained from methanol by spray drying.

FIG. 23. mDSC thermogram for 25:75 (w/w) Rifaximin/Eudragit L100-55 dispersion obtained from methanol by spray drying.

FIG. 24. mDSC thermogram for 50:50 (w/w) Rifaximin/Eudragit L100-55 dispersion obtained from methanol by spray drying.

FIG. 25. mDSC thermogram for 75:25 (w/w) Rifaximin/Eudragit L100-55 dispersion obtained from methanol by spray drying.

FIG. 26. Overlay of mDSC thermogram for Rifaximin/Eudragit L100-55 dispersions obtained from methanol by spray drying.

FIG. 27. mDSC thermogram for 25:75 (w/w) Rifaximin/HPMC-P dispersion stressed at 40° C./75% RH for 7 d.

FIG. 28. mDSC thermogram for 75:25 (w/w) Rifaximin/HPMC-AS HG dispersion stressed at 40° C./75% RH for 7 d.

FIG. 29. mDSC thermogram for 75:25 (w/w) Rifaximin/HPMC-AS MG dispersion stressed at 40° C./75% RH for 7 d.

FIG. 30. mDSC thermogram for 25:75 (w/w) Rifaximin/Eudragit L100-55 dispersion stressed at 40° C./75% RH for 7 d.

FIG. 31. XRPD pattern for 50:50 (w/w) Rifaximin/HPMC-AS MG dispersion.

FIG. 32. Modulate DSC thermograms for 50:50 (w/w) Rifaximin/HPMC-AS MG dispersion.

FIG. 33. TG-IR analysis for 50:50 (w/w) Rifaximin/HPMC-AS MG dispersion—TGA data.

FIG. 34. TG-IR analysis for 50:50 (w/w) Rifaximin/HPMC-AS MG dispersion—Gram-Schmidt plot and waterfall plot.

FIG. 35. TG-IR analysis for 50:50 (w/w) Rifaximin/HPMC-AS MG dispersion.

FIG. 36. XRPD pattern for 25:75 (w/w) Rifaximin/HPMC-P dispersion.

FIG. 37. Modulate DSC thermograms for 25:75 (w/w) Rifaximin/HPMC-P dispersion.

FIG. 38. TG-IR analysis for 25:75 (w/w) Rifaximin/HPMC-P dispersion—TGA data.

FIG. 39. TG-IR analysis for 25:75 (w/w) Rifaximin/HPMC-P dispersion—Gram-Schmidt plot and waterfall plot.

FIG. 40. TG-IR analysis for 25:75 (w/w) Rifaximin/HPMC-P dispersion.

FIG. 41. Overlay of pre-processed XRPD patterns in multivariate mixture analysis.

FIG. 42. Estimated Concentrations of Rifaximin (blue) and HPMC-AS MG (red) using Unscrambler MCR analysis.

FIG. 43. Estimated XRPD patterns of Rifaximin (blue) and HPMC-AS MG (red) using Unscrambler MCR analysis.

FIG. 44. Overlay of estimated XRPD pattern of pure rifaximin using MCR and measured XRPD pattern of 100% rifaximin.

FIG. 45. Overlay of estimated XRPD pattern of pure HPMC-AS MG using MCR and measured XRPD pattern of 100% HPMC-AS MG.

FIG. 46. An exemplary XRPD pattern for combined solids of Rifaximin/HPMC-AS MG/Pluronic ternary dispersion.

FIG. 47. A modulate DSC thermogram for combined solids of Rifaximin/HPMC-AS MG/Pluronic ternary dispersion.

FIG. 48. A TG-IR analysis for combined solids of Rifaximin/HPMC-AS MG/Pluronic ternary dispersion—TGA thermogram.

FIG. 49. An exemplary TG-IR analysis for combined solids of Rifaximin/HPMC-AS MG/Pluronic ternary dispersion.

FIG. 50. An exemplary overlay of IR spectra for X-ray amorphous Rifaximin and combined solids of Rifaximin/HPMC-AS MG/Pluronic ternary dispersion.

FIG. 51. An exemplary overlay of Ramam spectra for X-ray amorphous Rifaximin and combined solids of Rifaximin/HPMC-AS MG/Pluronic ternary dispersion.

FIG. 52. A particle size analysis report for combined solids of Rifaximin/HPMC-AS MG/Pluronic ternary dispersion.

FIG. 53. An exemplary dynamic vapor sorption (DVS) analysis for combined solids of Rifaximin/HPMC-AS MG/Pluronic ternary dispersion.

FIG. 54. An exemplary overlay of XRPD patterns for Rifaximin/HPMC-AS MG/Pluronic ternary dispersion post-DVS solids and solids as-prepared.

FIG. 55. An exemplary overlay of XRPD patterns for Rifaximin ternary dispersion post-stressed samples and as-prepared sample.

FIG. 56. An exemplary mDSC thermogram for Rifaximin ternary dispersion after 70° C./75% RH 1 week.

FIG. 57. An exemplary mDSC thermogram for Rifaximin ternary dispersion after 70° C./75% RH 3 weeks.

FIG. 58. An exemplary mDSC thermogram for Rifaximin ternary dispersion after 40° C./75% RH 6 weeks.

FIG. 59. An exemplary mDSC thermogram for Rifaximin ternary dispersion after 40° C./75% RH 12 weeks.

FIG. 60. Pharmacokinetic data of solid dispersion in dogs.

FIG. 61. Rifaximin SD capsules dissolution; acid phase: 0.1 N HCl with variable exposure time. Buffer phase: pH 6.8 with 0.45% SDS.

FIG. 62. Rifaximin SD capsules dissolution; acid phase: 2 hours; buffer phase: pH 6.8.

FIG. 63. Rifaximin capsule dissolution; phosphate buffer pH 6.8 with 0.45% SDS.

Rifaximin spray dried dispersion (SDD) capsule dissolution. FIG. 64A acid phase 2 hours, buffer phase: P. Buffer, pH. 7.4. FIG. 64B acid phase: 0.1N HCl with various exposure times, buffer phase: P. buffer, pH 7.4 with 0.45% SDS. FIG. 64C shows the general structure of hydroxypropyl methylcellulose (HMPC). FIG. 64D represents the percent released at 30 min as a function of pH.

Rifamixin SDD with 10% CS formulation. FIG. 65A kinetic solubility Rifamixin SD granules. 10% wt % CS sodium FaSSIF, 10% wt % CS sodium FeSSIF. FIG. 65B dissolution profiles SDD tablet 10% CS. 0.2% SLS, pH4.5; 0.2% SLS, pH5.5; 0.2% SLS, pH 7.4; FaSSIF.

Rifaximin SDD with 10% CS formulation. Rifaxamin SDD capsules dissolution: FIG. 66A acid phase 2 hours, buffer phase: P. Buffer, pH. 7.4. With 0.45% SDS; without SDS. FIG. 66B acid phase: 0.1N HCl with variable exposure times, buffer phase: P. buffer, pH 7.4 with 0.45% SDS.

Effects of media pH on dissolution. FIG. 67A Rifaxamin SDD tablet dissolution. Acid phase: 2 hours, pH 2.0, FIG. 67B Dissolution profiles 0.2% SDS at pH 4.5, SDD tablet dissolution at various levels of CS: 0%, 2.5%, 5%, and 10% CS.

Effects of media pH on dissolution. FIG. 68A Rifaxamin SDD tablet dissolution at various levels of CS: 0%, 2.5%, 5%, and 10% CS, 0.2% SDS at pH 5.5. FIG. 68B Dissolution profiles SDD tablet dissolution at various levels of CS: 0%, 2.5%, 5%, and 10% CS, 0.2% SDS at pH 7.4.

Effects of media pH on dissolution. FIG. 69A Rifaxamin SDD tablet dissolution 2.5% CS, 0.2% SLS, pH4.5, 0.2% SLS, pH 5.5, 0.2% SLS, pH 7.4. FIG. 69B Rifaxamin SDD tablet dissolution 0% CS, 0.2% SLS, pH4.5, 0.2% SLS, pH 5.5, 0.2% SLS, pH 7.4.

Effects of media pH on dissolution. FIG. 70A Rifaxamin SDD tablet dissolution 10% CS, 0.2% SLS, pH4.5, 0.2% SLS, pH 5.5, 0.2% SLS, pH 7.4. FIG. 70B Rifaxamin SDD tablet dissolution 5% CS, 0.2% SLS, pH4.5, 0.2% SLS, pH 5.5, 0.2% SLS, pH 7.4.

CS release mechanism. FIG. 71A Kinetic solubility in FaSSIF media, pH 6.5, FIG. 71B slope vs. time point.

FIG. 72 depicts an overlay of XRPD patterns of rifaximin quaternary samples spray dried from methanol. The top is a rifaximin quaternary sample containing 0.063 wt % BHA. The second is rifaximin quaternary sample containing 0.063 wt % BHT. The third: is rifaximin quaternary sample containing 0.094 wt % PG, and the bottom is a spray dried rifaximin ternary dispersion.

FIG. 73 depicts an mDSC thermogram of rifaximin quaternary sample containing 0.063 wt % BHA.

FIG. 74 depicts an mDSC thermogram of rifaximin quaternary sample containing 0.063 wt % BHT.

FIG. 75 depicts a mDSC thermogram of rifaximin quaternary sample containing 0.094 wt % PG.

FIG. 76 depicts an XRPD pattern comparison of rifaximin solid dispersion powder 42.48% w/w with roller compacted material of rifaximin blend. Top: Rifaximin Solid Dispersion Powder 42.48% w/w; Bottom: roller compacted rifaximin blend.

FIG. 77 depicts the pharmacokinetics of rifaximin following administration of varying forms and formulations following a single oral dose of 2200 mg in dogs.

FIG. 78 depicts Rifaximin SDD in dogs.

FIG. 79 depicts the quotient study design.

FIG. 80 summarizes the dose escalation/regional absorption study, part A dose escalation/dose selection.

FIG. 81 depicts representative subject data from a dose escalation study.

FIG. 82 depicts representative subject data from a dose escalation study.

FIG. 83 depicts mean dose escalation data, on a linear scale.

FIG. 84 depicts mean dose escalation data, on a log scale.

FIG. 85 depicts a summary of Rifaximin SDD dose escalation studies.

FIG. 86 is a Table of dose/dosage form comparison.

FIG. 87 is a Table of dose/dosage form comparison. This table compares SDD at increasing doses to the current crystalline formulation in terms of systemic PK.

FIG. 88 presents a Kaplan-Meier estimate for the distribution of time to hospitalization for any of the liver cirrhosis complications by treatment group for an ITT (intent-to-treat) population with a formulation comprising rifaximin solid dispersion.

FIG. 89 presents a Kaplan-Meier estimate for the distribution of time to all-cause mortality by treatment group for an ITT population with a formulation comprising rifaximin solid dispersion.

FIG. 90 presents a Kaplan-Meier estimate for the distribution of time to hospitalization for any of the liver cirrhosis complications or all-cause mortality by treatment group for the ITT population with a formulation comprising rifaximin solid dispersion.

FIG. 91 presents a Kaplan-Meier estimate for the distribution of time to hospitalization for any of the liver cirrhosis complications or all-cause mortality by treatment group for the PP (per protocol) population with a formulation comprising rifaximin solid dispersion.

### DETAILED DESCRIPTION

Embodiments described herein relate to the discovery of new solid dispersion forms of rifaximin with a variety of polymers and polymer concentrations. In one embodiment the use of one or more of new solid dispersion forms of the antibiotic known as Rifaximin (INN (international non-proprietary name)), in the manufacture of medicinal preparations for the oral or topical route is contemplated. For example, the solid dispersion forms of rifaximin are used to create pharmaceutical compositions, e.g., tablets or capsules, or microgranules comprising solid dispersion forms of rifaximin Exemplary methods for producing rifaximin microgranules are set forth in the examples. Rifaximin microgranules can be formulated into pharmaceutical compositions as described herein.

Embodiments described herein also relate to administration of such medicinal preparations to a subject in need of treatment with antibiotics. Provided herein are solid dispersion forms of rifaximin with a variety of polymers and polymer concentrations.

As used herein, the term “intragranular release controlling agent” includes agents that cause a pharmaceutical composition, e.g., a microgranule, to breakdown thereby releasing the active ingredient, e.g., rifaximin. Exemplary intragranular release controlling agent, include disintegrants such as crosprovidone, sodium starch glycolate, corn starch, microcrystalline cellulose, cellulosic derivatives (such as croscarmellose sodium (crosslinked carboxymethyl cellulose sodium)), sodium bicarbonate, and sodium alginate.

In one embodiment, the intragranular release controlling agent comprises between about 2 wt % to about 40 wt % of the microgranule, about 5 wt % to about 20 wt % of the microgranule, about 8-15 wt % or about 10 wt % of the microgranule.

In another embodiment, the intragranular release controlling agent comprises between about 2 wt % to about 14 wt % of the microgranule.

In another embodiment, the microgranule comprises a surfactant, e.g., a non-ionic surfactant. In one embodiment, the non-ionic surfactant comprises between about 2 wt % to about 10 wt % of the microgranule, between about 4 wt % to about 8 wt % of the microgranule, about 6 to about 7 wt % of the microgranule, or about 5.0 wt % of the microgranule.

In another embodiment, the microgranule comprises an antioxidant. In one embodiment, the antioxidant comprises between about 0.1 wt % to about 3 wt % of the microgranule, between 0.3 wt % to about 2 wt % or between about 0.5 wt % to about 1 wt % of the microgranule.

As used herein, the term “intragranular” refers to the components that reside within the microgranule. As used herein, the term “extragranular” refers to the components of the pharmaceutical composition that are not contained within the microgranule.

As used herein, the term polymorph is occasionally used as a general term in reference to the forms of rifaximin and includes within the context, salt, hydrate, polymorph co-crystal and amorphous forms of rifaximin. This use depends on context and will be clear to one of skill in the art.

As used herein, the term “about” when used in reference to x-ray powder diffraction pattern peak positions refers to the inherent variability of the peaks depending on, for example, the calibration of the equipment used, the process used to produce the polymorph, the age of the crystallized material and the like, depending on the instrumentation used. In this case the measure variability of the instrument was about +0.2 degrees 2-θ. A person skilled in the art, having the benefit of this disclosure, would understand the use of “about” in this context. The term “about” in reference to other defined parameters, e.g., water content, Cmax, tmax, AUC, intrinsic dissolution rates, temperature, and time, indicates the inherent variability in, for example, measuring the parameter or achieving the parameter. A person skilled in the art, having the benefit of this disclosure, would understand the variability of a parameter as connoted by the use of the word about.

As used herein, “similar” in reference to a form exhibiting characteristics similar to, for example, an XRPD, an IR, a Raman spectrum, a DSC, TGA, NMR, SSNMR, etc, indicates that the polymorph or cocrystal is identifiable by that method and could range from similar to substantially similar, so long as the material is identified by the method with variations expected by one of skill in the art according to the experimental variations, including, for example, instruments used, time of day, humidity, season, pressure, room temperature, etc.

As used herein, “rifaximin solid dispersion,” “rifaximin ternary dispersion,” “solid dispersion of rifaximin,” “solid dispersion”, “solid dispersion forms of rifaximin”, “SD”, “SDD”, and “form solid dispersion of rifaximin” are intended to have equivalent meanings and include rifaximin polymer dispersion composition. These compositions are XRPD amorphous, but distinguishable from XRPD of amorphous rifaximin. As shown in the Examples and Figures, the rifaximin polymer dispersion compositions are physically chemically distinguishable from amorphous rifaximin, including different Tg, different XRPD profiles and different dissolution profiles.

Polymorphism, as used herein, refers to the occurrence of different crystalline forms of a single compound in distinct hydrate status, e.g., a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as solubility profiles, melting point temperatures, hygroscopicity, particle shape, density, flowability, compactibility and/or x-ray diffraction peaks. The solubility of each polymorph may vary, thus, identifying the existence of pharmaceutical polymorphs is essential for providing pharmaceuticals with predictable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. For a general review of polymorphs and the pharmaceutical applications of polymorphs see G. M. Wall, Pharm Manuf. 3, 33 (1986); J. K. Haleblian and W. McCrone, J Pharm. Sci., 58, 911 (1969); and J. K. Haleblian, J. Pharm. Sci., 64, 1269 (1975), all of which are incorporated herein by reference.

As used herein, “subject” includes organisms which are capable of suffering from a bowel disorder or other disorder treatable by rifaximin or who could otherwise benefit from the administration of rifaximin solid dispersion compositions as described herein, such as human and non-human animals. The term “non-human animals” includes all vertebrates, e.g., mammals, e.g., rodents, e.g., mice, and non-mammals, such as non-human primates, e.g., sheep, dog, cow, chickens, amphibians, reptiles, etc. Susceptible to a bowel disorder is meant to include subjects at risk of developing a bowel disorder infection, e.g., subjects suffering from one or more of an immune suppression, subjects that have been exposed to other subjects with a bacterial infection, physicians, nurses, subjects traveling to remote areas known to harbor bacteria that causes travelers' diarrhea, subjects who drink amounts of alcohol that damage the liver, subjects with a history of hepatic dysfunction, etc.

The language “a prophylactically effective amount” of a composition refers to an amount of a rifaximin solid dispersion formulation or otherwise described herein which is effective, upon single or multiple dose administration to the subject, in preventing or treating a bacterial infection.

The language “therapeutically effective amount” of a composition refers to an amount of a rifaximin solid dispersion effective, upon single or multiple dose administration to the subject to provide a therapeutic benefit to the subject. In one embodiment, the therapeutic benefit is wounding or killing a bacterium, or in prolonging the survivability of a subject with such a bowel or skin disorder. In another embodiment, the therapeutic benefit is inhibiting a bacterial infection or prolonging the survival of a subject with such a bacterial infection beyond that expected in the absence of such treatment.

Rifaximin exerts a broad antibacterial activity in the gastrointestinal tract against localized gastrointestinal bacteria that cause infectious diarrhea, including anaerobic strains. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

In respect to possible adverse events coupled to the therapeutic use of rifaximin, the induction of bacterial resistance to the antibiotics is of particular relevance.

From this point of view, any differences found in the systemic absorption of the forms of rifaximin disclosed herein may be significant, because at sub-inhibitory concentration of rifaximin, such as in the range from 0.1 to 1 μg/ml, selection of resistant mutants has been demonstrated to be possible (Marchese A. et al. In vitro activity of rifaximin, metronidazole and vancomycin against clostridium difficile and the rate of selection of spontaneously resistant mutants against representative anaerobic and aerobic bacteria, including ammonia-producing species. Chemotherapy, 46(4), 253-266, (2000)).

Forms, formulations and compositions of rifaximin have been found to have differing in vivo bioavailability properties. Thus, the polymorphs disclosed herein would be useful in the preparation of pharmaceuticals with different characteristics for the treatment of infections. This would allow generation of rifaximin preparations that have significantly different levels of adsorption with Cₘₐₓ, values from about 0.0 ng/ml to 5.0 μg/ml. This leads to preparation of rifaximin compositions that are from negligibly to significantly adsorbed by subjects undergoing treatment. One embodiment described herein is modulating the therapeutic action of rifaximin by selecting the proper form, formulation and/or composition, or mixture thereof, for treatment of a subject. For example, in the case of invasive bacteria, the most bioavailable form, formulation and/or composition can be selected from those disclosed herein, whereas in case of non-invasive pathogens less adsorbed forms, formulations and/or compositions of rifaximin can be selected, since they may be safer for the subject undergoing treatment. A form, formulation and/or composition of rifaximin may determine solubility, which may also determine bioavailability.

For XRPD analysis, accuracy and precision associated with third party measurements on independently prepared samples on different instruments may lead to variability which is greater than ±0.1° 2θ. For d-space listings, the wavelength used to calculate d-spacings was 1.541874 Å, a weighted average of the Cu-Kα1 and Cu-Kα2 wavelengths. Variability associated with d-spacing estimates was calculated from the USP recommendation, at each d-spacing, and provided in the respective data tables and peak lists.

### Methods of Treatment

Provided herein are methods of treating, preventing, or alleviating bowel related disorders comprising administering to a subject in need thereof an effective amount of one or more of the solid dispersion compositions of rifaximin. Bowel related disorders include one or more of irritable bowel syndrome, diarrhea, microbe associated diarrhea, Clostridium difficile associated diarrhea, travelers' diarrhea, small intestinal bacterial overgrowth, Crohn's disease, diverticular disease, chronic pancreatitis, pancreatic insufficiency, enteritis, colitis, hepatic encephalopathy, minimal hepatic encephalopathy or pouchitis.

The length of treatment for a particular bowel disorder will depend in part on the disorder. For example, travelers' diarrhea may only require treatment duration of 12 to about 72 hours, while Crohn's disease may require treatment durations from about 2 days to 3 months. Dosages of rifaximin will also vary depending on the diseases state. Proper dosage ranges are provided herein infra. The polymorphs and cocrystals described herein may also be used to treat or prevent apathology in a subject suspected of being exposed to a biological warfare agent.

The identification of those subjects who are in need of prophylactic treatment for bowel disorder is well within the ability and knowledge of one skilled in the art. Certain of the methods for identification of subjects which are at risk of developing a bowel disorder which can be treated by the subject method are appreciated in the medical arts, such as family history, travel history and expected travel plans, the presence of risk factors associated with the development of that disease state in the subject. A clinician skilled in the art can readily identify such candidate subjects, by the use of, for example, clinical tests, physical examination and medical/family/travel history.

Topical skin infections and vaginal infections may also be treated with the rifaximin compositions described herein. Thus, described herein are methods of using a solid dispersion composition of rifaximin (SD rifaximin compositions) to treat vaginal infections, ear infections, lung infections, periodontal conditions, rosacea, and other infections of the skin and/or other related conditions. Provided herein are vaginal pharmaceutical compositions to treat vaginal infection, particularly bacterial vaginosis, to be administered topically, including vaginal foams and creams, containing a therapeutically effective amount of SD rifaximin compositions, preferably between about 50 mg and 2500 mg. Pharmaceutical compositions known to those of skill in the art for the treatment of vaginal pathological conditions by the topical route may be advantageously used with SD rifaximin compositions. For example, vaginal foams, ointments, creams, gels, ovules, capsules, tablets and effervescent tablets may be effectively used as pharmaceutical compositions containing SD rifaximin compositions, which may be administered topically for the treatment of vaginal infections, including bacterial vaginosis. Also provided herein are method of using SD rifaximin compositions to treat gastric dyspepsia, including gastritis, gastroduodenitis, antral gastritis, antral erosions, erosive duodenitis and peptic ulcers. These conditions may be caused by the Helicobacter pylori. Pharmaceutical formulations known by those of skill in the art with the benefit of this disclosure to be used for oral administration of a drug may be used. Provided herein are methods of treating ear infections with SD rifaximin compositions. Ear infections include external ear infection, or a middle and inner ear infection. Also provided herein are methods of using SD rifaximin compositions to treat or prevent aspiration pneumonia and/or sepsis, including the prevention of aspiration pneumonia and/or sepsis in patients undergoing acid suppression or undergoing artificial enteral feedings via a Gastrostomy/Jejunostomy or naso/oro gastric tubes; prevention of aspiration pneumonia in patients with impairment of mental status, for example, for any reason, for subjects undergoing anesthesia or mechanical ventilation that are at high risk for aspiration pneumonia. Provided herein are methods to treat or to prevent periodontal conditions, including plaque, tooth decay and gingivitis. Provided herein are methods of treating rosacea, which is a chronic skin condition involving inflammation of the cheeks, nose, chin, forehead, or eyelids.

Also provided herein are methods of using the solid dispersions of rifaximin and pharmaceutical compositions thereof to prevent complications of liver cirrhosis such as e.g., in subjects with early decompensation.

Also provided herein are methods of using the solid dispersions of rifaximin and pharmaceutical compositions thereof to prevent all-cause mortality e.g., in subjects with liver cirrhosis who may also have early decompensation.

Also provided herein are methods of using the solid dispersions of rifaximin and pharmaceutical compositions thereof to reduce the time to hospitalization that is associated with complications of liver disease (e.g., liver cirrhosis complications) such as e.g., reducing the time to hospitalization from one or more of hepatic encephalopathy (HE), esophageal variceal bleeding (EVB), spontaneous bacterial peritonitis (SBP), and hepatorenal syndrome (HRS).

Also provided herein are methods of using the solid dispersions of rifaximin and pharmaceutical compositions thereof to prevent hospitalization that is associated with complications of liver disease (e.g., liver cirrhosis complications) such as e.g., reducing the time to hospitalization from one or more of hepatic encephalopathy (HE), esophageal variceal bleeding (EVB), spontaneous bacterial peritonitis (SBP), and hepatorenal syndrome (HRS).

Also provided herein are methods of using the solid dispersions of rifaximin and pharmaceutical compositions thereof to reduce the time to all-cause mortality that is associated with complications of liver disease (e.g., liver cirrhosis complications) such as e.g., reducing the time to all-cause mortality from one or more of hepatic encephalopathy (HE), esophageal variceal bleeding (EVB), spontaneous bacterial peritonitis (SBP), and hepatorenal syndrome (HRS).

Also provided herein are methods of using the solid dispersions of rifaximin and pharmaceutical compositions thereof to prevent all-cause mortality that is associated with complications of liver disease (e.g., liver cirrhosis complications) such as e.g., reducing the time to all-cause mortality from one or more of hepatic encephalopathy (HE), esophageal variceal bleeding (EVB), spontaneous bacterial peritonitis (SBP), and hepatorenal syndrome (HRS).

Further provided are methods of using the solid dispersions of rifaximin and pharmaceutical compositions thereof to reduce the time to development of refractory ascites in e.g., subjects having early decompensated liver cirrhosis or liver cirrhosis complications such as HE, EVB, SBP, or HRS.

As used for the purposes of preventing complications of liver cirrhosis, preventing all-cause mortality, reducing or preventing the time to hospitalization that is associated with complications of liver disease, and reducing the time to or preventing all-cause mortality that is associated with complications of liver disease, HE is defined as an altered mental status diagnosed as HE and defined as an increase of the Conn score to Grade ≧2 (ie, 0 or 1 to ≧2).

As used for the purposes of preventing complications of liver cirrhosis, preventing all-cause mortality, reducing or preventing the time to hospitalization that is associated with complications of liver disease, and reducing the time to or preventing all-cause mortality that is associated with complications of liver disease, EVB is defined as the occurrence of a clinically significant gastrointestinal bleed being defined as 1) bleeding from an esophageal or gastric varix at the time of endoscopy or 2) the presence of large varices with blood evident in the stomach, and no other identifiable cause of bleeding observed during endoscopy, and at least one or more of the following criteria is present: i) drop in hemoglobin of greater than 2 g/dL over the first 48 hours post hospital admission, ii) transfusion requirement of 2 units of blood or more within 24 hours of hospital admission, iii) a systolic blood pressure of less than 100 mm Hg, or iv) pulse rate greater than 100 beat/min at the time of admission.

As used for the purposes of preventing complications of liver cirrhosis, preventing all-cause mortality, reducing or preventing the time to hospitalization that is associated with complications of liver disease, and reducing the time to or preventing all-cause mortality that is associated with complications of liver disease, SBP is defined as greater than 250 polymorphonuclear (PMN) cells/mm³ and/or positive monomicrobial culture in the ascitic fluid.

As used for the purposes of preventing complications of liver cirrhosis, preventing all-cause mortality, reducing or preventing the time to hospitalization that is associated with complications of liver disease, and reducing the time to or preventing all-cause mortality that is associated with complications of liver disease, HRS is defined as i) progressive rise in serum creatinine (>1.5 mg/dL) with no improvement after at least 2 days with diuretic withdrawal and volume expansion with albumin, ii) absence of parenchymal kidney disease, iii) oliguria, iv) absence of shock, and v) no current or recent (within 3 months prior randomization) treatment with nephrotoxic drugs.

Time to development of medically refractory ascites is defined as ascites which can either no longer be effectively managed by i) a low sodium diet and maximal doses of diuretics (e.g., up to 400 mg spironolactone and 160 mg furosemide per day) or ii) diuretics, due to the inability to tolerate side effects of maximal doses of diuretics.

### Pharmaceutical Preparations

Embodiments also provide pharmaceutical compositions, comprising an effective amount of one or more SD rifaximin compositions, or microgranules comprising SD forms of rifaximin described herein (e.g., described herein and a pharmaceutically acceptable carrier). In a further embodiment, the effective amount is effective to treat a bacterial infection, e.g., small intestinal bacterial overgrowth, Crohn's disease, hepatic encephalopathy, antibiotic associated colitis, and/or diverticular disease. Embodiments also provide pharmaceutical compositions, comprising an effective amount of rifaximin SD compositions.

For examples of the use of rifaximin to treat Travelers' diarrhea, see Infante R M, Ericsson C D, Zhi-Dong J, Ke S, Steffen R, Riopel L, Sack D A, DuPont, HL, Enteroaggregative Escherichia coli Diarrhea in Travelers: Response to Rifaximin Therapy. Clinical Gastroenterology and Hepatology. 2004; 2:135-138; and Steffen R, M.D., Sack DA, M.D., Riopel L, Ph.D., Zhi-Dong J, Ph.D., Sturchler M, M.D., Ericsson CD, M.D., Lowe B, M. Phil., Waiyaki P, Ph.D., White M, Ph.D., DuPont H L, M.D. Therapy of Travelers' Diarrhea With Rifaximin on Various Continents. The American Journal of Gastroenterology. May 2003, Volume 98, Number 5, all of which are incorporated herein by reference in their entirety. Examples of treating hepatic encephalopathy with rifaximin see, for example, N. Engl J Med. 2010_362_1071-1081.

Embodiments also provide pharmaceutical compositions comprising rifaximin SD compositions and a pharmaceutically acceptable carrier. Embodiments of the pharmaceutical composition further comprise excipients, for example, one or more of a diluting agent, binding agent, lubricating agent, intragranular release controlling agent, e.g., a disintegrating agent, coloring agent, flavoring agent or sweetening agent. One composition may be formulated for selected coated and uncoated tablets, hard and soft gelatin capsules, sugar-coated pills, lozenges, wafer sheets, pellets and powders in sealed packet. For example, compositions may be formulated for topical use, for example, ointments, pomades, creams, gels and lotions.

In one embodiment, tablet compositions may be formulated as immediate release (IR) tablets or sustained extended release (SER) tablets. IR tablets as used herein are designed to release the entire drug content (e.g., rifaximin solid dispersion) upon contact with the dissolution medium. SER tablets as used herein are designed to release the drug (e.g., rifaximin solid dispersion) slowly over a period of 3 to 4 hours after contact with the dissolution medium.

In an embodiment, the rifaximin SD composition is administered to the subject using a pharmaceutically-acceptable formulation, e.g., a pharmaceutically-acceptable formulation that provides sustained or delayed delivery of the SD rifaximin composition to a subject for at least 2, 4, 6, 8, 10, 12 hours, 24 hours, 36 hours, 48 hours, one week, two weeks, three weeks, or four weeks after the pharmaceutically-acceptable formulation is administered to the subject. The pharmaceutically-acceptable formulations may contain microgranules comprising rifaximin as described herein.

In certain embodiments, these pharmaceutical compositions are suitable for topical or oral administration to a subject. In other embodiments, as described in detail below, the pharmaceutical compositions described herein may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those SD rifaximin compositions and cocrystals presented herein, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is preferably “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Methods of preparing these compositions include the step of bringing into association a SD rifaximin composition(s) or microgranules containing the SD rifaximin compositions with the carrier and, optionally, one or more accessory ingredients. In general, the formulations are prepared by uniformly and intimately bringing into association a SD rifaximin composition with liquid carriers, or finely divided solid carriers, or both, and then, if necessary, shaping the product.

Compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a SD rifaximin composition(s) as an active ingredient. A compound may also be administered as a bolus, electuary or paste.

The SD compositions of rifaximin disclosed herein can be advantageously used in the production of medicinal preparations having antibiotic activity, containing rifaximin, for both oral and topical use. The medicinal preparations for oral use will contain an SD composition of rifaximin together with the usual excipients, for example diluting agents such as mannitol, lactose and sorbitol; binding agents such as starches, gelatines, sugars, cellulose derivatives, natural gums and polyvinylpyrrolidone; lubricating agents such as talc, stearates, hydrogenated vegetable oils, polyethylenglycol and colloidal silicon dioxide; disintegrating agents such as starches, celluloses, alginates, gums and reticulated polymers; coloring, flavoring, disintegrants, and sweetening agents.

Embodiments described herein include SD rifaximin composition administrable by the oral route, for instance coated and uncoated tablets, of soft and hard gelatin capsules, sugar-coated pills, lozenges, wafer sheets, pellets and powders in sealed packets or other containers.

Pharmaceutical compositions for rectal or vaginal administration may be presented as a suppository, which may be prepared by mixing one or more SD rifaximin composition(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent. Compositions which are suitable for vaginal administration also include pessaries, tampons, creams, gels, pastes, foams or spray formulations containing such carriers as are known in the art to be appropriate.

Dosage forms for the topical or transdermal administration of a SD rifaximin composition(s) include powders, sprays, ointments, pastes, creams, lotions, gels, solutions, patches and inhalants. The active SD rifaximin composition(s) may be mixed under sterile conditions with a pharmaceutically-acceptable carrier, and with any preservatives, buffers, or propellants which may be required.

Ointments, pastes, creams and gels may contain, in addition to SD rifaximin composition(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a SD rifaximin composition(s), excipients such as lactose, talc, silicic acid, aluminium hydroxide, calcium silicates and polyamide powder, or mixtures of these substances. Sprays can additionally contain customary propellants, such as chlorofluorohydrocarbons and volatile unsubstituted hydrocarbons, such as butane and propane.

The SD rifaximin composition(s) can be alternatively administered by aerosol. This is accomplished by preparing an aqueous aerosol, liposomal preparation or solid particles containing the compound. A non-aqueous (e.g., fluorocarbon propellant) suspension could be used. Sonic nebulizers are preferred because they minimize exposing the agent to shear, which can result in degradation of the compound.

An aqueous aerosol is made, for example, by formulating an aqueous solution or suspension of the agent together with conventional pharmaceutically-acceptable carriers and stabilizers. The carriers and stabilizers vary with the requirements of the particular compound, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a SD rifaximin composition(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions suitable for parenteral administration may comprise one or more SD rifaximin composition(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

When the SD rifaximin composition(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the SD rifaximin composition(s) are formulated into pharmaceutically-acceptable dosage forms by methods known to those of skill in the art.

Actual dosage levels and time course of administration of the active ingredients in the pharmaceutical compositions may be varied so as to obtain an amount of the active ingredient which is effective to achieve the desired therapeutic response for a particular subject, composition, and mode of administration, without being toxic to the subject. An exemplary dose range is from 25 to 3000 mg per day. Other doses include, for example, 600 mg/day, 1100 mg/day and 1650 mg/day. Other exemplary doses include, for example, 1000 mg/day, 1500 mg/day, from between 500 mg to about 1800 mg/day or any value in-between.

In one aspect, the solid dispersion form of rifaximin is formulated as a tablet comprising from about 30 to about 100 mg, or from about 40 to about 80 mg, rifaximin per tablet. In another aspect, the solid dispersion form of rifaximin is formulated as a tablet comprising about 40 mg rifaximin or about 80 mg rifaximin.

A preferred dose of the SD rifaximin composition disclosed herein is the maximum that a subject can tolerate without developing serious side effects. Preferably, the SD rifaximin composition is administered at a rifaximin concentration from about 0.5 mg to about 200 mg, or from about 1 mg to about 200 mg, or from about 0.7 mg to about 100 mg, or from about 0.7 mg to about 50 mg, or from about 0.7 mg to about 10 mg, or from about 1 mg to about 5 mg, per kilogram of body weight. Ranges intermediate to the above-recited values are also intended to be part. For example, doses may range from 20 mg to about 2000 mg/day, or from 50 mg to about 2000 mg/day, or from 50 mg to about 1000 mg/day, or from 50 mg to about 500 mg/day.

In combination therapy treatment, the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the other therapeutic agent's optimal effective-amount range. In one embodiment in which another therapeutic agent is administered to an animal, the effective amount of the rifaximin SD composition is less than its effective amount in case the other therapeutic agent is not administered. In another embodiment, the effective amount of the conventional agent is less than its effective amount in case the rifaximin SD composition is not administered. In this way, undesired side effects associated with high doses of either agent may be minimized. Other potential advantages (including without limitation improved dosing regimens and/or reduced drug cost) will be apparent to those skilled in the art.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same subject's visit.

In certain embodiments, one or more compounds and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same compounds may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than a SD rifaximin composition may be repeated and the administration may be separated by at least 1 day, 2 days, 3 days, 5 days, 10 days, 15 days, 30 days, 45 days, 2 months, 75 days, 3 months, or at least 6 months.

Certain indications may require longer treatment times. For example, travelers' diarrhea treatment may only last from between about 12 hours to about 72 hours, while a treatment for Crohn's disease may be from between about 1 day to about 3 months. A treatment for hepatic encephalopathy may be, for example, for the remainder of the subject's life span. A treatment for IBS may be intermittent for weeks or months at a time or for the remainder of the subject's life.

### Compositions and Formulations

Rifaximin solid dispersions, pharmaceutical compositions comprising SD rifaximin or microgranules comprising rifaximin solid dispersions, can be made from, for example, polymers including polyvinylpyrrolidone (PVP) grade K-90, hydroxypropyl methylcellulose phthalate (HPMC-P) grade 55, hydroxypropyl methylcellulose acetate succinate (HPMC-AS) grades HG and MG, and a polymethacrylate (Eudragit® L100-55). Rifaximin solid dispersion compositions are comprised of, for example, 10:90, 15:85, 20:80, 25:75, 30:70, 40:60, 50:50 60:40, 70:30, 75:25, 80:20, 85:15, and 90:10 (Rifaximin/polymer, by weight). Preferred solid dispersions are comprised of 25:75, 50:50 and 75:25 (Rifaximin/polymer, by weight). In addition to rifaximin and polymer, solid dispersions may also comprise surfactants, for example, non-ionic, surfactant polyols. In addition to rifaximin and polymer, solid dispersions may also comprise dissolution enhancers (e.g., croscarmellose sodium) and surfactants, for example, non-ionic, surfactant polyols such as poloxamer 407.

An example of a formulation comprises about 50:50 (w/w) Rifaximin:HPMC-AS MG with from between about 2 wt % to about 10 wt % of a non-ionic, surfactant polyol, for example, Pluronic F-127.

One example of a formulation comprises 50:50 (w/w) Rifaximin:HPMC-AS MG with about 5.9 wt %) of a non-ionic, surfactant polyol, for example, Pluronic F-127. Spray dried rifaximin ternary dispersion (50:50 (w/w) rifaximin:HPMC-AS MG with 5.9 wt % Pluronic F-127) was blended with 10 wt % croscarmellose sodium and then filled into gelatin capsules. Each capsule contains 275 mg of rifaximin and the blend formulation is 85:5:10 of 50:50 (w/w) Rifaximin:HPMC-AS MG:Pluronic:croscarmellose sodium (calculated in total solids). Other examples of microgranules and pharmaceutical compositions comprising SD rifaximin are described in the examples.

One example of a solid dispersion of rifaximin comprises from about 46 wt % to about 48 wt % rifaximin, from about 46 wt % to about 48 wt % HPMC-AS, and from about 4 wt % to about 6 wt % poloxamer 407. Other examples of solid dispersion of rifaximin and pharmaceutical compositions comprising SD rifaximin are described in the examples.

Another example of a solid dispersion of rifaximin comprises about 47.2 wt % rifaximin, about 47.2 wt % HPMC-AS, and about 5.6 wt % poloxamer 407. Other examples of solid dispersion of rifaximin and pharmaceutical compositions comprising SD rifaximin are described in the examples.

To form the rifaximin solid dispersion, the components, e.g., rifaximin, polymer and methanol are mixed and then spray dried. Exemplary conditions are summarized in Table 9 and the procedure outlined below and in Examples 3 and 4.

Exemplary Spray Drying Process Parameters, include for example:
Spray Dryer—e.g., PSD 1;
Single or multi-fluid nozzle: e.g., a two Fluid Niro Nozzle;
Nozzle orifice—0.1-10 mm;
Inlet gas temperature—75-150±5 deg C.;
Process gas flow (mmH2O)—20-70, preferred 44;
Atomizing gas pressure—0.7-1 bar;
Feed rate—2-7 kg/Hr;
Outlet temperature—30-70±3 deg C.;
Solution temperature—20-50 deg C.; and
Post spray drying vacuum dry at 20-60 deg C., for between about 2 and 72 hrs.

### Article of Manufacture

Another embodiment includes articles of manufacture that comprise, for example, a container holding a rifaximin SD pharmaceutical composition suitable for oral or topical administration of rifaximin in combination with printed labeling instructions providing a discussion of when a particular dosage form should be administered with food and when it should be taken on an empty stomach. Exemplary dosage forms and administration protocols are described infra. The composition will be contained in any suitable container capable of holding and dispensing the dosage form and which will not significantly interact with the composition and will further be in physical relation with the appropriate labeling. The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

Another aspect is an article of manufacture that comprises a container containing a pharmaceutical composition comprising SD rifaximin composition or formulation wherein the container holds preferably rifaximin composition in unit dosage form and is associated with printed labeling instructions advising of the differing absorption when the pharmaceutical composition is taken with and without food.

Packaged compositions are also provided, and may comprise a therapeutically effective amount of rifaximin. Rifaximin SD composition and a pharmaceutically acceptable carrier or diluent, wherein the composition is formulated for treating a subject suffering from or susceptible to a bowel disorder, and packaged with instructions to treat a subject suffering from or susceptible to a bowel disorder.

Kits are also provided herein, for example, kits for treating a bowel disorder in a subject. The kits may contain, for example, one or more of the solid dispersion forms of rifaximin and instructions for use. The instructions for use may contain proscribing information, dosage information, storage information, and the like.

Packaged compositions are also provided, and may comprise a therapeutically effective amount of an SD rifaximin composition and a pharmaceutically acceptable carrier or diluent, wherein the composition is formulated for treating a subject suffering from or susceptible to a bowel disorder, and packaged with instructions to treat a subject suffering from or susceptible to a bowel disorder.

The present invention is further illustrated by the following examples, which should not be construed as further limiting. The contents of all figures and all references, patents and published patent applications cited throughout this application, as well as the Figures, are expressly incorporated herein by reference in their entirety.

### EXAMPLES

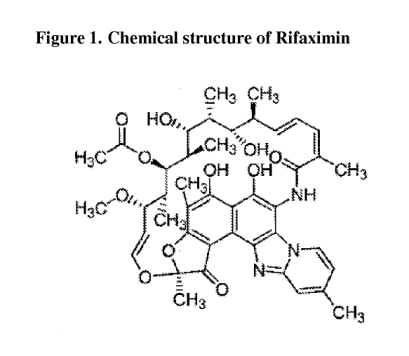

The chemical structure of Rifaximin is shown below in FIG. 1.

### Example 1. Solid Dispersions of Rifaximin

Various polymers were formulated with rifaximin into solids prepared by methanol and spray drying at small scale (˜1 g). Polymers, including polyvinylpyrrolidone (PVP) grade K-90, hydroxypropyl methylcellulose phthalate (HPMC-P) grade 55, hydroxypropyl methylcellulose acetate succinate (HPMC-AS) grades HG and MG, and a polymethacrylate (Eudragit® L100-55) were used. Solids have compositions of 25:75, 50:50 and 75:25 (Rifaximin/polymer, by weight).

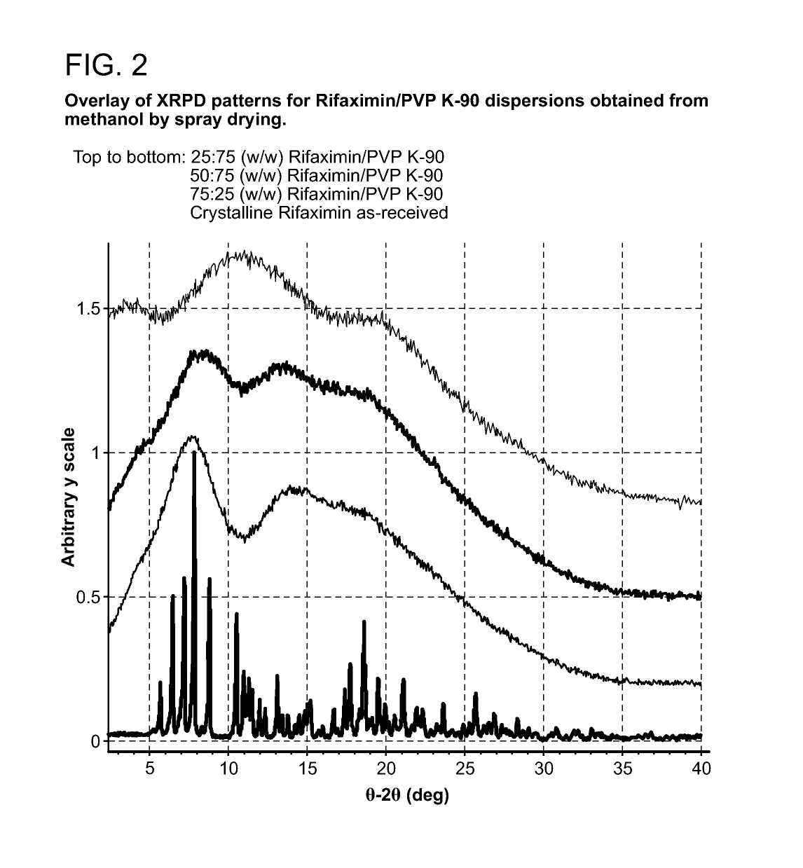

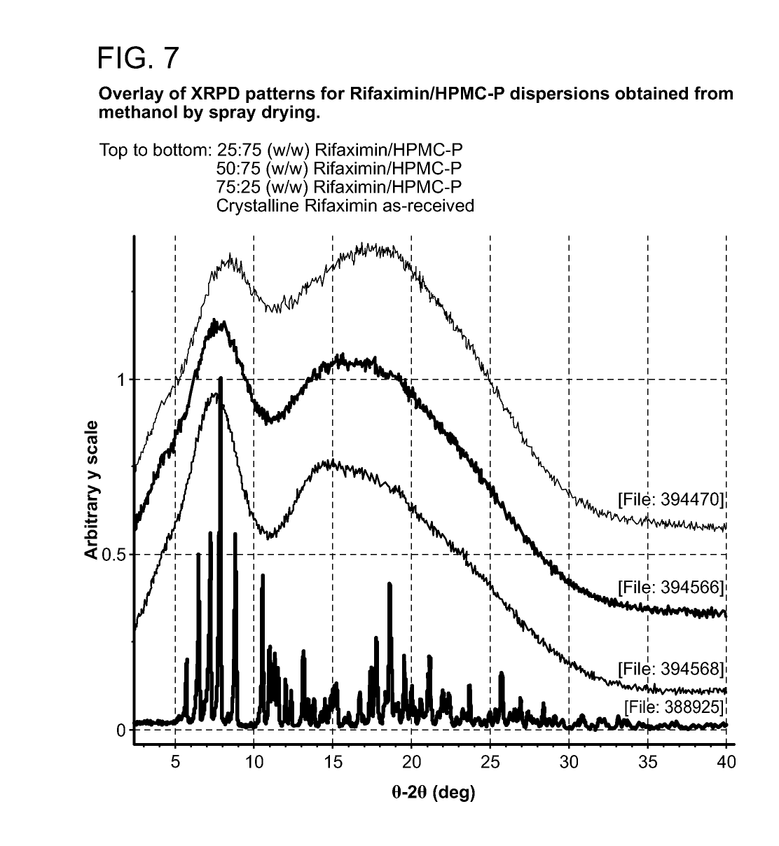

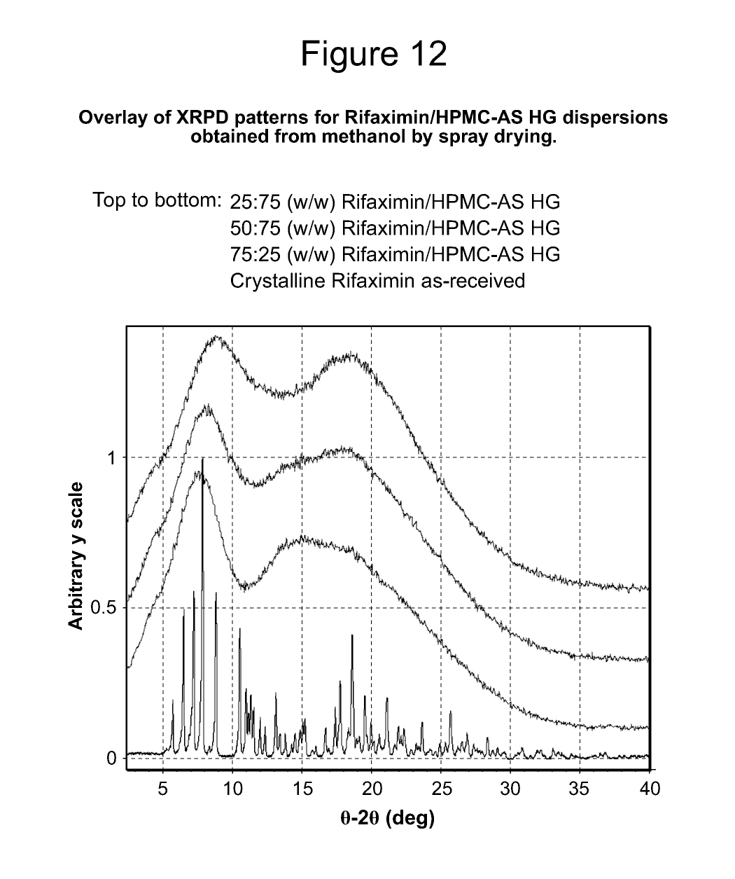

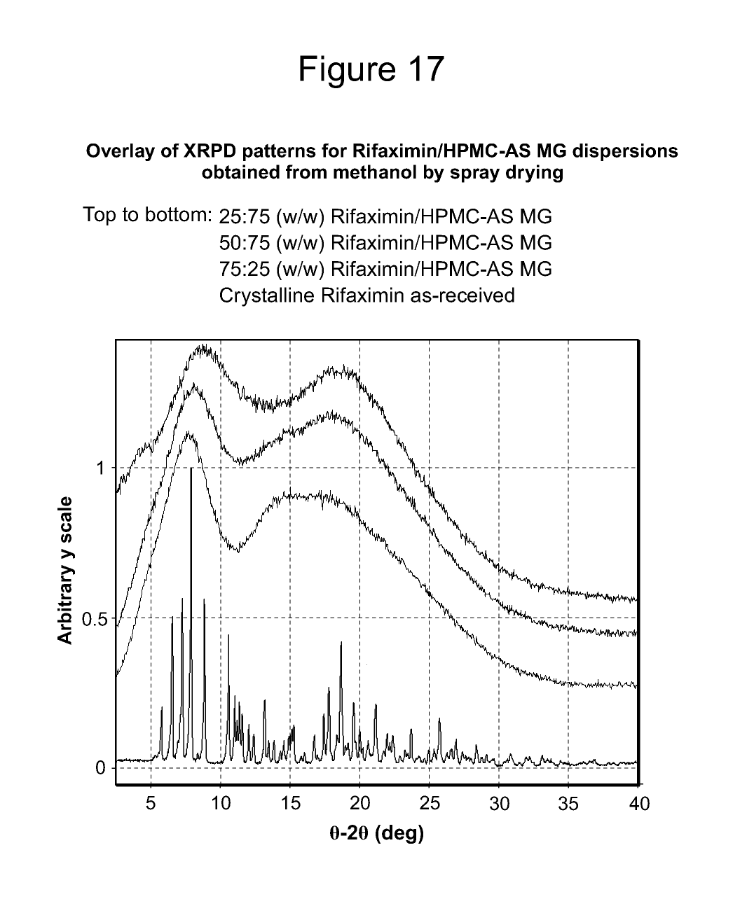

Samples generated were observed under polarized light microscope after preparation and were characterized by XRPD. The results are included in Table 1 through Table 5. Birefringence with extinction (B/E) was not observed for any of the samples, indicating solids without crystalline order were obtained. No sharp peaks were evident by visual inspection of XRPD patterns of these samples, consistent with non-crystalline materials, as shown in FIG. 2 (with PVP K-90), FIG. 7 (with HPMC-P), FIG. 12 (with HMPC-AS HG), FIG. 12 (with HMPC-AS MG), and FIG. 17 (with Eudragit L100-55).

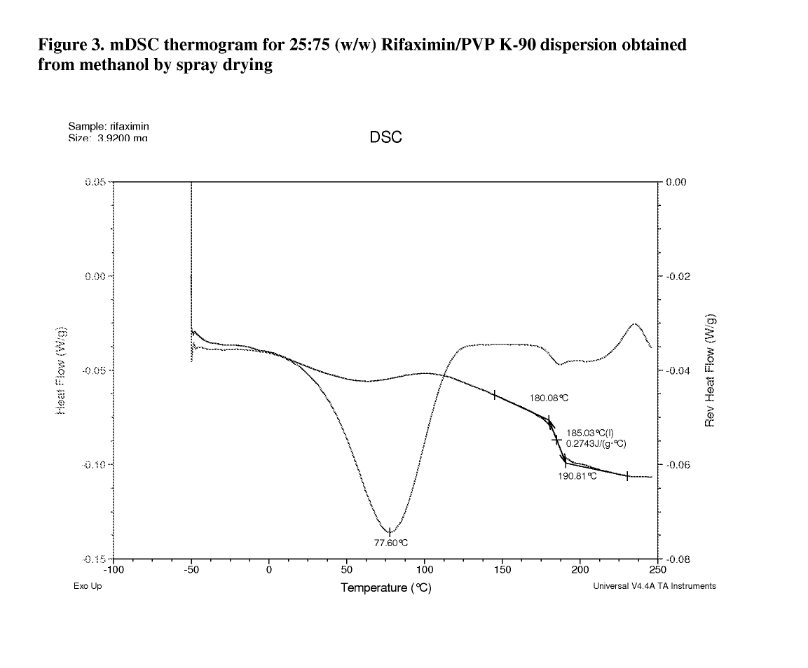

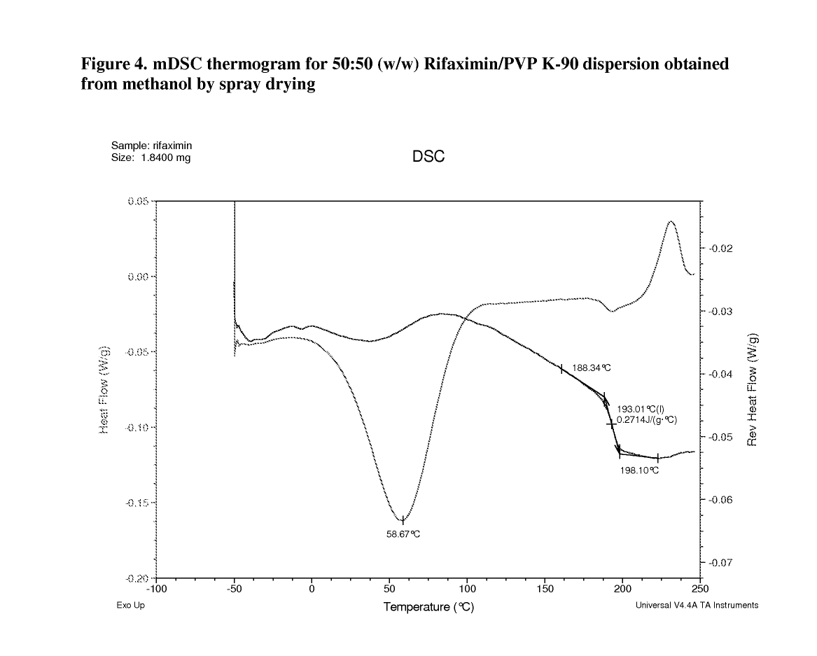

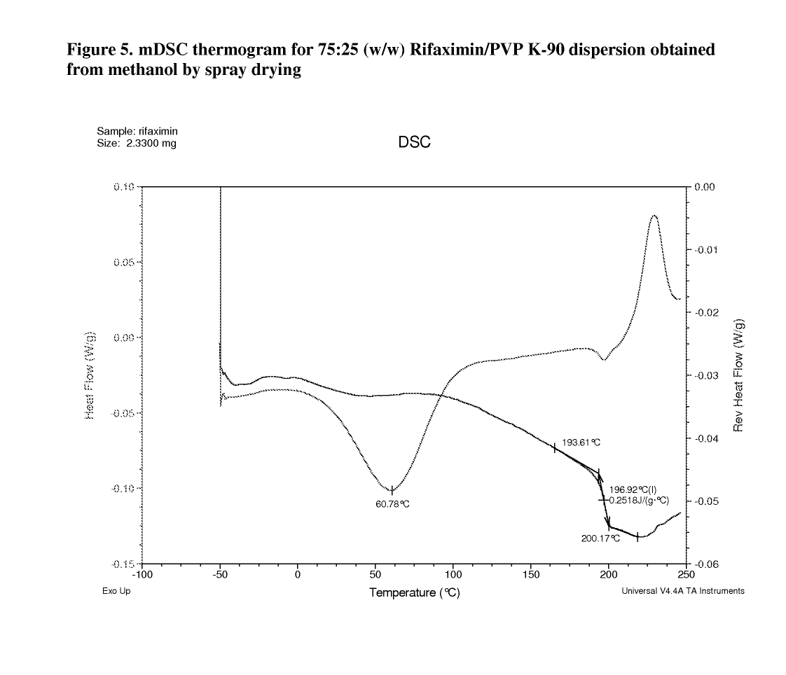

Materials were characterized by mDSC where the appearance of a single glass transition temperature (Tg), provides support for a non-crystalline fully miscible dispersion. All the dispersions prepared with PVP K-90 display a single apparent Tg at approximately 185° C. (FIG. 3, 25:75 w/w), 193° C. (FIG. 4, 50:50 w/w), and 197° C. (FIG. 5, 75:25) respectively. The change in heat capacity (ΔCp) at Tg is approximately 0.3 J/g·° C. for each dispersion. A non-reversible endotherm, which is likely due to the residual solvent in the materials, was observed in each of Rifaximin/PVP K-90 dispersions centered at approximately 78° C., 59° C. and 61° C.

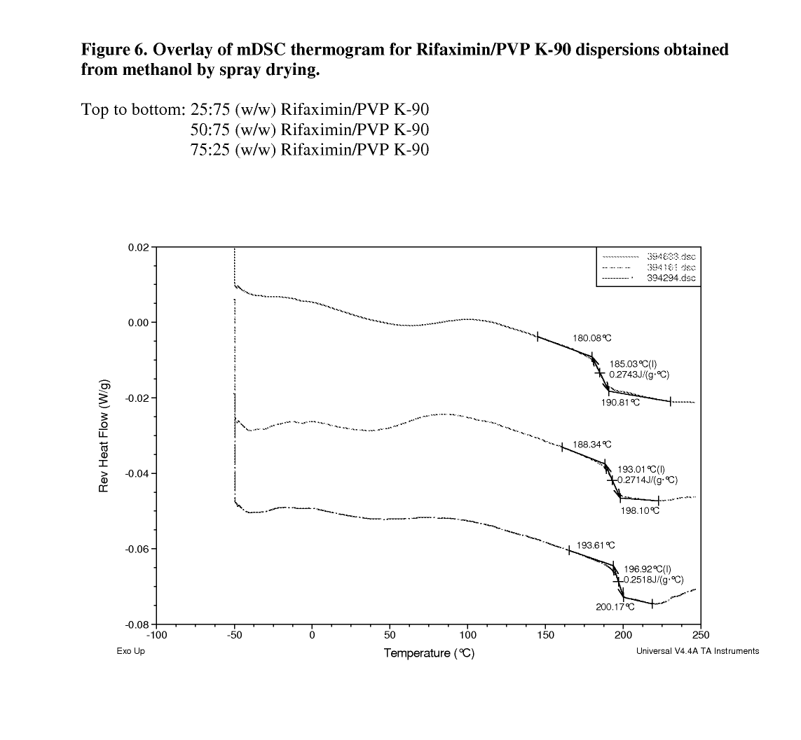

From FIG. 6, Tg of Rifaximin/PVP K-90 dispersions increases with the increased Rifaximin concentration, which is due to the higher Tg of Rifaximin (199° C.) than PVP K-90 (174° C.). Evidence of a single Tg may suggest that the components of the dispersion are intimately mixed, or miscible.

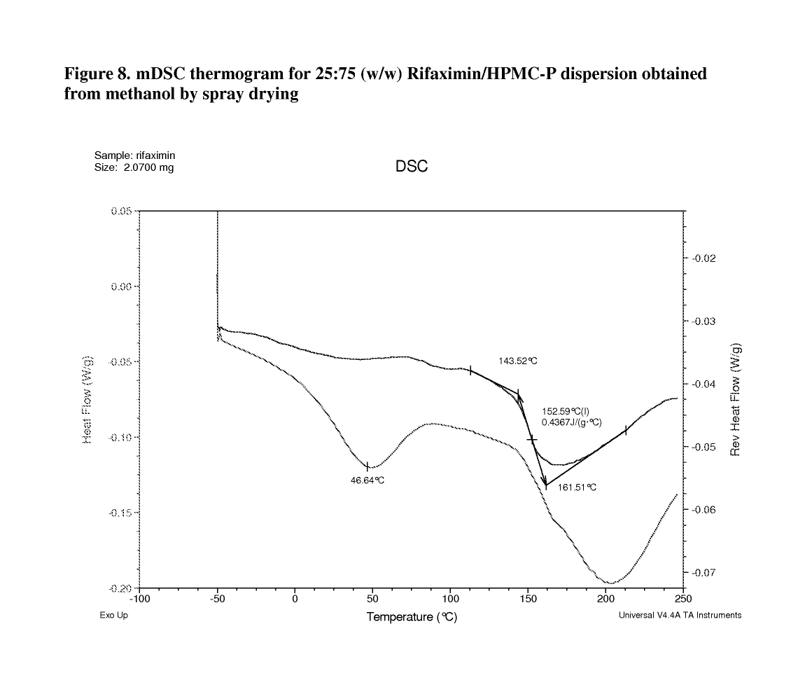

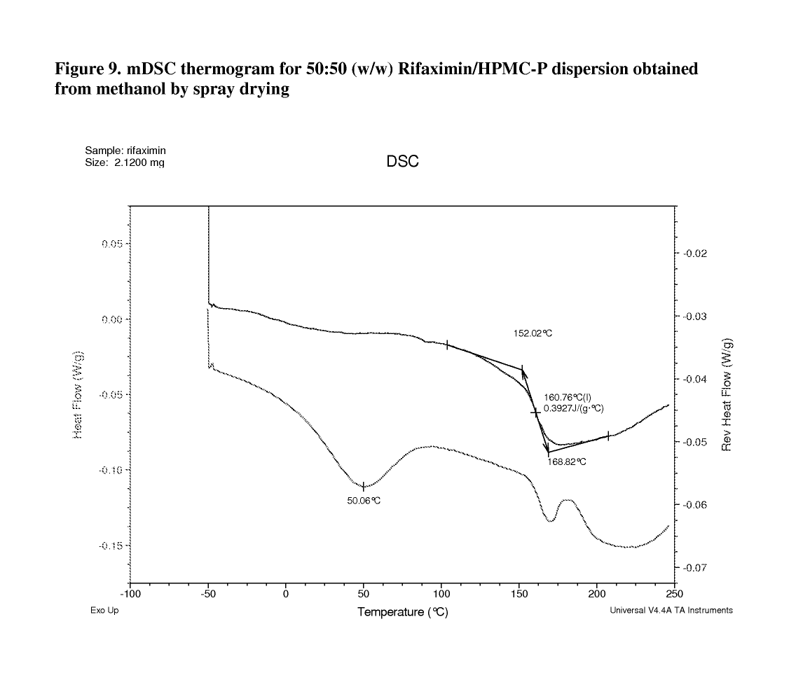

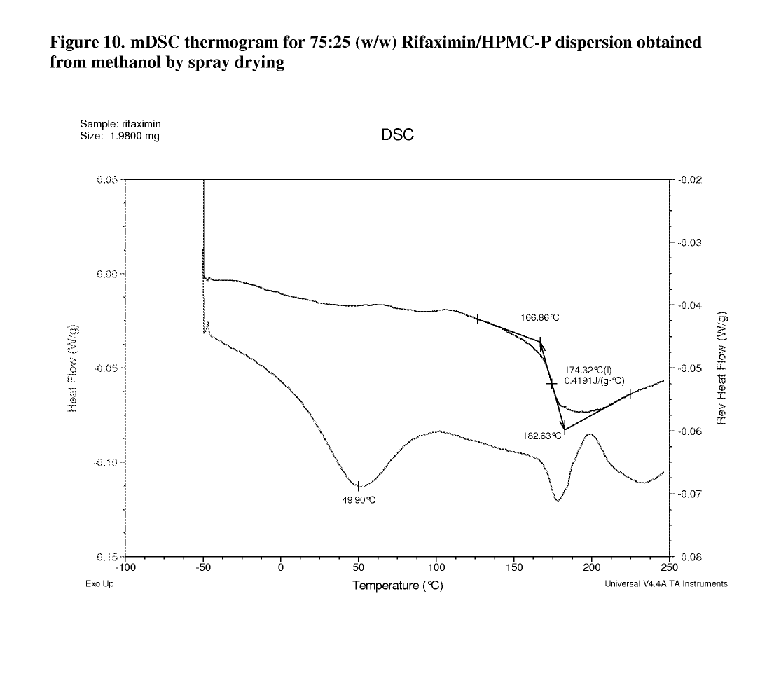

Dispersions prepared with other polymers also display a single apparent Tg, as a step change in the reversing heat flow signal by mDSC. Dispersions prepared with HPMC-P exhibit Tg at 153° C. (FIG. 8, 25:75 w/w), 161° C. (FIG. 9, 50:50 w/w) and 174° C. (FIG. 10, 75:25 w/w) respectively, with ΔCp at Tg approximately 0.4 J/g·° C.

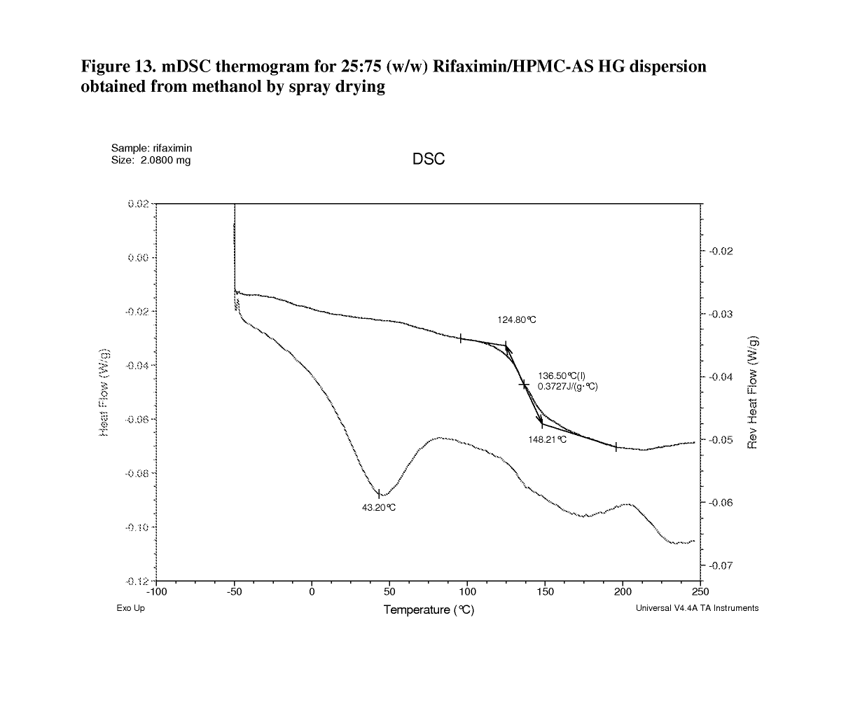

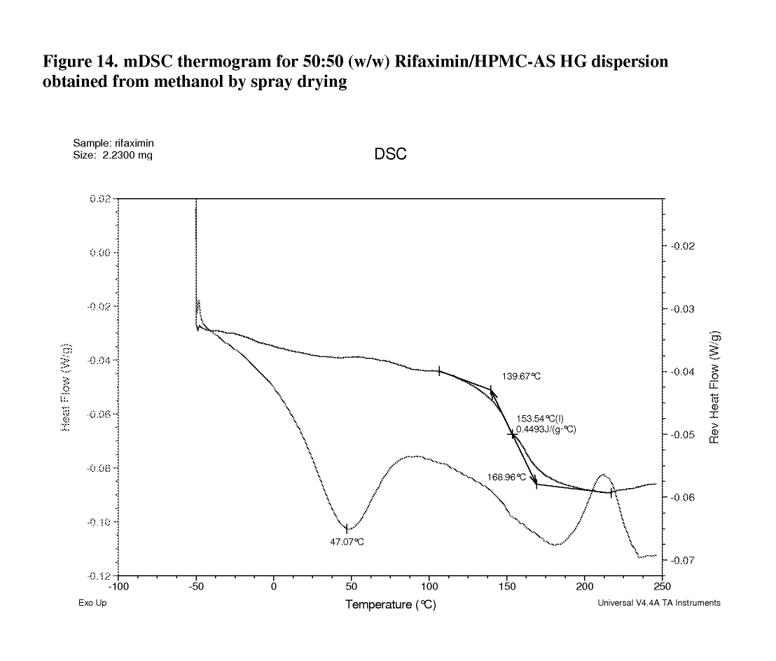

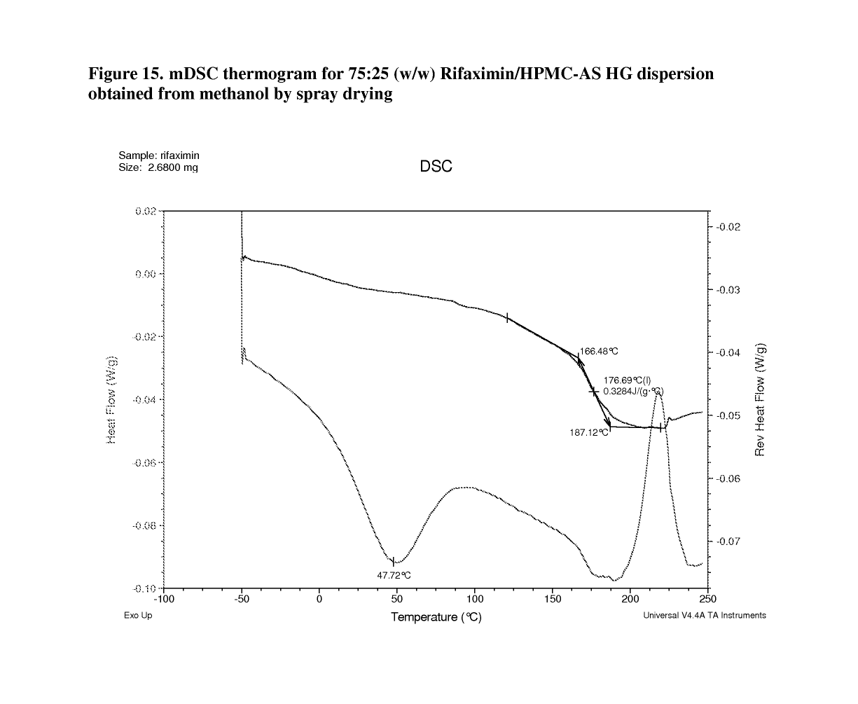

With HPMC-AS HG, dispersions display Tg at 137° C. (FIG. 13, 25:75 w/w), 154° C. (FIG. 14, 50:50 w/w) and 177° C. (FIG. 15, 75:25 w/w) respectively; ΔCp at Tg is approximately 0.4 or 0.3 J/g·° C.

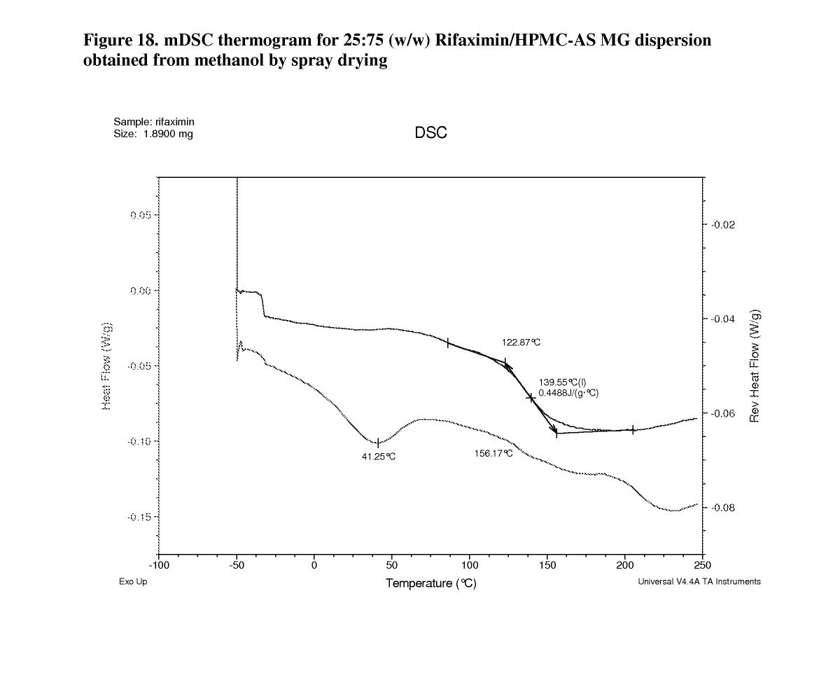

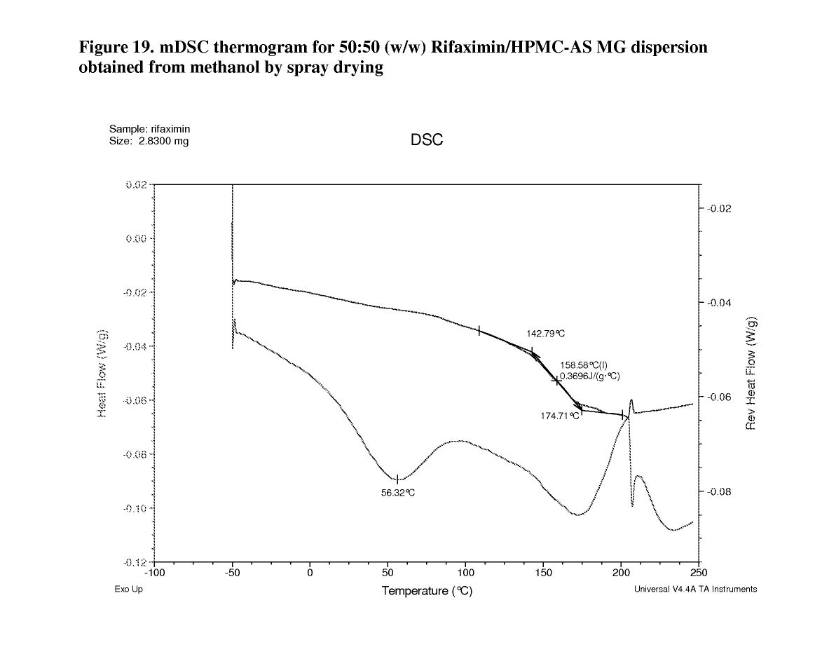

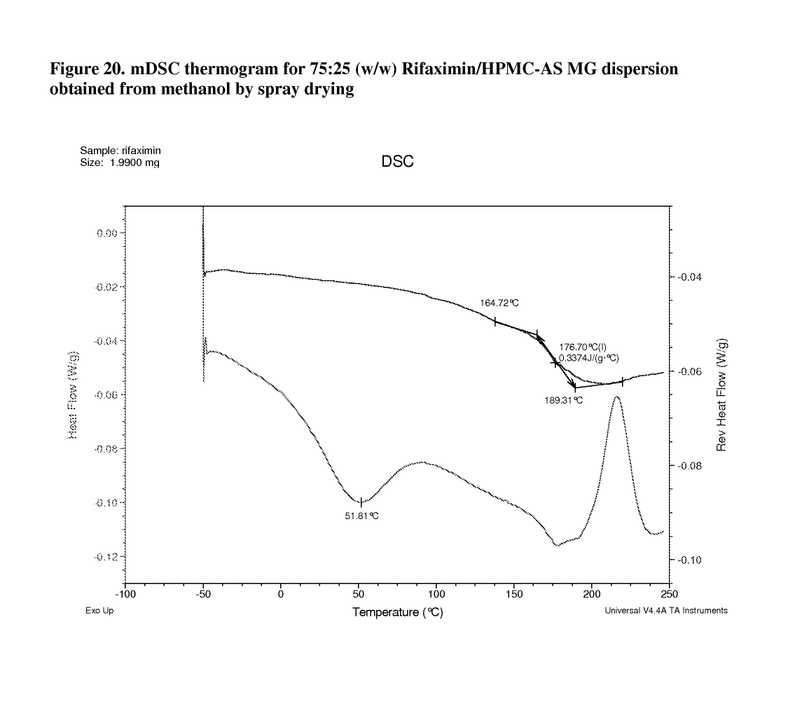

With HPMC-AS MG, dispersions display Tg at 140° C. (FIG. 18, 25:75 w/w), 159° C. (FIG. 19, 50:50 w/w) and 177° C. (FIG. 10, 75:25 w/w) respectively; ΔCp at Tg is approximately 0.4 or 0.3 J/g·° C.

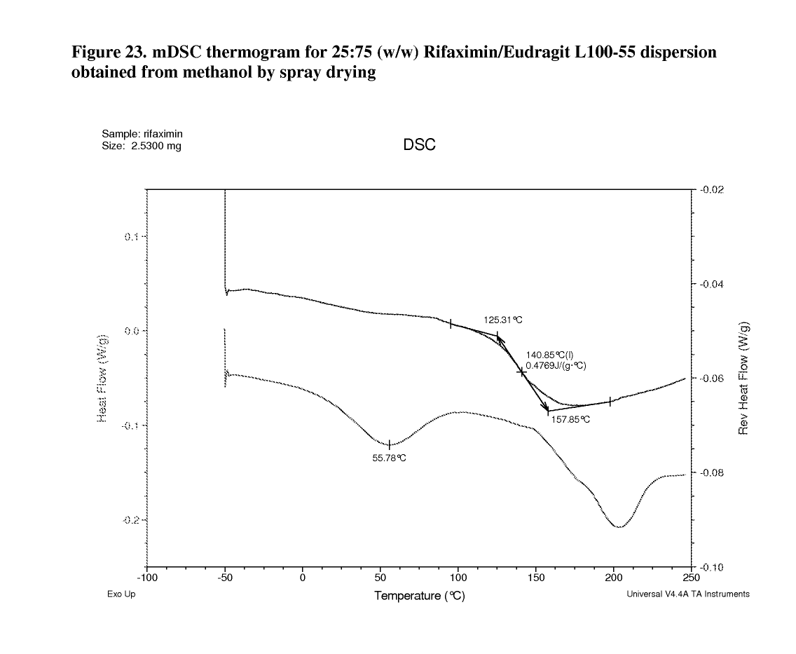

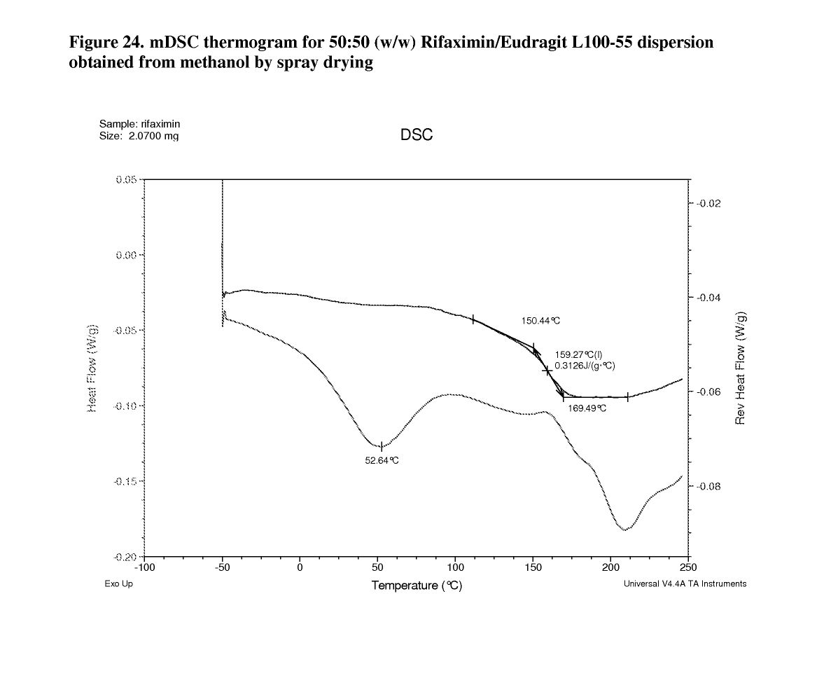

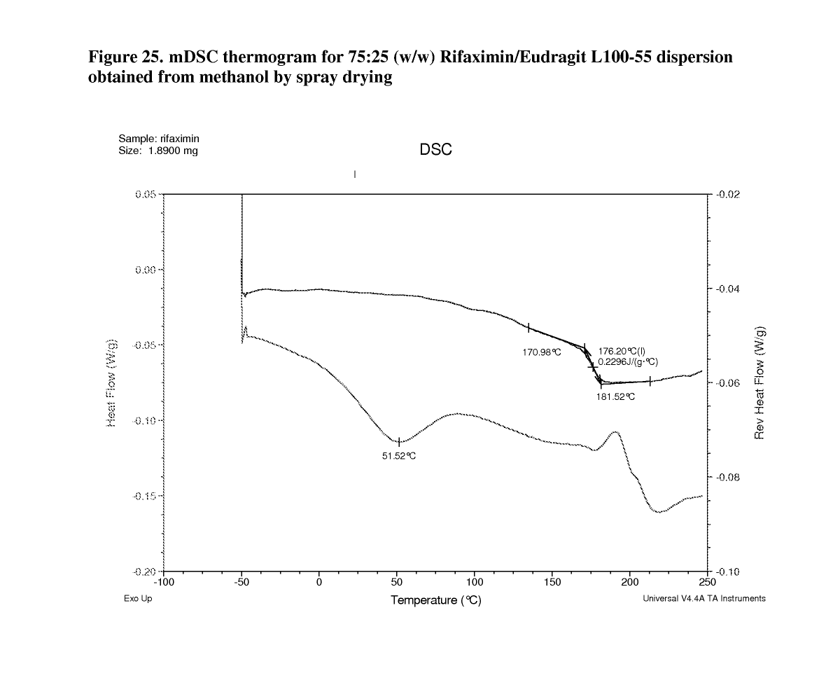

Dispersions prepared with Eudragit L100-55 exhibit Tg at 141° C. with ΔCp approximately 0.5 J/g·° C. (FIG. 23, 25:75 w/w), 159° C. with ΔCp approximately 0.3 J/g·° C. (FIG. 24, 50:50 w/w), and 176° C. with ΔCp at Tg approximately 0.2 J/g·° C. (FIG. 25, 75:25 w/w) respectively.

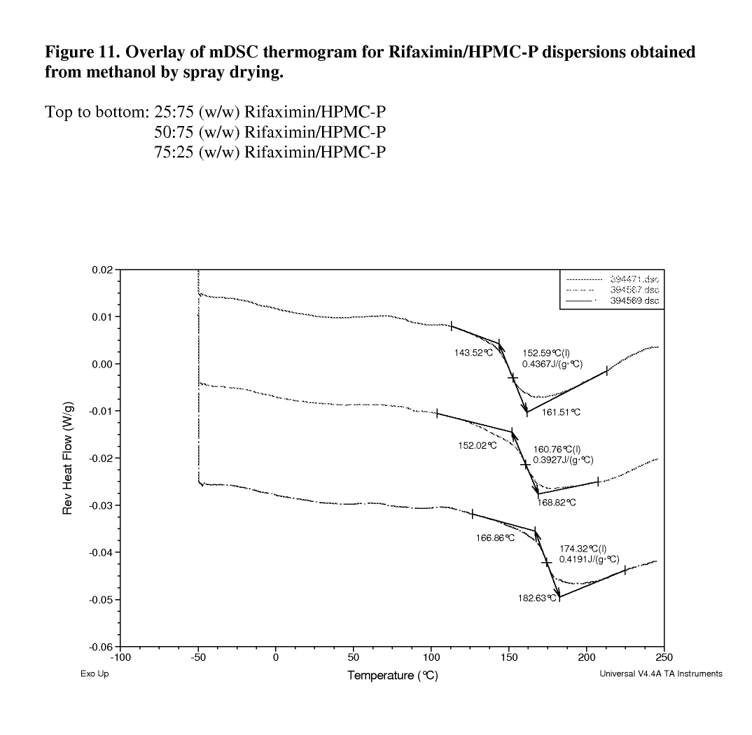

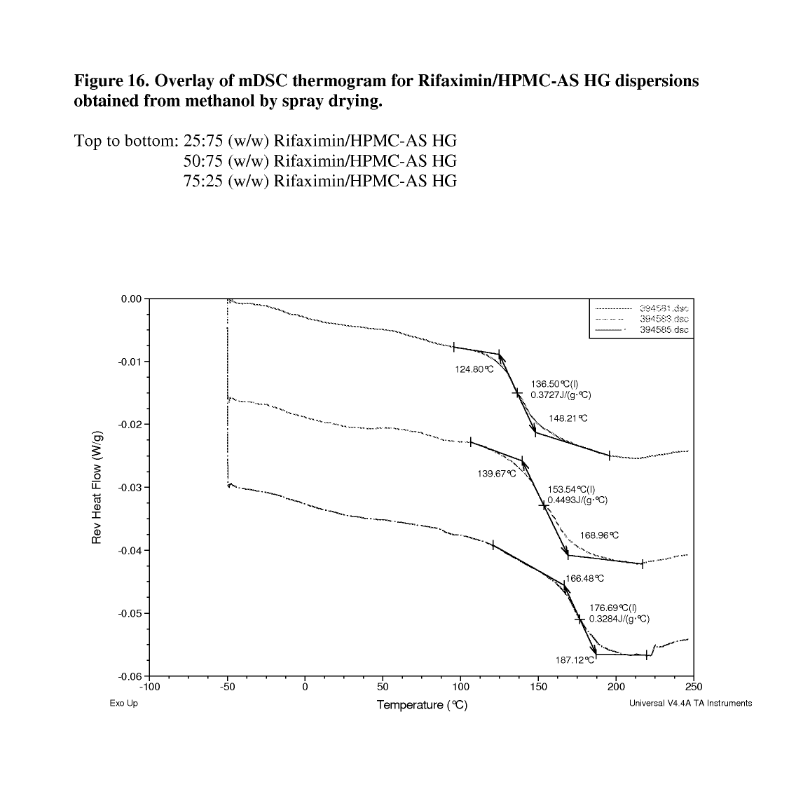

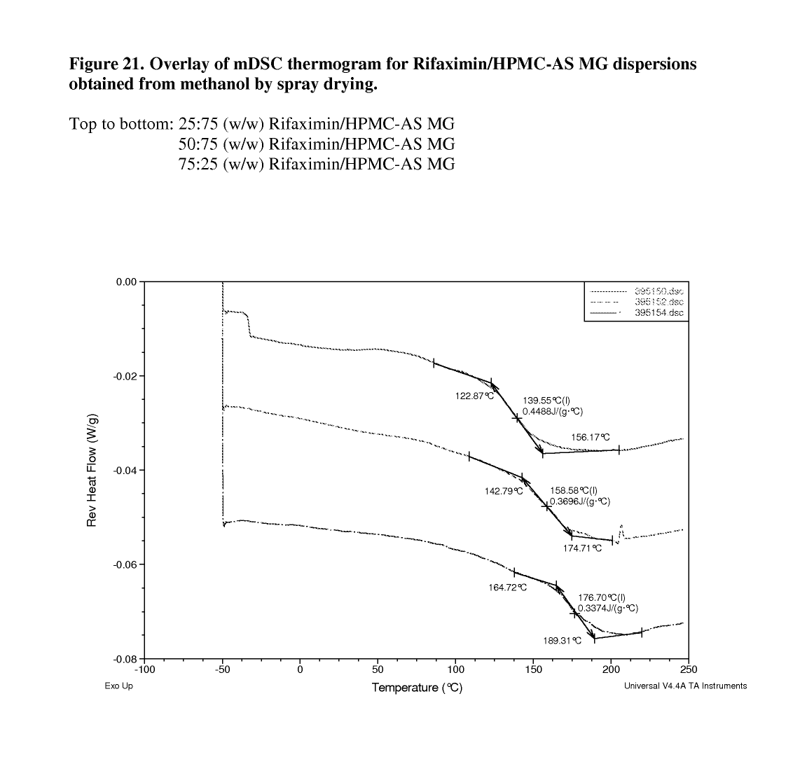

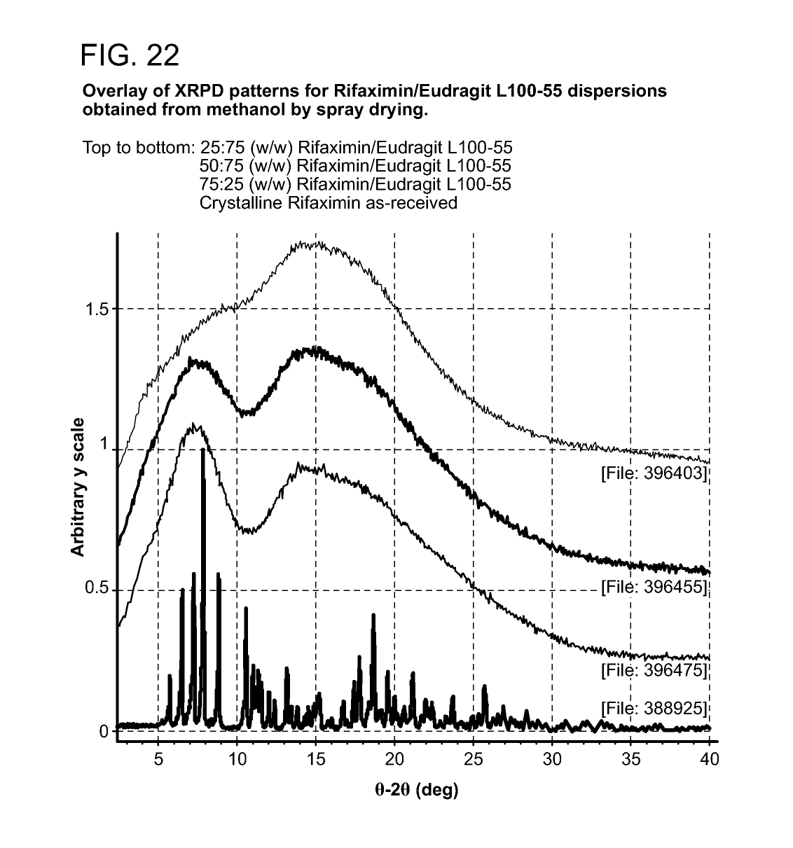

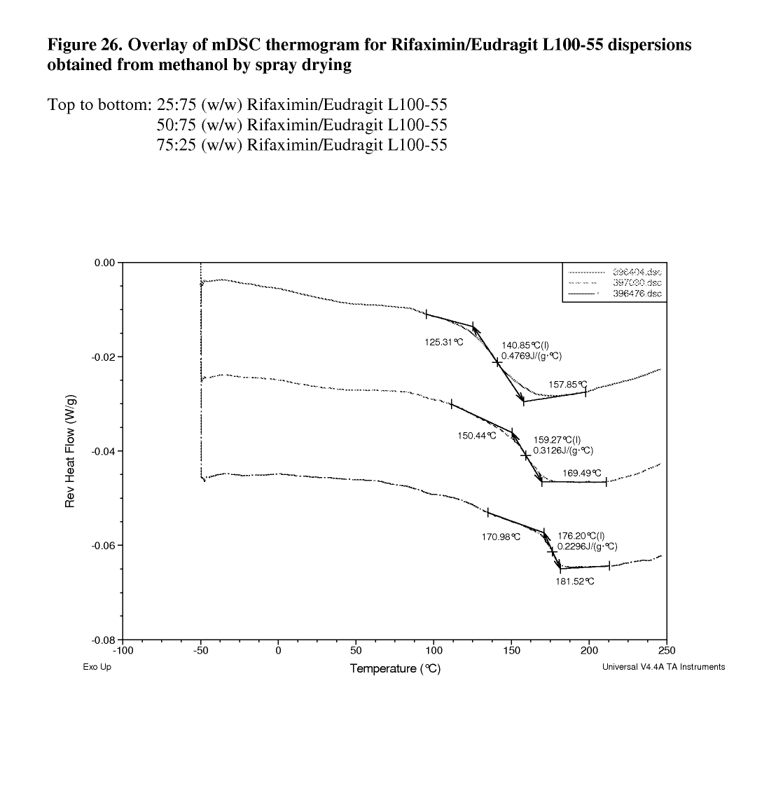

Similarly, as shown in FIG. 11 (with HPMC-P), FIG. 16 (with HPMC-AS HG), FIG. 21 (with HPMC-AS MG, and FIG. 26 (with Eudragit L100-55), Tg of material in each set of Rifaximin/polymer dispersions increases with the increased Rifaximin concentration due to the higher Tg of Rifaximin.

### Physical Stability Assessment

An assessment of physical stability for rifaximin/polymer dispersions was conducted under stress conditions of aqueous solutions at different biologically relevant conditions, including 0.1N HCl solution at 37° C. and pH 6.5 FASSIF buffer at 37° C., elevated temperature/relative humidity (40° C./75% RH), and elevated temperature/dry (60° C.). The x-ray amorphous rifaximin—only sample prepared from methanol by spray drying was also stressed under the same conditions for comparison.

### Stress in 0.1N HCl Solution at 37° C.

For the assessment of physical stability for samples in a 0.1N HCl solution maintained at 37° C., observations were made and microscopy images were acquired using polarized light at different time points including 0, 6 and 24 hrs, as summarized in Table 6. Based on the absence of birefringent particles when samples were observed by PLM, dispersions prepared with HPMC-AS HG and HPMC-AS MG display the highest physical stability under this particular stress condition. The results of this study for each of samples are discussed below.

X-ray amorphous Rifaximin stressed in 0.1N HCl solution at 37° C. at 0, 6, and 24 hrs showed evidence of birefringence/extinctions was observed at 6 hrs, indicating the occurrence of devitrification of the material.

Samples at compositions of 25:75 and 50:50 (w/w) crystallized at 6 hrs; sample at 75:25 (w/w) composition crystallized within 24 hrs while no evidence of crystallization was observed at 6 hrs or earlier. The decreased stability of Rifaximin/PVP K-90 dispersions in 0.1N HCl solution with increased PVP K-90 concentration may due to the high solubility of PVP K-90 in the solution.

Irregular aggregates without birefringence/extinctions were observed for dispersion prepared with HPMC-P at t=0 hr, the initial time point when 0.1N HCl solution was just added into solids. After 24 hrs, samples at compositions of 25:75 and 50:50 (w/w) remained as non-birefringent aggregates, indicating no occurrence of devitrification under the conditions examined. Evidence of crystallization was observed for sample of 75:25 (w/w) composition at 6 hrs. No birefringence/extinctions were observed for all of dispersions prepared with HPMC-AS HG and HPMC-AS MG after 24 hrs, suggesting these samples are resistant to devitrification upon exposure to 0.1N HCl solution for 24 hrs.

For dispersions prepared with Eudragit L100-55, upon exposure to 0.1N HCl solution for 24 hrs, birefringent particles with extinctions were observed only in the sample at 50:50 (w/w) composition. Considered that no evidence of crystallization was observed for dispersions of compositions at 25:75 and 75:25 (w/w), it is unknown whether such birefringence was caused by some foreign materials or by crystalline solids indicating the occurrence of devitrification.

### Stress in pH 6.5 FASSIF Buffer at 37° C.

An assessment of physical stability of dispersions prepared was also performed in pH 6.5 FASSIF buffer maintained at 37° C. X-ray amorphous Rifaximin material was also stressed under same condition for comparison. PLM observations indicated that dispersions prepared from HPMC-AS HG and HPMC-AS MG display the highest physical stability under this stress condition. X-ray amorphous rifaximin-only material crystallized within 6 hrs, so did all rifaximin/PVP K-90 dispersions. For dispersions prepared with HPMC-P, birefringent particles with extinctions were observed in samples at 50:50 and 75:25 (w/w) compositions within 6 hrs, indicating the occurrence of devitrification in materials. No evidence of any birefringence/extinctions was observed in 25:75 (w/w) rifaximin/HPMC-P dispersion material after 24 hrs. No birefringence/extinctions were observed for all of dispersions prepared with HPMC-AS HG and HPMC-AS MG after 24 hrs, suggesting these samples are resistant to devitrification upon exposure to pH 6.5 FASSIF buffer for 24 hrs. Rifaximin/Eudragit L100-55 dispersions at 50:50 and 75:25 (w/w) compositions crystallized with 6 hrs while no evidence of crystallization was observed in the sample at 25:75 (w/w) composition after 24 hrs.

### Stress at 40° C./75% RH Condition

The samples including all the dispersions and x-ray amorphous rifaximin-only material were assessed for evidence of crystallization based on observations by microscopy using polarized light. Each of the samples remained as irregular aggregates without birefringence/extinctions after stressed at 40° C./75% RH condition for 7 days.

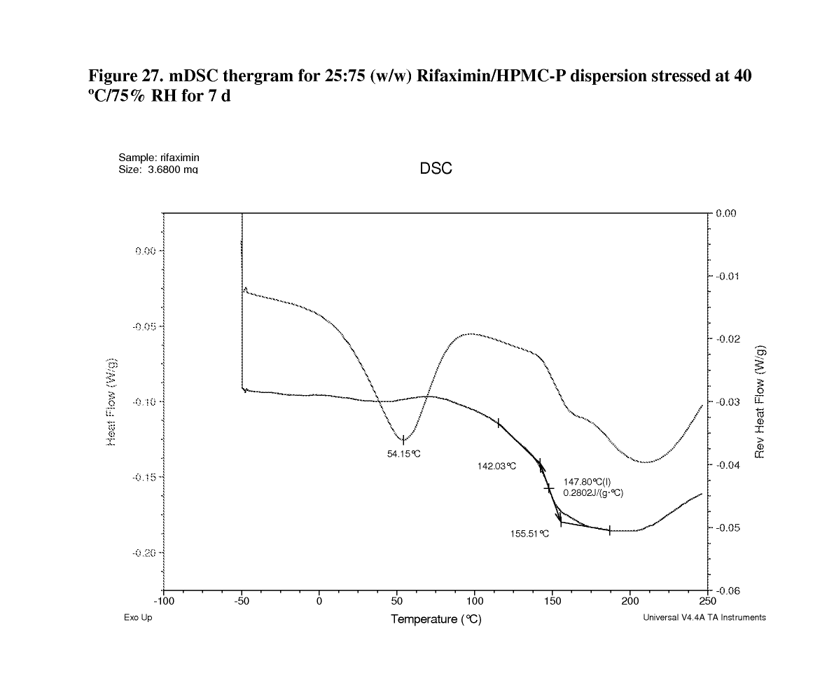

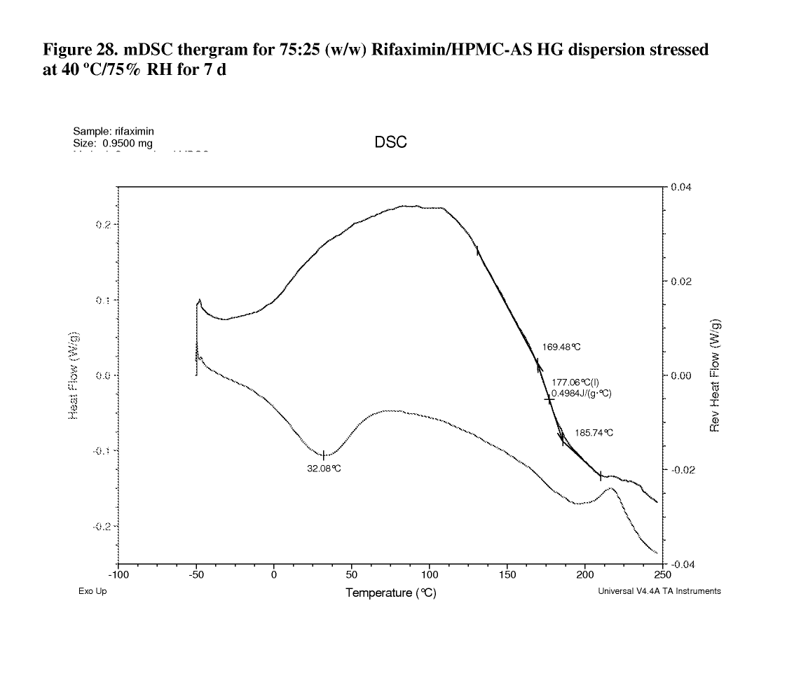

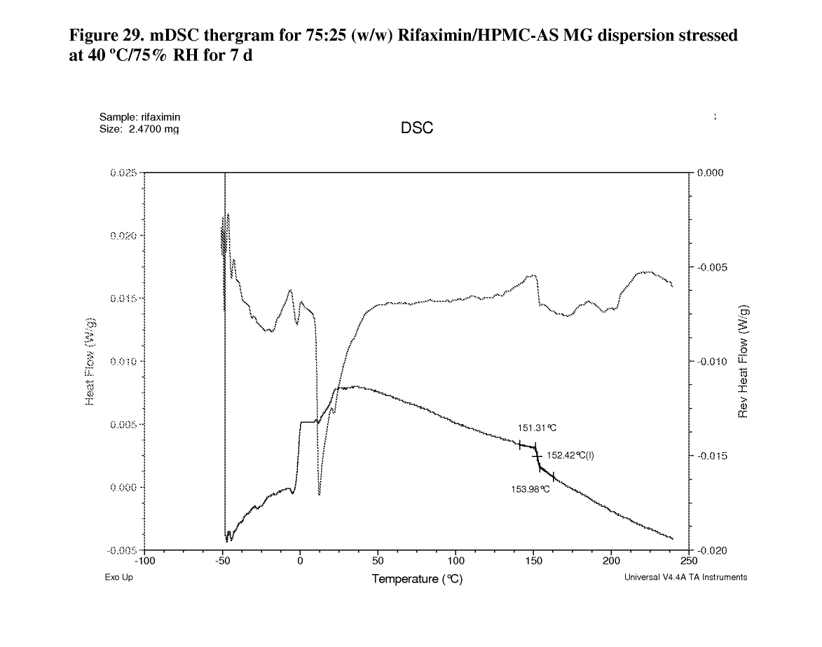

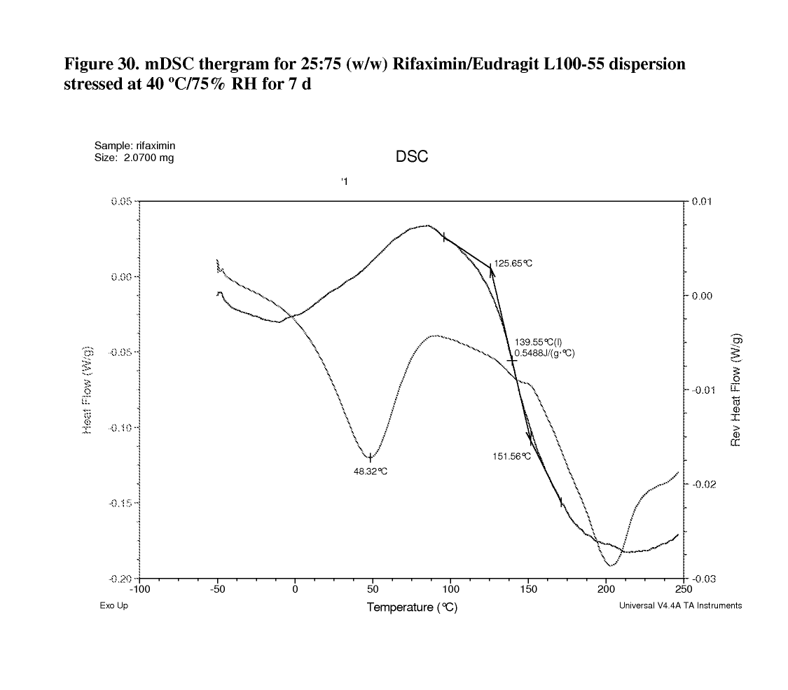

Modulated DSC analyses were carried out on selected samples including 25:75 (w/w) rifaximin/HPMC-P, 75:25 (w/w) rifaximin/HPMC-AS HG, 75:25 (w/w) rifaximin/HPMC-AS MG, and 25:75 (w/w) Rifaximin/Eudragit L100-55 to inspect for evidence of phase separation after exposure to 40° C./75% RH for 7 days. All of samples display a single apparent Tg at approximately 148° C. (FIG. 27, 25:75 (w/w) HPMC-P), 177° C. (FIG. 28, 75:25 (w/w) HPMC-AS HG) 152° C. (FIG. 29, 75:25 (w/w) HPMC-AS MG) and 140° C. (FIG. 30, 25:75 (w/w) Eudragit L100-55) respectively, indicating the components of each dispersion remained intimately miscible after stress. Although crimped with manual pin-hole DSC pan was used, the release of moisture from sample upon heating can still be observed from non-reversible heat flow signals.

### Stress at 60° C./Dry Condition

All the dispersions and x-ray amorphous rifaximin-only material were also stressed at 60° C./dry condition for 7 days and were assessed for evidence of crystallization based on observations by microscopy using polarized light. Each of the samples remained as irregular aggregates without birefringence/extinctions after stressed at this condition for 7 days.

### Rifaximin Solid Dispersions by Spray Drying

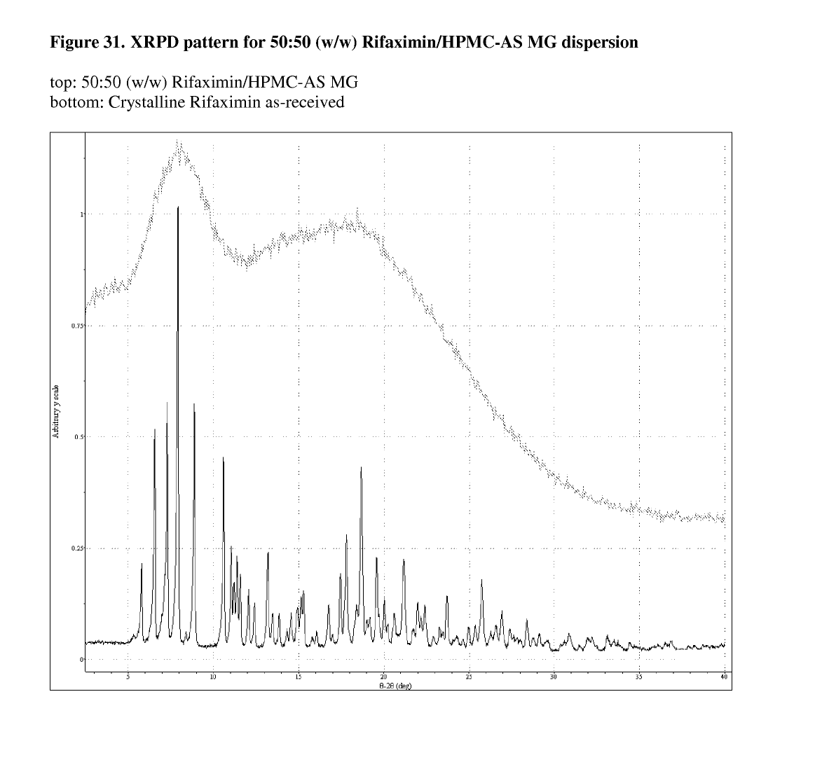

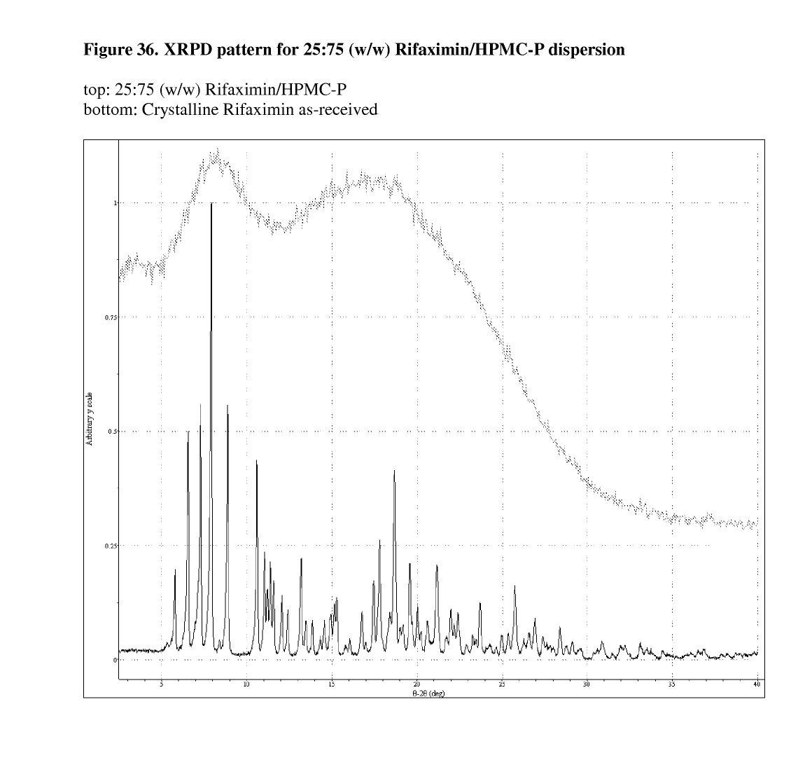

Based on the experimental results from screen, HPMC-AS MG and HPMC-P were used to prepare additional quantities of solid dispersions at gram-scale by spray drying. The operating parameters used for processing are presented in Table 9. Based on visual inspection, both dispersions were x-ray amorphous by XRPD (FIG. 31 and FIG. 36).

### Characterization of 50:50 (w/w) Rifaximin/HPMC-AS MG Dispersion

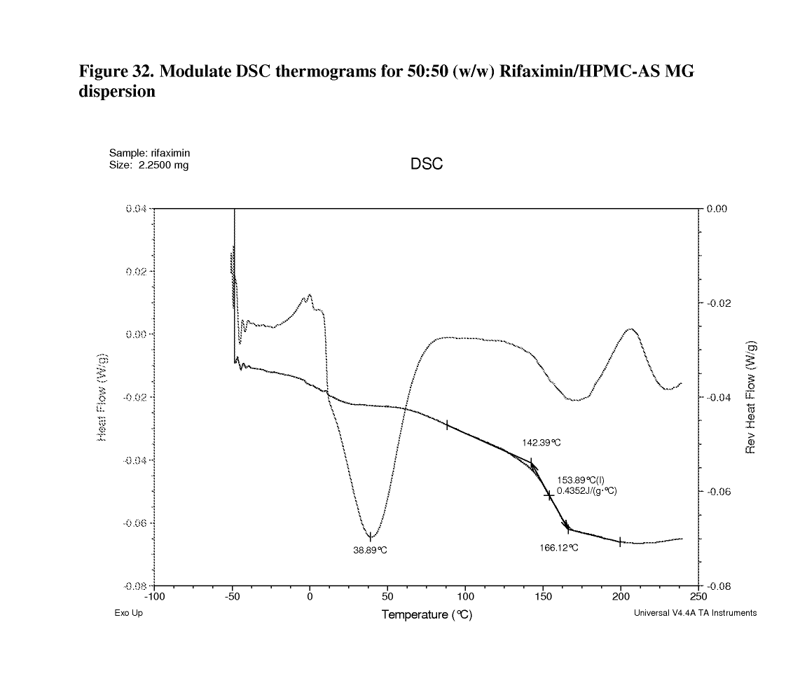

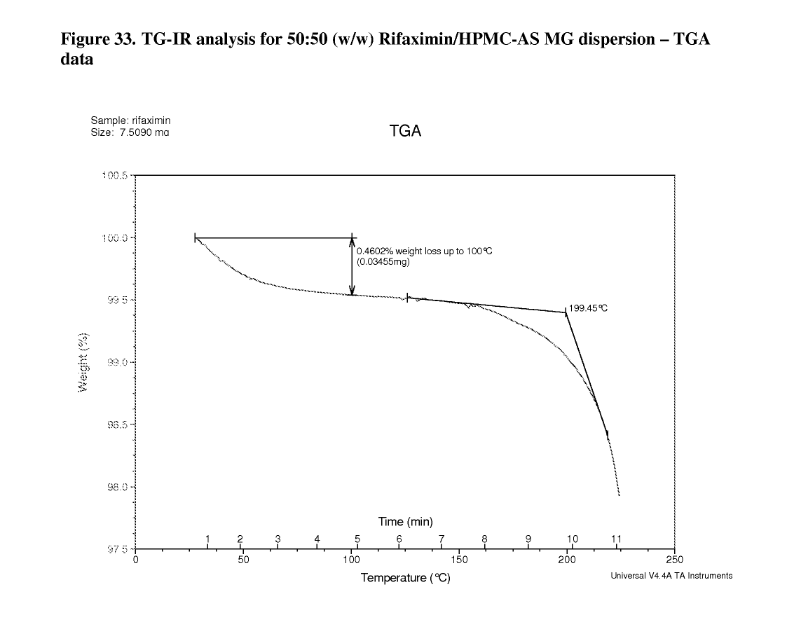

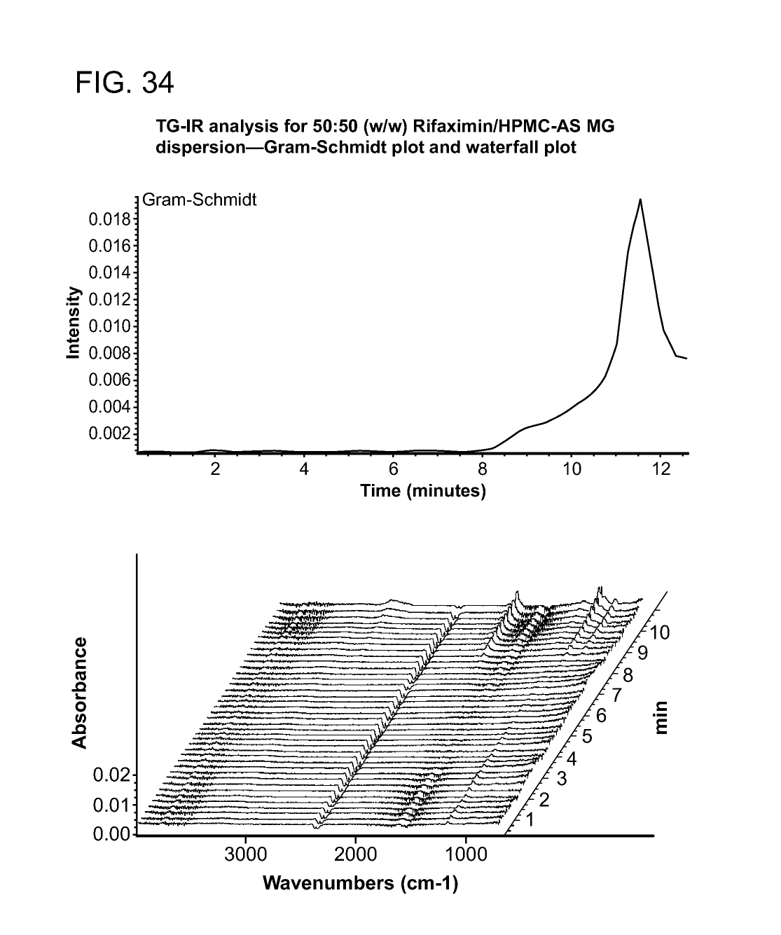

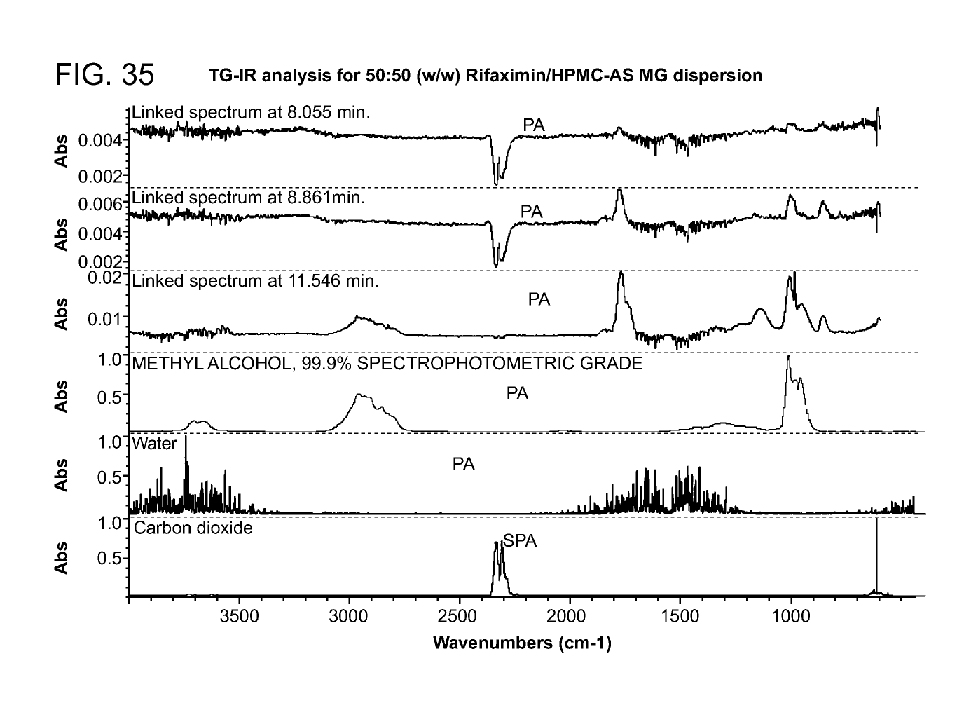

Characterization and results for the 50% API loading HPMC-AS MG are summarized in Table 10. The sample was x-ray amorphous based on high resolution XRPD. A single Tg at approximately 154° C. was observed from the apparent step change in the reversing heat flow signal in mDSC with the change of heat capacity 0.4 J/g ° C. A non-reversible endotherm was observed at approximately 39° C. which is likely due to the residual solvent in the materials (FIG. 32). TG-IR analysis was carried out in order to determine volatile content on heating. TGA data for this material is shown in FIG. 34. There was a 0.5% weight loss up to ˜100° C. A Gram-Schmidt plot corresponding to the overall IR intensity associated with volatiles released by solids upon heating at 20° C./min is shown in FIG. 33. There was a dramatic increase of intensity of released volatiles after ˜8 minutes, with a maximum at ˜11.5 minutes. The waterfall plot (FIG. 34) and the linked IR spectrum (FIG. 35) are indicative of the loss of water loss up to ˜8 minutes then methanol and some unknown volatiles thereafter. This is consistent with the dramatic change in the slope in the TGA and may indicate decomposition of material.

### Characterization of 25:75 (w/w) Rifaximin/HPMC-P Dispersion

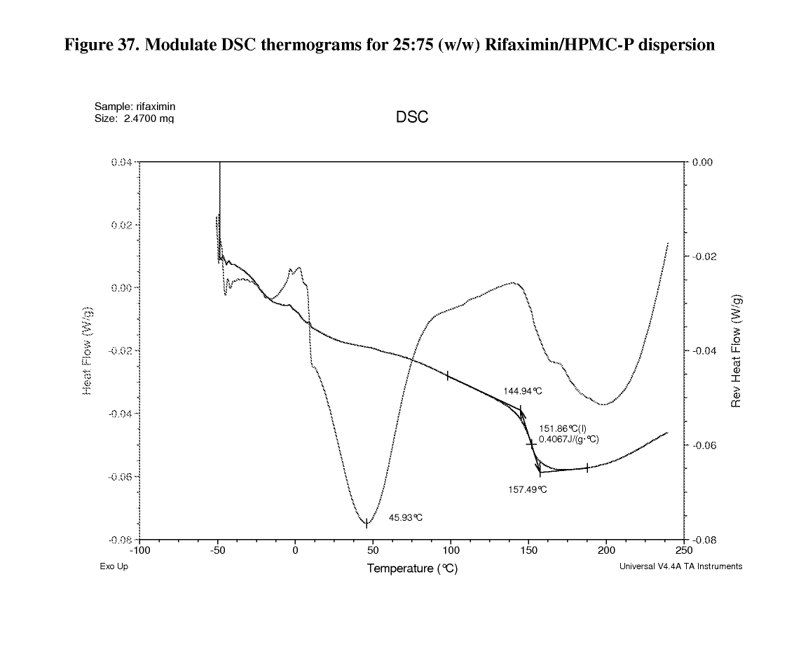

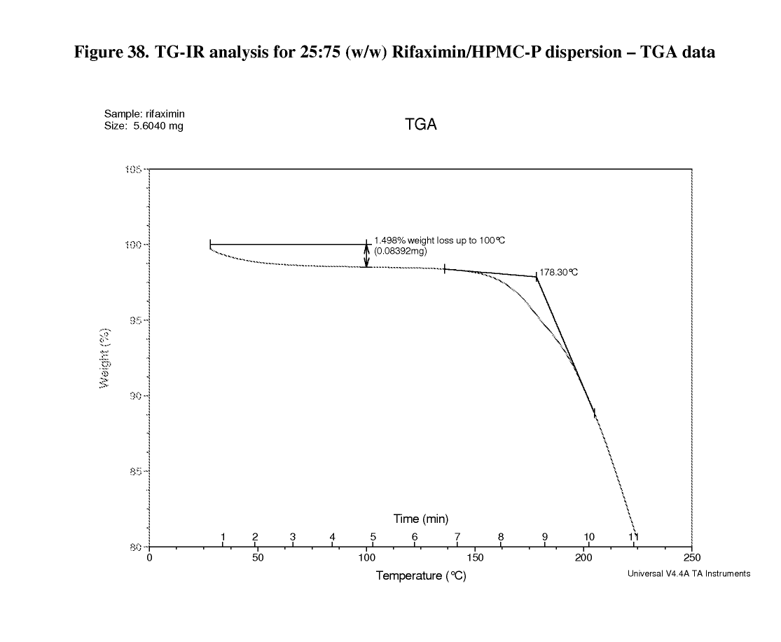

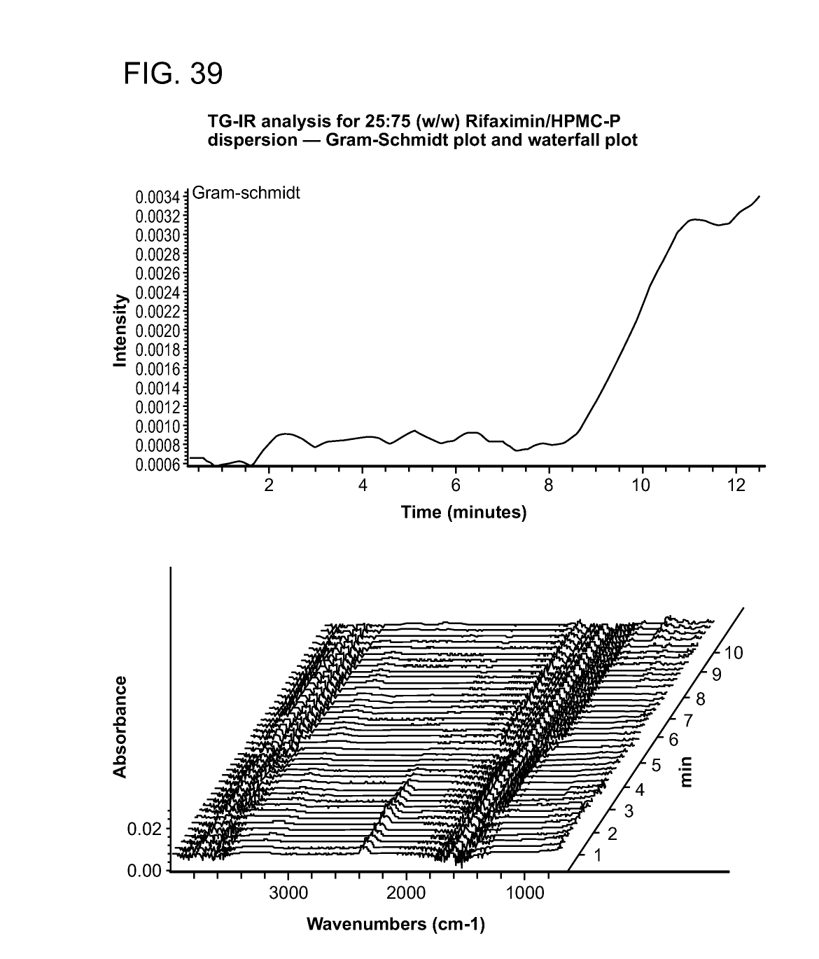

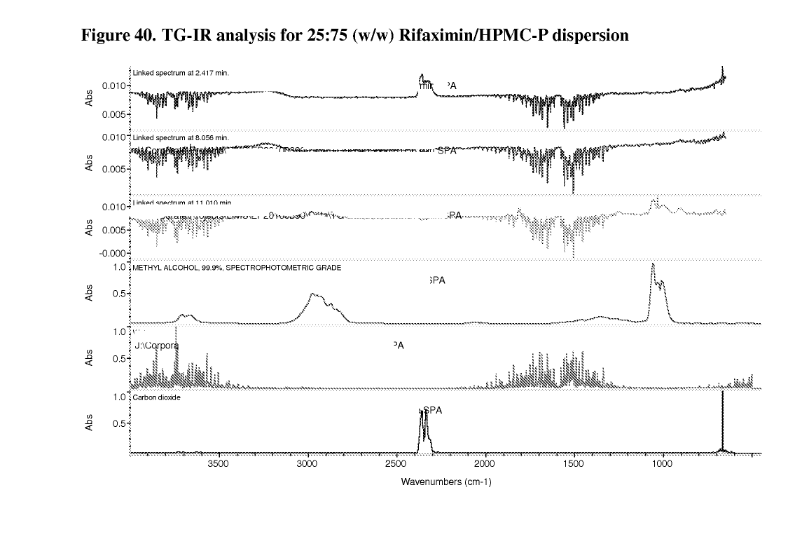

Characterization and results for the 25% API loading dispersion of HPMC-P are summarized in Table 11. Solids were x-ray amorphous based on high resolution XRPD (FIG. 36). By mDSC, there is a single Tg at approximately 152° C. from the apparent step change in the reversing heat flow signal. The change of heat capacity is 0.4 J/g ° C. (FIG. 37). A non-reversible endotherm, which is likely due to the residual solvent in the materials, was observed at approximately 46° C. Volatiles generated on heating were analyzed by TG-IR. The total weight loss of sample was approximately 1.5 wt % to 100° C. and the dramatic change in the slope occurs at approximately 178° C. (FIG. 38). The Gram-Schmidt plot (FIG. 39) shows a small increase of intensity upon heating after ˜2 minutes, followed by negligible change of intensity until ˜9 minutes. Then dramatic change of intensity can be observed with a maximum at ˜11 minutes, followed by a final increase of intensity above ˜12 minutes. As seen in the waterfall plot (FIG. 39), some volatiles were released during entire heating period (data is shown in FIG. 40 using the linked IR spectrum at different time points as an example). The sample released water during entire heating period and methanol after ˜9 minutes.

### Dispersions Miscibility Study by Multivariate Mixture Analysis

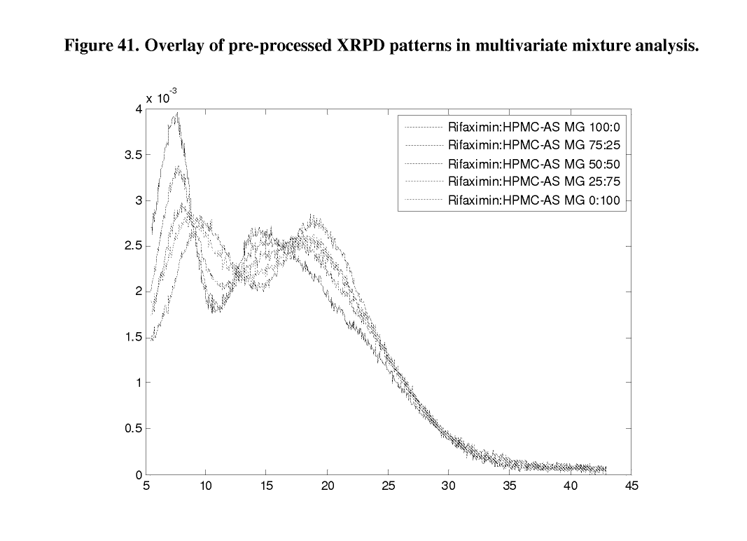

For Rifaximin/HPMC-AS MG dispersions prepared by spray drying, a multivariate mixture analysis was performed using the XRPD data to examine the physical state of the components and inspect for evidence of miscibility. The analysis was done with MATLAB (v7.6.0) and Unscrambler (v 9.8) and it was not performed under cGMP guidelines. XRPD patterns of all the samples were truncated with their baseline corrected, and unit area normalized before analysis. The pre-possessed XRPD patterns are shown in FIG. 41.

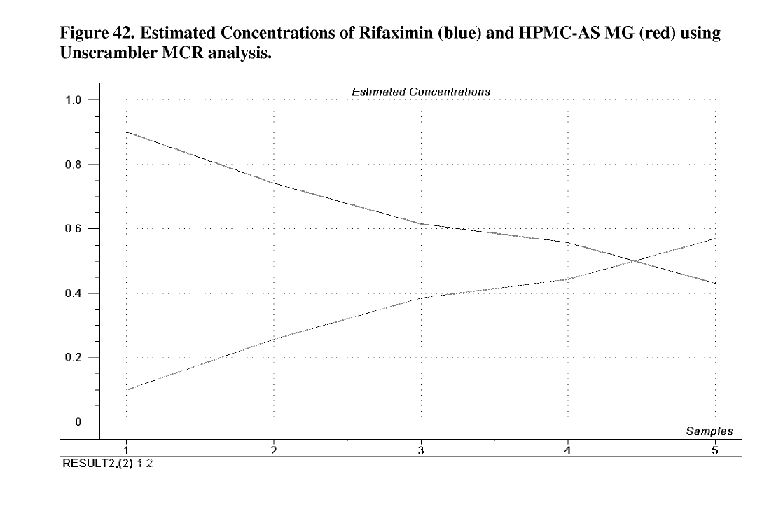

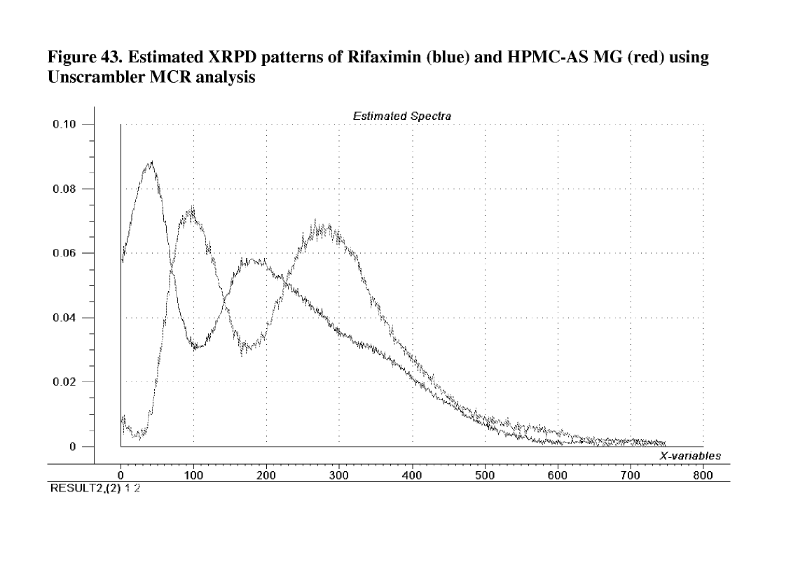

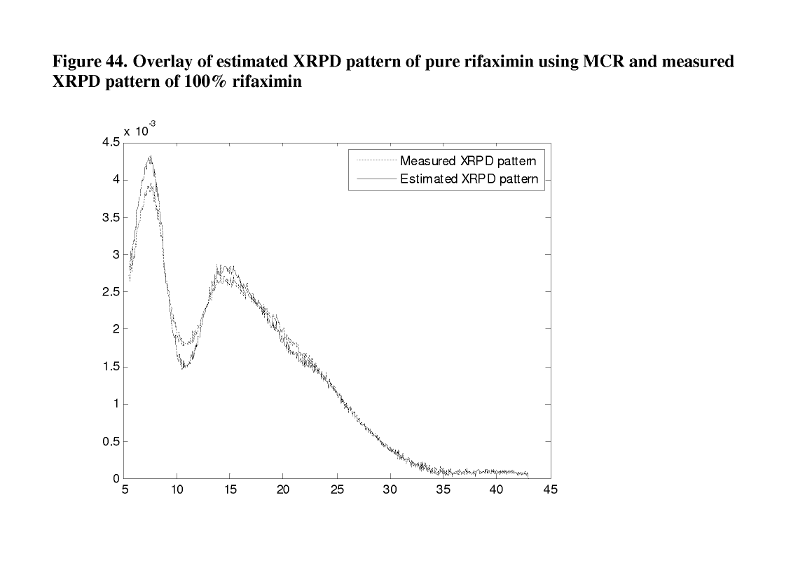

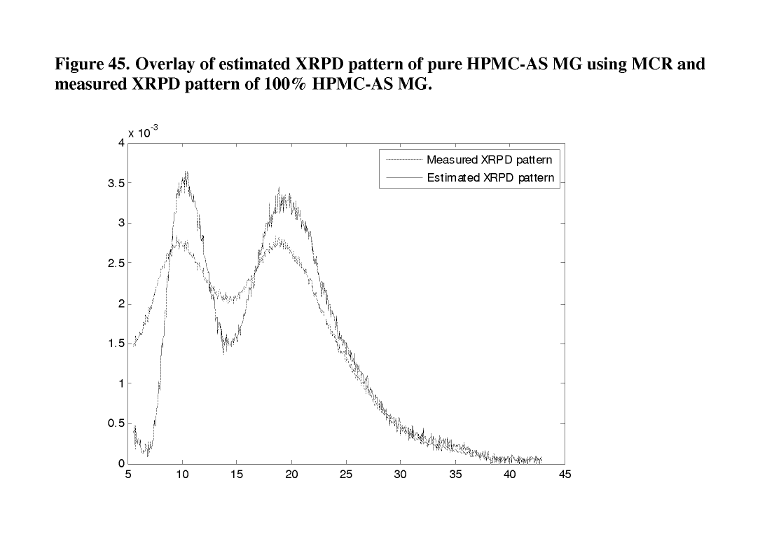

In the analysis, Rifaximin and HPMC-AS MG were assumed to be separated phases (no miscibility) and the compositions of Rifaximin and HPMC-AS MG in each sample were estimated based on this assumption. As shown in FIG. 42, the estimated ratios of Rifaximin to HPMC-AS MG based on pure separated phases did not agree with samples actual compositions, especially for the samples with high compositions of HPMC-AS MG (low Rifaximin loading). Also, the calculated XRPD patterns for Rifaximin and HMPC-AS MG based on the assumption of separated phases (FIG. 43) compared to actual experimental XRPD patterns for Rifaximin (FIG. 44) and HPMC-AS MG (FIG. 45) were generated. Although the calculated Rifaximin pattern is similar to its experimental pattern, the calculated HMPC-AS MG pattern is quite different from its experimental pattern. Both results suggest that Rifaximin and HPMC-AS MG are not separated phases but miscible in the dispersions. The differences in the estimated and actual compositions are likely due to the interaction between Rifaximin and HPMC-AS MG.

TABLE 1
Solid Dispersion Attempts for Rifaximin/PVP
K-90 by Spray Drying

| Description (a, b) | Habit/Description | Analysis | Result (c) |
|---|---|---|---|
| (25:75) | solids orange; | XRPD | x-ray amorph. |
| PVP K-90 | aggregates, | mDSC | 185° C. (T_{g}, |
| | irregular, | | midpoint); |
| | no B/E | | 0.3 J/g · ° C. (ΔCₚ) |
| (50:50) | solids orange; | XRPD | x-ray amorph. |
| PVP K-90 | aggregates, | mDSC | 193° C. (T_{g}, |
| | irregular, | | midpoint); |
| | no B/E | | 0.3 J/g · ° C. (ΔCₚ) |
| (75:25) | solids orange; | XRPD | x-ray amorph. |
| PVP K-90 | aggregates, | mDSC | 197° C. (T_{g}, |
| | irregular, | | midpoint); |
| | no B/E | | 0.3 J/g · ° C. (ΔCₚ) |

(a): approximate ratio of Rifaximin to polymer, by weight;
(b): samples stored in freezer over desiccant after prepared.

TABLE 2
Solid Dispersion Attempts for Rifaximin/HPMC-P
by Spray Drying

| Description (a, b) | Habit/Description | Analysis | Result (c) |
|---|---|---|---|
| (25:75) | solids light | XRPD | x-ray amorph. |
| HPMC-P | orange; | mDSC | 153° C. (T_{g}, |
| | aggregates, | | midpoint); |
| | irregular, | | 0.4 J/g · ° C. (ΔCₚ) |
| | no B/E | | |
| (50:50) | solids orange; | XRPD | x-ray amorph. |
| HPMC-P | aggregates, | mDSC | 161° C. (T_{g}, |
| | irregular, | | midpoint); |
| | no B/E | | 0.4 J/g · ° C. (ΔCₚ) |
| (75:25) | solids orange; | XRPD | x-ray amorph. |
| HPMC-P | aggregates, | mDSC | 174° C. (T_{g}, |
| | irregular, | | midpoint); |
| | no B/E | | 0.4 J/g · ° C. (ΔCₚ) |

(a): approximate ratio of Rifaximin to polymer, by weight;
(b): samples stored in freezer over desiccant after prepared.

TABLE 3
Solid Dispersion Attempts for Rifaximin/HPMC-AS
HG by Spray Drying

| Description (a, b) | Habit/Description | Analysis | Result (c) |
|---|---|---|---|
| (25:75) | solids light | XRPD | x-ray amorph. |
| HPMC-AS HG | orange; | mDSC | 137° C. (T_{g}, |
| | aggregates, | | midpoint); |
| | irregular, | | 0.4 J/g · ° C. (ΔCₚ) |
| | no B/E | | |
| (50:50) | solids orange; | XRPD | x-ray amorph. |
| HPMC-AS HG | aggregates, | mDSC | 154° C. (T_{g}, |
| | irregular, | | midpoint); |
| | no B/E | | 0.4 J/g · ° C. (ΔCₚ) |
| (75:25) | solids orange; | XRPD | x-ray amorph. |
| HPMC-AS HG | aggregates, | mDSC | 177° C. (T_{g}, |
| | irregular, | | midpoint); |
| | no B/E | | 0.3 J/g · ° C. (ΔCₚ) |

(a): approximate ratio of Rifaximin to polymer, by weight;
(b): samples stored in freezer over desiccant after prepared.

TABLE 4
Solid Dispersion Attempts for Rifaximin/HPMC-AS
MG by Spray Drying

| Description (a, b) | Habit/Description | Analysis | Result (c) |
|---|---|---|---|
| (25:75) | solids light | XRPD | x-ray amorph. |
| HPMC-AS MG | orange; | mDSC | 140° C. (T_{g}, |
| | aggregates, | | midpoint); |
| | irregular, | | 0.4 J/g · ° C. (ΔCₚ) |
| | no B/E | | |
| (50:50) | solids orange; | XRPD | x-ray amorph. |
| HPMC-AS MG | aggregates, | mDSC | 159° C. (T_{g}, |
| | irregular, | | midpoint); |
| | no B/E | | 0.4 J/g · ° C. (ΔCₚ) |
| (75:25) | solids orange; | XRPD | x-ray amorph. |
| HPMC-AS MG | aggregates, | mDSC | 177° C. (T_{g}, |
| | irregular, | | midpoint); |
| | no B/E | | 0.3 J/g · ° C. (ΔCₚ) |

(a): approximate ratio of Rifaximin to polymer, by weight;
(b): samples stored in freezer over desiccant after prepared.

TABLE 5
Solid Dispersion Attempts for Rifaximin/Eudragit
L100-55 by Spray Drying

| Description (a, b) | Habit/Description | Analysis | Result (c) |
|---|---|---|---|
| (25:75) | solids light | XRPD | x-ray amorph. |
| Eudragit | orange; | mDSC | 141° C. (T_{g}, |
| L100-55 | aggregates, | | midpoint); |
| | irregular, | | 0.5 J/g · ° C. (ΔCₚ) |
| | no B/E | | |
| (50:50) | solids orange; | XRPD | x-ray amorph. |
| Eudragit | aggregates, | mDSC | 159° C. (T_{g}, |
| L100-55 | irregular, | | midpoint); |
| | no B/E | | 0.3 J/g · ° C. (ΔCₚ) |
| (75:25) | solids orange; | XRPD | x-ray amorph. |
| Eudragit | aggregates, | mDSC | 176° C. (T_{g}, |
| L100-55 | irregular, | | midpoint); |
| | no B/E | | 0.2 J/g · ° C. (ΔCₚ) |

(a): approximate ratio of Rifaximin to polymer, by weight;
(b): samples stored in freezer over desiccant after prepared.

TABLE 6
Physical Stability Assessment in 0.1N HCl at 37° C. for Rifaximin
and Rifaximin Dispersions Prepared in Methanol by Spray Drying

| Description (a) | Time (b) | Habit/Description | Analysis | Results |
|---|---|---|---|---|
| (100:0) | 0 | — | PLM | agg., irr., no B/E |
| Rifaximin- | | | | agg., irr., no B/E |
| only | 6 hrs | orange solids, no | PLM | agg., no B/E + a few B/E particles |
| | | liquid left | | clear view of B/E particles |
| | 24 hrs | orange solids, | PLM | agg., no B/E + a few B/E particles |
| | | solution cloudy | | |
| (25:75) | 0 | — | PLM | agg., irr., no B/E |
| PVP K-90 | | | | agg., irr., no B/E |
| | 6 hrs | orange solids, | PLM | agg., no B/E + B/E particles |
| | | solution slightly | | clear view of B/E particles |
| | | yellow | | |
| | 24 hrs | orange solids, | PLM | agg., no B/E + B/E particles |
| | | solution slightly | | |
| | | yellow | | |
| (50:50) | 0 | — | PLM | agg., irr., no B/E |
| PVP K-90 | | | | agg., irr., no B/E |
| | 6 hrs | orange solids, | PLM | agg., no B/E + a few B/E particles |
| | | solution slightly | | clear view of B/E particles |
| | | yellow | | |
| | 24 hrs | orange solids, small | PLM | majority agg., no B/E + a few B/E |
| | | amount of liquid | | particles |
| | | left | | clear view of B/E particles |
| (75:25) | 0 | — | PLM | agg., irr., no B/E |
| PVP K-90 | | | | agg., irr., no B/E |
| | 6 hrs | orange solids, | PLM | agg., no B/E |
| | | solution slightly | | agg., no B/E |
| | | yellow | | |
| | 24 hrs | orange solids, small | PLM | agg., no B/E |
| | | amount of liquid | | a few B/E particles in view field |
| | | left | | |
| (25:75) | 0 | — | PLM | agg., irr., no B/E |
| HPMC-P | | | | agg., irr., no B/E |
| | 6 hrs | light orange solids, | PLM | agg., no B/E |
| | | liquid turbid | | agg., no B/E |
| | 24 hrs | orange solids, liquid | PLM | agg., no B/E |
| | | turbid | | agg., no B/E |
| (50:50) | 0 | — | PLM | agg., irr., no B/E |
| HPMC-P | | | | agg., irr., no B/E |
| | 6 hrs | orange solids, liquid | PLM | agg., no B/E |
| | | turbid | | agg., no B/E |
| | 24 hrs | orange solids, | PLM | agg., no B/E |
| | | solution cloudy | | agg., no B/E |
| (75:25) | 0 | — | PLM | agg., irr., no B/E |
| HPMC-P | | | | agg., irr., no B/E |
| | 6 hrs | orange solids, liquid | PLM | agg., no B/E + some B/E particles |
| | | turbid | | clear view of B/E particles |
| | 24 hrs | orange solids, small | PLM | B/E particles observed |
| | | amount of liquid | | clear view of B/E particles |
| | | left | | |
| (25:75) | 0 | — | PLM | agg., irr., no B/E |
| HPMC-AS HG | | | | agg., irr., no B/E |
| | 6 hrs | light orange solids | PLM | no B/E observed |
| | | in cloudy liquid | | no B/E observed |
| | 24 hrs | orange solids in | PLM | no B/E observed |
| | | cloudy solution | | no B/E observed |
| (50:50) | 0 | — | PLM | agg., irr., no B/E |
| HPMC-AS HG | | | | agg., irr., no B/E |
| | 6 hrs | orange solids, liquid | PLM | no B/E observed |
| | | cloudy | | no B/E observed |
| | 24 hrs | orange solids in | PLM | no B/E observed |
| | | cloudy solution | | |
| (75:25) | 0 | — | PLM | agg., irr., no B/E |
| HPMC-AS HG | | | | agg., irr., no B/E |
| | 6 hrs | orange solids, liquid | PLM | no B/E observed |
| | | turbid | | no B/E observed |
| | 24 hrs | orange solids + | PLM | agg., no B/E |
| | | cloudy solution | | agg., no B/E |
| (25:75) | 0 | — | PLM | agg., irr., no B/E |
| HPMC-AS MG | | | | agg., irr., no B/E |
| | 6 hrs | light orange solids | PLM | no B/E observed |
| | | in cloudy liquid | | |
| | 24 hrs | orange solids in | PLM | majority no B/E, a few B/E particles |
| | | cloudy liquid | | B/E particles seems fiber-like, may |
| | | | | due to foreign materials |
| (50:50) | 0 | — | PLM | agg., irr., no B/E |
| HPMC-AS MG | | | | agg., irr., no B/E |
| | 6 hrs | orange solids, | PLM | agg., no B/E + a few B/E particles |
| | | liquid turbid | | seems due to foreign material |
| | | | | clear view of B/E particles |
| | 24 hrs | orange solids in | PLM | no B/E observed |
| | | cloudy solution | | no B/E observed |
| (75:25) | 0 | — | PLM | agg., irr., no B/E |
| HPMC-AS MG | | | | agg., irr., no B/E |
| | 6 hrs | orange solids, | PLM | no B/E observed |
| | | liquid turbid | | no B/E observed |
| | 24 hrs | orange solids in | PLM | no B/E observed |
| | | cloudy liquid | | agg., no B/E |
| (25:75) | 0 | — | PLM | agg., irr., no B/E |
| Eudragit | | | | agg., irr., no B/E |
| L100-55 | 6 hrs | light orange solids | PLM | no B/E observed |
| | | in cloudy liquid | | |
| | 24 hrs | orange solids in | PLM | no B/E observed |
| | | cloudy solution | | |
| (50:50) | 0 | — | PLM | agg., irr., no B/E |
| Eudragit | | | | agg., irr., no B/E |
| L100-55 | 6 hrs | orange solids in | PLM | no B/E observed except 2 particles |
| | | cloudy liquid | | |
| | 24 hrs | orange solids in | PLM | majority no B/E, a few B/E particles |
| | | cloudy solurion | | in center |
| | | | | clear view of B/E particles |
| (75:25) | 0 | — | PLM | agg., irr., no B/E |
| Eudragit | | | | agg., irr., no B/E |
| L100-55 | 6 hrs | orange solids, | PLM | agg., no B/E |
| | | liquid turbid | | agg., no B/E |
| | 24 hrs | orange solids in | PLM | agg., no B/E |
| | | cloudy liquid | | |

(a): approximate ratio of Rifaximin to polymer, by weight.
(b): time is cumulative and approximate; 100 μL of 0.1N HCl solution added into samples at t = 0.
(c): 100 μL of 0.1N HCl solution added into the sample after PLM analysis at 6 hrs.

TABLE 7
Physical Stability Assessment at 40° C./75%
RH/7 d Condition for Rifaximin and Rifaximin
Dispersions Prepared in Methanol by Spray Drying

| Description (a) | Habit/Description | Analysis | Results |
|---|---|---|---|
| (100:0) | orange solids, dry | PLM | agg., irr., no B/E |
| Rifaximin-only | | | |
| (25:75) | dark yellow | PLM | agg., irr., no B/E |
| PVP K-90 | solids, dry | | |
| (50:50) | orange solids, dry | PLM | agg., irr., no B/E |
| PVP K-90 | | | |
| (75:25) | orange solids, dry | PLM | agg., irr., no B/E |
| PVP K-90 | | | |
| (25:75) | light orange | PLM | agg., irr., no B/E |
| HPMC-P | solids, dry | mDSC | 148° C. (T_{g}, midpoint); |
| | | | 0.3 J/g · ° C. (ΔCₚ) |
| (50:50) | orange solids, dry | PLM | agg., irr., no B/E |
| HPMC-P | | | |
| (75:25) | orange solids, dry | PLM | agg., irr., no B/E |
| HPMC-P | | | |
| (25:75) | light orange | PLM | agg., irr., no B/E |
| HPMC-AS HG | solids, dry | | |
| (50:50) | orange solids, dry | PLM | agg., irr., no B/E |
| HPMC-AS HG | | | |
| (75:25) | orange solids, dry | PLM | agg., irr., no B/E |
| HPMC-AS HG | | mDSC | 177° C. (T_{g}, midpoint); |
| | | | 0.5 J/g · ° C. (ΔCₚ) |
| (25:75) | light orange | PLM | agg., irr., no B/E |
| HPMC-AS MG | solids, dry | | |
| (50:50) | orange solids, dry | PLM | agg., irr., no B/E |
| HPMC-AS MG | | | |
| (75:25) | orange solids, dry | PLM | agg., irr., no B/E |
| HPMC-AS MG | | mDSC | 152° C. (T_{g}, midpoint) |
| (25:75) | light orange | PLM | agg., irr., no B/E |
| Eudragit | solids, dry | mDSC | 140° C. (T_{g}, midpoint); |
| L100-55 | | | 0.5 J/g · ° C. (ΔCₚ) |
| (50:50) | orange solids, dry | PLM | agg., irr., no B/E |
| Eudragit | | | |
| L100-55 | | | |
| (75:25) | orange solids, dry | PLM | agg., irr., no B/E |
| Eudragit | | | |
| L100-55 | | | |

(a): approximate ratio of Rifaximin to polymer, by weight.
(b): analysis treated as non-cGMP.

TABLE 8
Physical Stability Assessment at 60° C./Dry/7
d Condition for Rifaximin and Rifaximin Dispersions
Prepared in Methanol by Spray Drying

| Description (a) | Habit/Description | Analysis | Results |
|---|---|---|---|
| (100:0) | orange solids | PLM | agg., irr., no B/E |
| Rifaximin-only | | | |
| (25:75) | orange solids | PLM | agg., irr., no B/E |
| PVP K-90 | | | |
| (50:50) | orange solids | PLM | agg., irr., no B/E |
| PVP K-90 | | | |
| (75:25) | orange solids | PLM | agg., irr., no B/E |
| PVP K-90 | | | |
| (25:75) | light orange | PLM | agg., irr., no B/E |
| HPMC-P | solids | | |
| (50:50) | orange solids | PLM | agg., irr., no B/E |
| HPMC-P | | | |
| (75:25) | orange solids | PLM | agg., irr., no B/E |
| HPMC-P | | | |
| (25:75) | light orange | PLM | agg., irr., no B/E |
| HPMC-AS HG | solids | | |
| (50:50) | orange solids | PLM | agg., irr., no B/E |
| HPMC-AS HG | | | |
| (75:25) | orange solids | PLM | agg., irr., no B/E |
| HPMC-AS HG | | | |
| (25:75) | light orange | PLM | agg., irr., no B/E |
| HPMC-AS MG | solids | | |
| (50:50) | orange solids | PLM | agg., irr., no B/E |
| HPMC-AS MG | | | |
| (75:25) | orange solids | PLM | agg., irr., no B/E |
| HPMC-AS MG | | | |
| (25:75) | light orange | PLM | agg., irr., no B/E |
| Eudragit | solids | | |
| L100-55 | | | |
| (50:50) | orange solids | PLM | agg., irr., no B/E |
| Eudragit | | | |
| L100-55 | | | |
| (75:25) | orange solids | PLM | agg., irr., no B/E |
| Eudragit | | | |
| L100-55 | | | |

(a): approximate ratio of Rifaximin to polymer, by weight.

TABLE 9
Parameters for Rifaximin Solid Dispersions by Spray Drying

| | | | | Inlet temp. | Outlet temp. | Spray rate |
|---|---|---|---|---|---|---|
| Description | Inlet temp. | | | (measured, | (measured, | (b) |
| (a) | (set, ° C.) | Aspirator % | Pump % | ° C.) | ° C.) | mL/min |
| (50:50) | 120 | 95 | 40-30 | 120-119 | 60-45 | 9.6 |
| HPMC-AS | | | | | | |
| MG, ~10 g | | | | | | |
| scale | | | | | | |
| (25:75) | 120 | 95 | 45-30 | 120-119 | 55-43 | 9.7 |
| HPMC-P, | | | | | | |
| ~10 g scale | | | | | | |

(a): approximate ratio of Rifaximin to polymer, by weight.
(b): flow rates are estimated at 30% pump.

TABLE 10
Characterizations of 50:50 (w/w) Rifaximin/HPMC-AS
MG Dispersion by Spray Drying

| Analysis | Results |
|---|---|
| XRPD | x-ray amorphous |
| mDSC | 154° C. (midpoint, T_{g}) |
| | 0.4 J/g · ° C. (ΔCₚ) |
| TG-IR | 0.5 wt % |
| | (loss up to 100° C.) |
| | 199° C. |
| | (onset, apparent decomp.) |
| | water, methanol and unknown volatiles |

TABLE 11
Characterizations of 25:75 (w/w) Rifaximin/HPMC-P
Dispersion by Spray Drying

| Analysis | Results |
|---|---|
| XRPD | x-ray amorphous |
| mDSC | 152° C. (midpoint, T_{g}) |
| | 0.4 J/g · ° C. (ΔCp) |
| TG-IR | 1.5 wt % |
| | (loss up to 100° C.) |
| | 178° C. |
| | (onset, apparent decomp.) |
| | water and methanol |

TABLE 12
Sample Information of Rifaximin Dispersions for Dissolution
Test in pH 6.52 FASSIF Buffer at 37° C.

| | Sample | Dissolution | Solids | Volume of |
|---|---|---|---|---|
| Description (a) | ID | Vessel No | Weight (mg) | Buffer (mL) |
| (50:50) | 4042-97- | 1 | 122.1 | 300 |
| HPMC-AS MG | 01 | 2 | 120.5 | |
| | | 3 | 121.4 | |
| (25:75) | 4103-01- | 4 | 242.5 | 300 |
| HPMC-P | 01 | 5 | 239.2 | |
| | | 6 | 242.4 | |

(a): approximate ratio of Rifaximin to polymer, by weight.

TABLE 13
Rifaximin Concentrations of 50:50 (w/w) Rifaximin/HPMC-AS
MG Dispersion in pH 6.52 FASSIF Buffer at 37° C.

| Dissolution | Time | | | Concentration |
|---|---|---|---|---|
| Vessel No | (min.) | Dilution (c) | Absorbance (d) | (μg/mL) |
| 1 | 5 | — | 0.0159 | 0.34 |
| | 10 | — | 0.0346 | 2.53 |
| | 15 | — | 0.0569 | 5.13 |
| | 30 | — | 0.09655 | 9.75 |
| | 60 | — | 0.1626 | 17.46 |
| | 90 | — | 0.2216 | 24.35 |
| | 120 | — | 0.25625 | 28.39 |
| | 1440 | 4 | 0.4093 | 184.99 |
| 2 | 5 | 2 | 0.02895 | 3.73 |
| | 10 | — | 0.0304 | 2.04 |
| | 15 | — | 0.04655 | 3.92 |
| | 30 | — | 0.104 | 10.62 |
| | 60 | — | 0.17755 | 19.21 |
| | 90 | — | 0.248 | 27.43 |
| | 120 | — | 0.3065 | 34.25 |
| | 1440 | 4 | 0.3944 | 178.04 |
| 3 | 5 | — | 0.0107 | −0.26 |
| | 10 | — | 0.02555 | 1.47 |
| | 15 | — | 0.03975 | 3.13 |
| | 30 | — | 0.08735 | 8.68 |
| | 60 | — | 0.1766 | 19.10 |
| | 90 | — | 0.25815 | 28.61 |
| | 120 | — | 0.32055 | 35.89 |
| | 1440 | 4 | 0.4202 | 190.08 |

(c): certain samples were diluted before analyzed to avoid the possibility of falling outside the linearity range of the instrument.
(d): absorbance data less than 0.05 is below instrument detection limit and therefore concentration calculated from such absorbance is an approximate value.

TABLE 14
Rifaximin Concentrations of 25:75 (w/w) Rifaximin/HPMC-P
Dispersion in pH 6.52 FASSIF Buffer at 37° C.

| Dissolution | Time | | | Concentration |
|---|---|---|---|---|
| Vessel No | (min.) | Dilution (d) | Absorbance (e) | (μg/mL) |
| 4 | 5 | — | 0.01555 | 0.30 |
| | 10 | — | 0.03395 | 2.45 |
| | 15 | — | 0.0528 | 4.65 |
| | 30 | — | 0.12235 | 12.77 |
| | 60 | — | 0.2643 | 29.33 |
| | 90 | — | 0.37355 | 42.08 |
| | 120 | — | 0.455 | 51.58 |
| | 1440 | 4 | 0.39465 | 178.16 |
| 5 | 5 | — | 0.0329 | 2.33 |
| | 10 | — | 0.06805 | 6.43 |
| | 15 | — | 0.07905 | 7.71 |
| | 30 | — | 0.13745 | 14.53 |
| | 60 | — | 0.242 | 26.73 |
| | 90 | — | 0.32595 | 36.52 |
| | 120 | — | 0.40555 | 45.81 |
| | 1440 | 4 | 0.38525 | 173.77 |
| 6 | 5 | — | 0.0155 | 0.30 |
| | 10 | — | 0.057 | 5.14 |
| | 15 | — | 0.09415 | 9.47 |
| | 30 | — | 0.17145 | 18.49 |
| | 60 | — | 0.2724 | 30.27 |
| | 90 | — | 0.36815 | 41.45 |
| | 120 | — | 0.43155 | 48.84 |
| | 1440 | 4 | 0.3838 | 173.09 |

(d): certain samples were diluted before analyzed to avoid the possibility of falling outside the linearity range of the instrument.
(e): absorbance data less than 0.05 is below instrument detection limit and therefore concentration calculated from such absorbance is an approximate value.

TABLE 15
Averaged Concentrations of 50:50 (w/w) Rifaximin/HPMC-AS
MG Dispersions in pH 6.52 FASSIF Buffer at 37° C.

| | | | | Average | |
|---|---|---|---|---|---|
| | | | Concen- | Concen- | |
| | Dissolution | Time | tration | tration | Standard |
| Description (a) | Vessel No | (min.) | (μg/mL) | (μg/mL) | Deviation |
| (50:50) | 1 | 5 | 0.34 | 1.27^{b} | 2.154 |
| HPMC-AS MG | 2 | | 3.73 | | |
| | 3 | | −0.26 | | |
| | 1 | 10 | 2.53 | 2.01^{b} | 0.5284 |
| | 2 | | 2.04 | | |
| | 3 | | 1.47 | | |
| | 1 | 15 | 5.13 | 4.06^{b} | 1.008 |
| | 2 | | 3.92 | | |
| | 3 | | 3.13 | | |
| | 1 | 30 | 9.75 | 9.69 | 0.970 |
| | 2 | | 10.62 | | |
| | 3 | | 8.68 | | |
| | 1 | 60 | 17.46 | 18.59 | 0.977 |
| | 2 | | 19.21 | | |
| | 3 | | 19.10 | | |
| | 1 | 90 | 24.35 | 26.80 | 2.202 |
| | 2 | | 27.43 | | |
| | 3 | | 28.61 | | |
| | 1 | 120 | 28.39 | 32.85 | 3.945 |
| | 2 | | 34.25 | | |
| | 3 | | 35.89 | | |
| | 1 | 1440 | 184.99 | 184.37 | 6.0455 |
| | 2 | | 178.04 | | |
| | 3 | | 190.08 | | |

(a): approximate ratio of Rifaximin to polymer, by weight.
^{b}absorbance data less than 0.05 is below instrument detection limit and therefore concentration calculated from such absorbance is an approximate value.

TABLE 16
Averaged Concentrations of 25:75 (w/w) Rifaximin/HPMC-P
Dispersions in pH 6.52 FASSIF Buffer at 37° C.

| | | | | Average | |
|---|---|---|---|---|---|
| | | | Concen- | Concen- | |
| | Dissolution | Time | tration | tration | Standard |
| Description (a) | Vessel No | (min.) | (μg/mL) | (μg/mL) | Deviation |
| (25:75) | 4 | 5 | 0.30 | 0.98^{b} | 1.171 |
| HPMC-P | 5 | | 2.33 | | |
| | 6 | | 0.30 | | |
| | 4 | 10 | 2.45 | 4.67^{b} | 2.030 |
| | 5 | | 6.43 | | |
| | 6 | | 5.14 | | |
| | 4 | 15 | 4.65 | 7.28 | 2.442 |
| | 5 | | 7.71 | | |
| | 6 | | 9.47 | | |
| | 4 | 30 | 12.77 | 15.26 | 2.935 |
| | 5 | | 14.53 | | |
| | 6 | | 18.49 | | |
| | 4 | 60 | 29.33 | 28.78 | 1.840 |
| | 5 | | 26.73 | | |
| | 6 | | 30.27 | | |
| | 4 | 90 | 42.08 | 40.02 | 3.041 |
| | 5 | | 36.52 | | |
| | 6 | | 41.45 | | |
| | 4 | 120 | 51.58 | 48.75 | 2.886 |
| | 5 | | 45.81 | | |
| | 6 | | 48.84 | | |
| | 4 | 1440 | 178.16 | 175.01 | 2.749 |
| | 5 | | 173.77 | | |
| | 6 | | 173.09 | | |

(a): approximate ratio of Rifaximin to polymer, by weight.
^{b}absorbance data less than 0.05 is below instrument detection limit and therefore concentration calculated from such absorbance is an approximate value.

TABLE 17
Analysis of Rifaximin Dispersions after Dissolution
Test in pH 6.52 FASSIF Buffer at 37° C.

| | Dissolution | | |
|---|---|---|---|
| Description (a) | Vessel No | Analysis | Results |
| (50:50) | 1 | PLM | no B/E observed |
| HPMC-AS | | | change view field, no B/E |
| MG | 2 | PLM | no B/E observed |
| | | | change view field, no B/E |
| | 3 | PLM | no B/E observed |
| | | | majority no B/E, only 1 B/E |
| | | | particle in view field |
| (25:75) | 4 | PLM | B/E flakes and blades |
| HPMC-P | 5 | PLM | no B/E material + B/E flakes |
| | 6 | PLM | no B/E material + B/E flakes |
| | | | & blades |

(a): approximate ratio of Rifaximin to polymer, by weight.

### Abbreviations

| Type | Abbreviation | Full Name/Description |
|---|---|---|
| INSTRU- | XRPD | x-ray powder diffractometry |
| MENTAL | mDSC | modulated differential scanning |
| | | calorimetry |
| | TG-IR | thermogravimetric infrared |
| | PLM | polarized light microscopy |
| | UV | ultraviolet spectroscopy |
| POLYMER | HPMC-AS | hydroxypropylmethyl cellulose acetate |
| | | succinate |
| | HPMC-P | hydroxypropylmethyl cellulose phthalate |
| | Eudragit | anionic polymers with methacrylic acid |
| | L100 | as a functional group, dissolution at |
| | | pH > 6.0 |
| | PVP K-90 | polyvinylpyrrolidone, grade K-90 |
| RESULTS | T_{g} | glass transition temperature |
| | ΔCₚ | heat of capacity change |
| | amorph. | amorphous |
| | agg. | aggregates |
| | irr. | irregular |
| | decomp. | decomposition |
| | B | birefringence |
| | E | extinction |

### Example 2. Ternary Dispersion of 50:50 (w/w) Rifaximin:HPMC-AS MG

A ternary dispersion of 50:50 (w/w) Rifaximin:HPMC-AS MG with 5.9 wt % Pluronic F-127 was prepared in large quantity (containing approximately 110 g of Rifaximin) by spray drying. Disclosed herein are the analytical characterizations for Rifaximin ternary dispersion as-prepared and post-stress samples at 70° C./75% RH for 1 week and 3 week, and post-stress sample at 40° C./75% RH for 6 weeks and 12 weeks.

### Characterization of Rifaximin Ternary Dispersion

Characterizations of the spray dried Rifaximin ternary dispersion (50:50 (w/w) rifaximin:HPMC-AS MG with 5.9 wt % Pluronic F-127) are described in Table 18.

TABLE 18
Characterizations of Combined Rifaximin
Ternary Dispersion Solids - Spray Drying

| Sample ID | Analysis | Results (b) |
|---|---|---|
| 4103-74-01a | XRPD | x-ray amorphous |
| | mDSC | 136° C. (midpoint, T_{g}) |
| | | 0.4 J/g · ° C. (ΔCp) |
| | TG-IR | 0.7 wt % |
| | | (loss up to 100° C.) |
| | | 202° C. |
| | | (onset, volatilization and apparent decomp.) |
| | | methanol and possible acetic acid |
| | IR-ATR | consistent with structure |
| | Raman | consistent with structure |
| | SEM | agglomerates of collapsed spheres |
| | PLM | irregularly-shaped equant particles |
| | PSA | d10 (μm): 3.627, d50 (μm): 8.233, |
| | | d90 (μm): 17.530 |
| | DVS | 0.13 wt % (loss at 5% RH) |
| | | 11.14 wt % (gain, 5-95% RH) |
| | | 10.80 wt % (loss, 95-5% RH) |
| 4074-89-01 (c) | XRPD | x-ray amorphous |

(b): temperatures are round to the nearest degree; ΔCp is rounded to one decimal places and wt % is rounded to one decimal place.

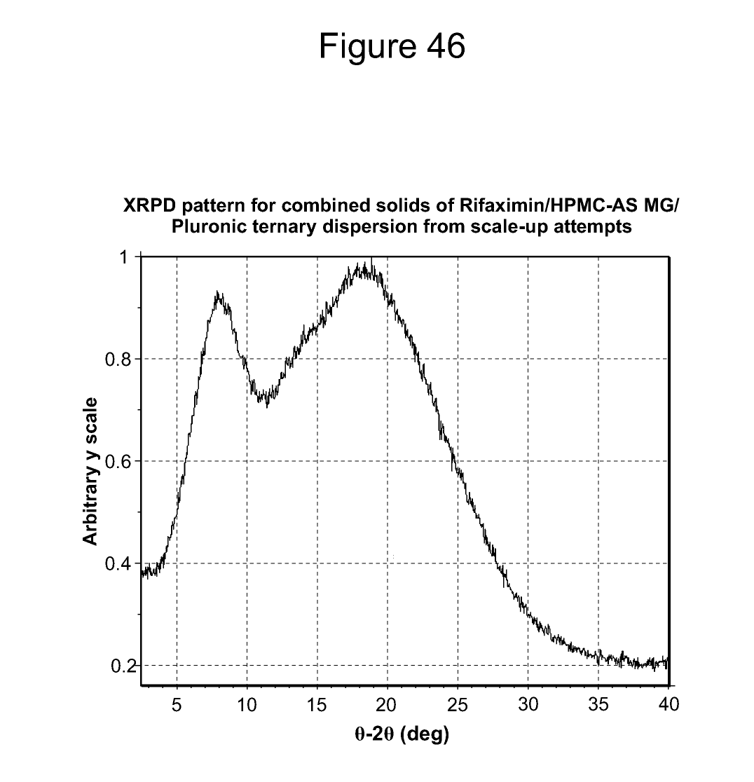

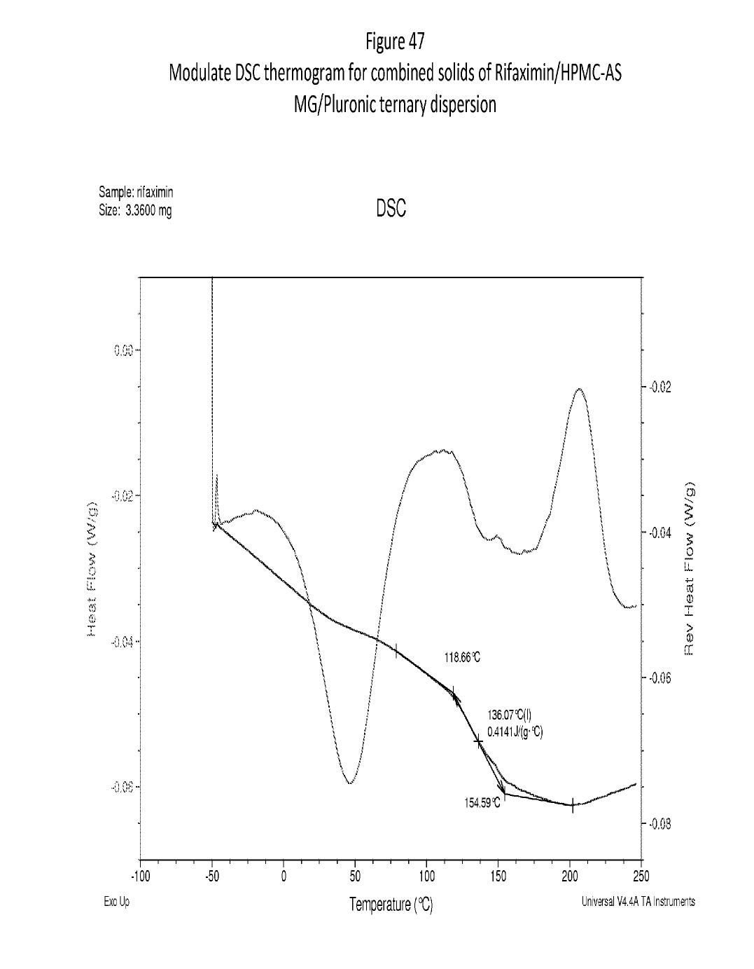

A high resolution XRPD pattern was acquired and material is x-ray amorphous (FIG. 46). By mDSC (FIG. 47), a single apparent T_{g} is observed from the step change in the reversing heat flow signal at approximately 136° C. with a heat capacity change at T_{g} of approximately 0.4 J/g·° C.

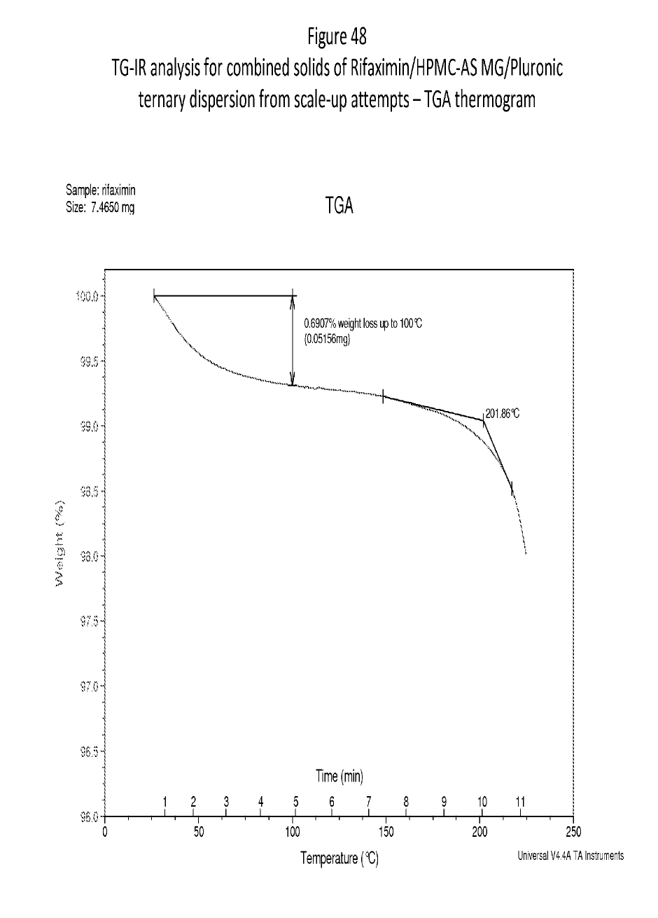

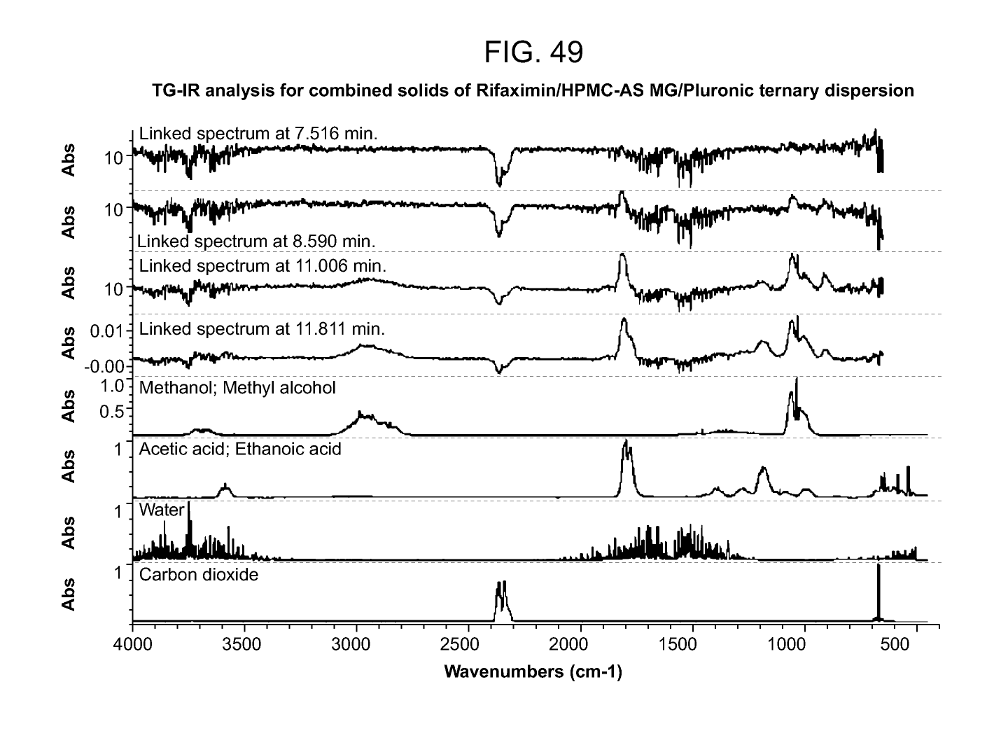

Thermogravimetric analysis coupled with infra-red spectroscopy (TG-IR) was performed to analyze volatiles generated upon heating. The total weight loss of sample was approximately 0.7 wt % to 100° C. and the dramatic change in the slope occurs at approximately 202° C. (FIG. 48). The Gram-Schmidt plot corresponds to the overall IR intensity associated with volatiles released by a sample upon heating at 20° C./min. By Gram-Schmidt, a negligible increase of intensity upon heating is observed before ˜7 minutes followed by a dramatic increase of intensity with the maximum at ˜11.8 min. The waterfall plot (data not shown) of this sample indicates volatile are released upon heating after ˜7 min (data is shown in FIG. 49 using the linked IR spectrum at different time points as an example) and volatiles were identified as residual methanol from the processing solvent in spray drying and possible acetic acid from HPMC-AS MG.

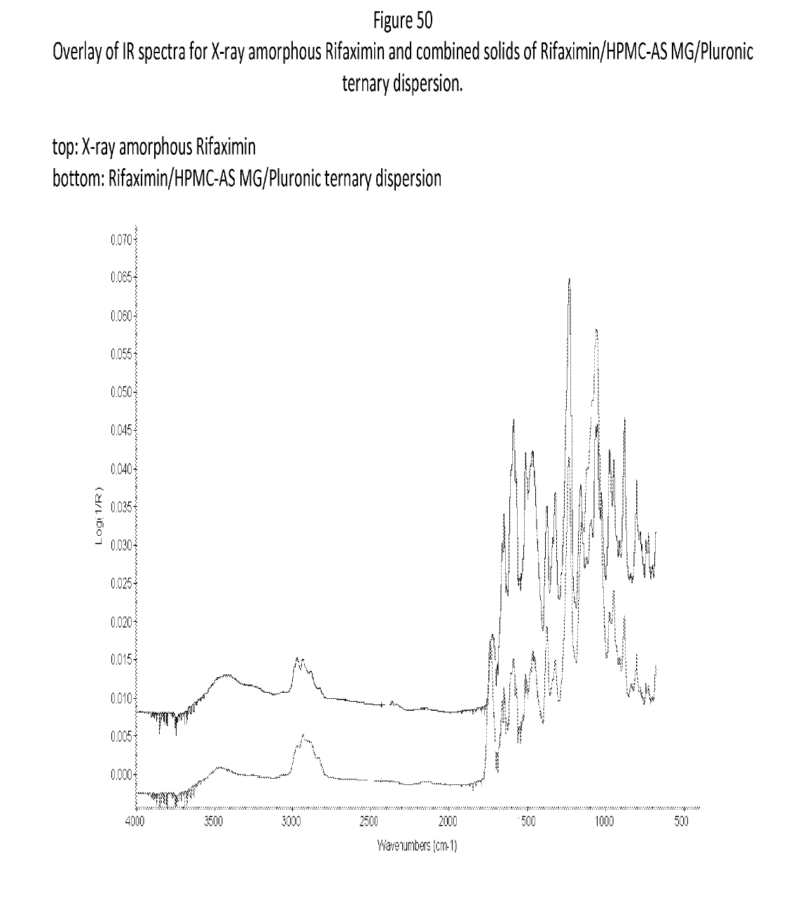

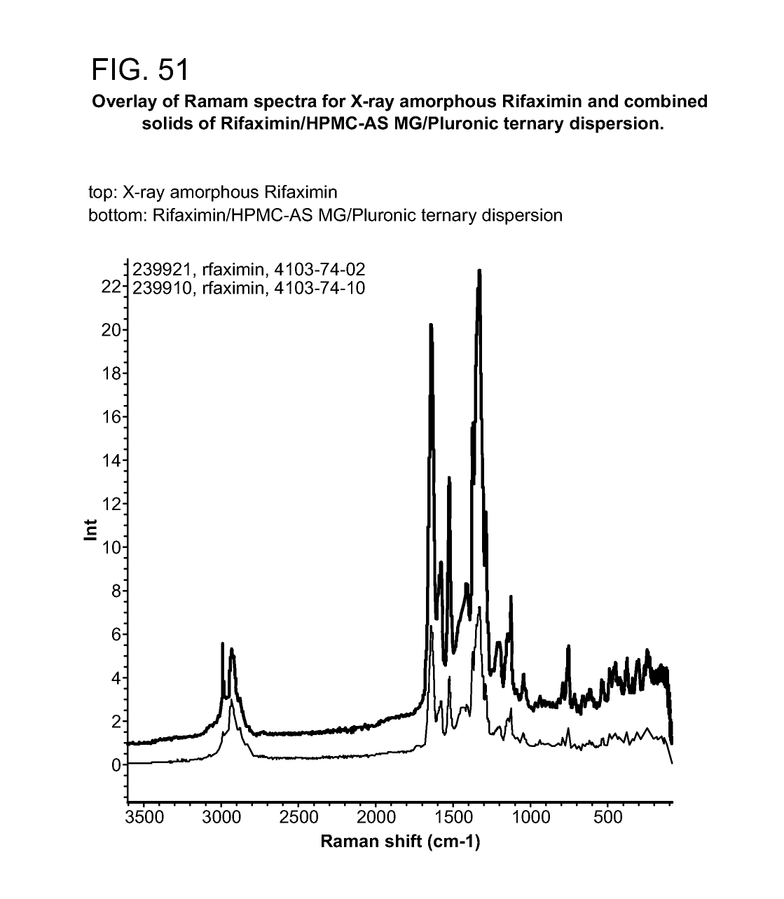

Vibrational spectroscopy techniques, including IR and Raman were employed to further characterize this ternary dispersion. The overlay of IR spectra for the dispersion and X-ray amorphous Rifaximin is shown in FIG. 50. Based on visual inspection, two spectra are very similar. Similar observations can be drawn from the comparison of Raman analysis (FIG. 51). The sample is composed of agglomerates of collapsed spheres. Particles sizes of spheres are not uniform, ranging from slightly larger to much less than 10 μm.

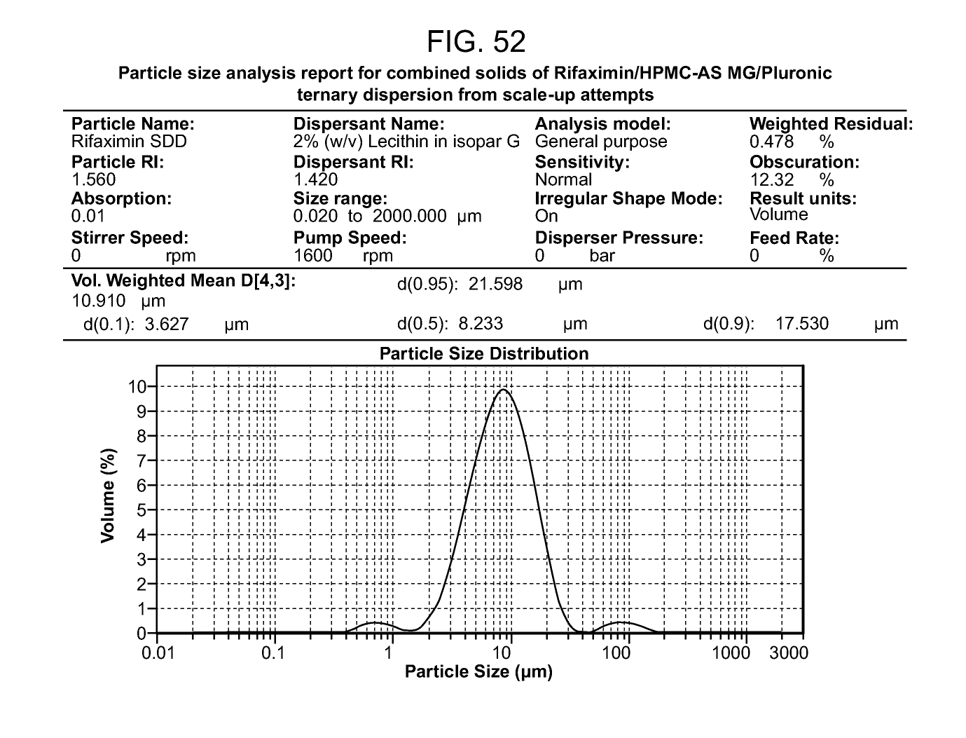

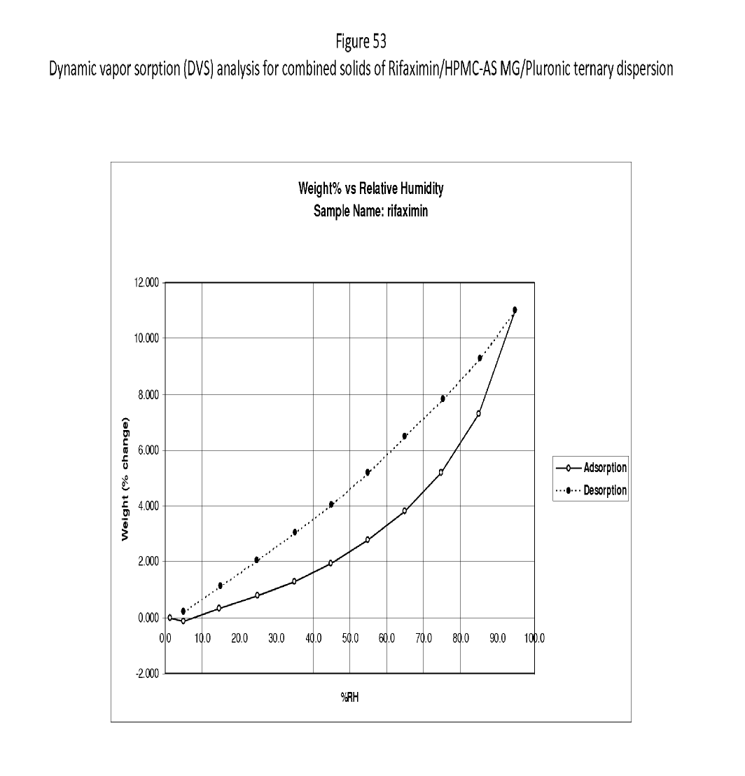

PLM images (data not shown) of solids dispersed in mineral oil were collected, which indicate sample primarily is composed of irregularly-shaped equant particles approximately 5-15 μm in length with some agglomerates 20-50 μm in length. Particle size analysis (FIG. 52) indicates that 50% of particles have size less than 8.233 μm and 90% of particles have size less than 17.530 μm. Data was acquired in 2% (w/v) Lecithin in Isopar G.

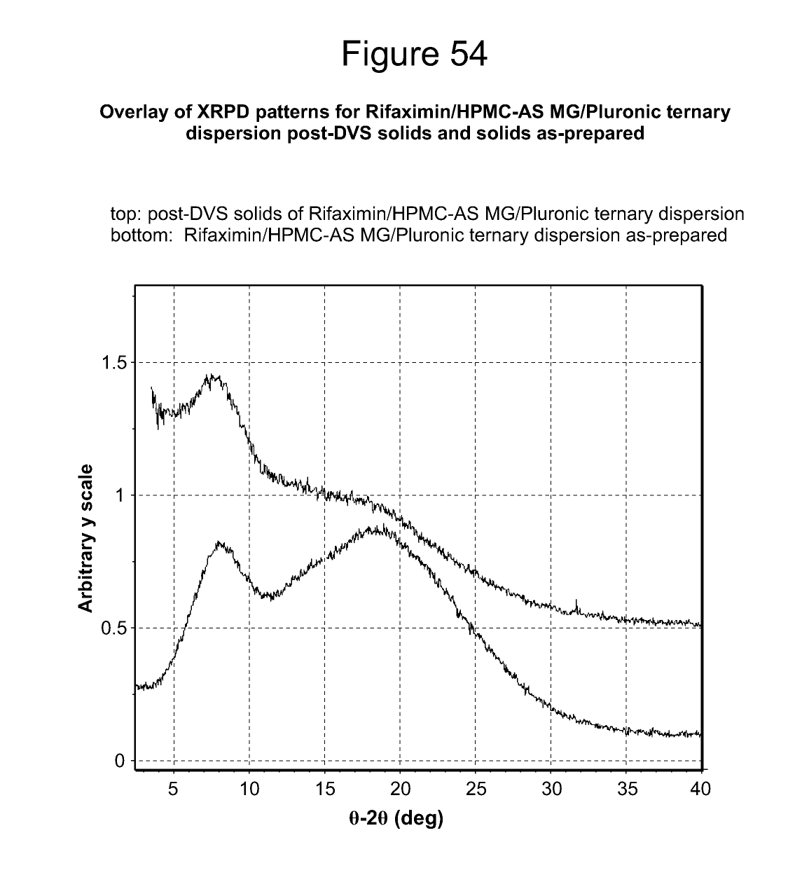

The DVS isotherm of solids is shown in FIG. 53. The material exhibits a 0.13 wt % loss upon equilibration at 5% RH. Solids then gain 11.14 wt % between 5% and 95% RH and exhibits some hysteresis with 10.80 wt % loss upon desorption from 95% to 5% RH. XRPD analysis of the solids recovered after completion of the desorption step showed no evidence of sharp peaks indicative of a crystalline solid (FIG. 54).

### Physical Stability Assessment on Rifaximin Ternary Dispersion

An assessment of physical stability of this rifaximin ternary dispersion is currently in progress by exposing solids to varied elevated temperature/relative humidity conditions, including 25° C./60% RH, 40° C./75% RH and 70° C./75% RH for extended period of time. At designated time interval, such as at 1 week, 3 week, 6 week, and 12 weeks, selected samples were removed from stress conditions for characterization.

Table 19 summarized characterization results for the samples that stressed at 70° C./75% RH condition 1 week and 3 weeks, and the sample that stressed at 40° C./75% RH condition 6 weeks.

TABLE 19
Physical Stability Evaluation on Rifaximin Ternary Dispersion

| | | Habit/ | | |
|---|---|---|---|---|
| Condition | Time | Description | Analysis | Results (a) |
| 70° C./ | 1 week | orange solids, | XRPD | x-ray amorphous |
| 75% RH | | aggregates, | mDSC | 134° C. |
| | | no B/E | | (midpoint, T_{g}) |
| | | | | 0.4 J/g · ° C. (ΔCp) |
| | | | SEM | agglomerates of |
| | | | | collapsed spheres |
| | | | KF | 3.80% |
| 70° C./ | 3 weeks | dark orange | XRPD | x-ray amorphous |
| 75% RH | | solids, | mDSC | 134° C. |
| | | aggregates, | | (midpoint, T_{g}) |
| | | no B/E | | 0.4 J/g · ° C. (ΔCp) |
| | | | SEM | agglomerates of |
| | | | | collapsed spheres |
| | | | KF | 3.19% |
| 40° C./ | 6 weeks | orange solids, | XRPD | x-ray amorphous |
| 75% RH | | aggregates, | mDSC | 133° C. |
| | | no B/E | | (midpoint, T_{g}) |
| | | | | 0.4 J/g · ° C. (ΔCp) |
| | | | SEM | agglomerates of |
| | | | | collapsed spheres |
| | | | KF | 4.05% |
| 40° C./ | 12 weeks | orange solids, | XRPD | x-ray amorphous |
| 75% RH | | aggregates, | mDSC | 132° C. |
| | | no B/E | | (midpoint, T_{g}) |
| | | | | 0.5 J/g · ° C. (ΔCp) |
| | | | SEM | agglomerates of |
| | | | | collapsed spheres |
| | | | KF | 3.37% |

(a): temperatures are round to the nearest degree; ΔCp is rounded to one decimal places.

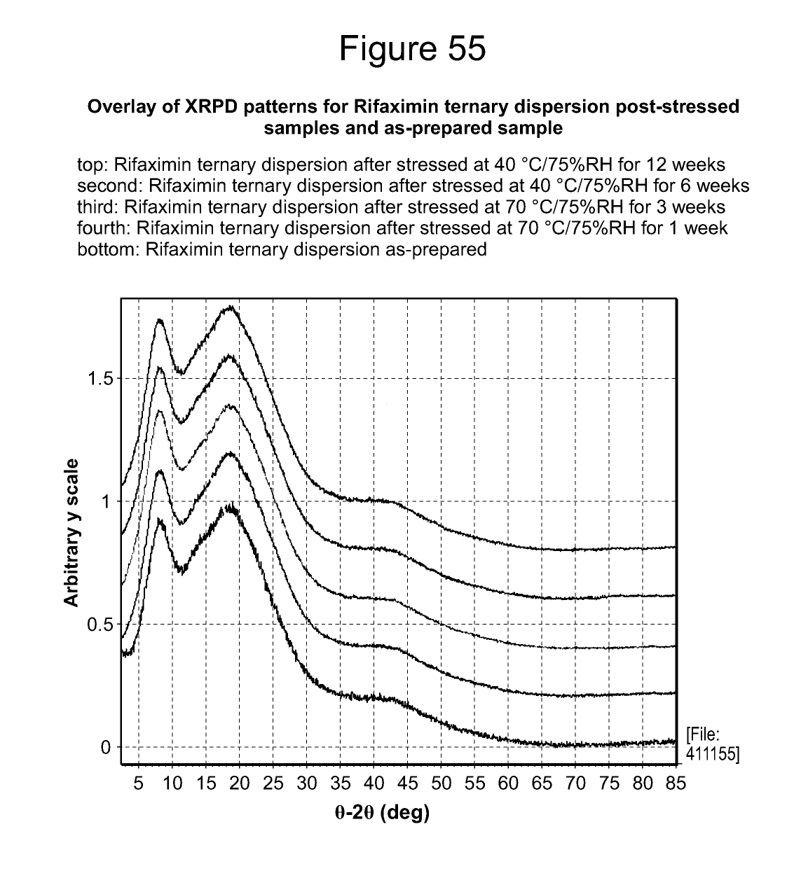

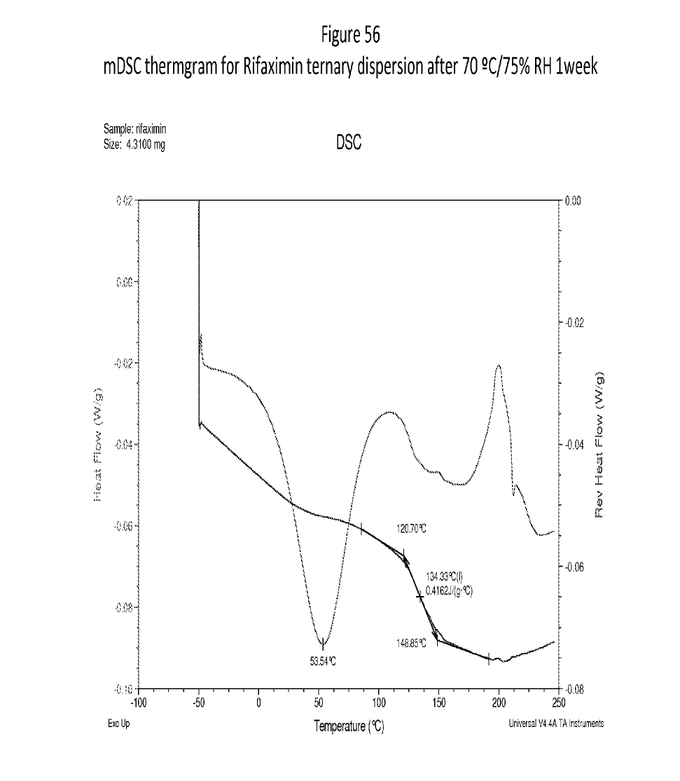

For a sample that was stressed at 70° C./75% RH for 1 week, solids are still x-ray amorphous according to XRPD (FIG. 55). A single T_{g} at approximately 134° C. was observed from the apparent step change in the reversing heat flow signal in mDSC with the change of heat capacity 0.4 J/g ° C., indicating the components of each dispersion remained intimately miscible after stress (FIG. 56). A non-reversible endotherm was observed at approximately 54° C. which is likely due to the residual solvent from spray drying and moisture that materials absorbed during stress, which is confirmed by KF analysis that sample contains 3.80 wt % of water (KF analysis for Rifaximin ternary dispersion after 70° C./75% RH 1 week; 1.2855 g−R1=3.72 and 0.988 g−R1=3.87%). The sample is composed of agglomerates of collapsed spheres and particles sizes of spheres are not uniform, which is similar to the as-prepared material.

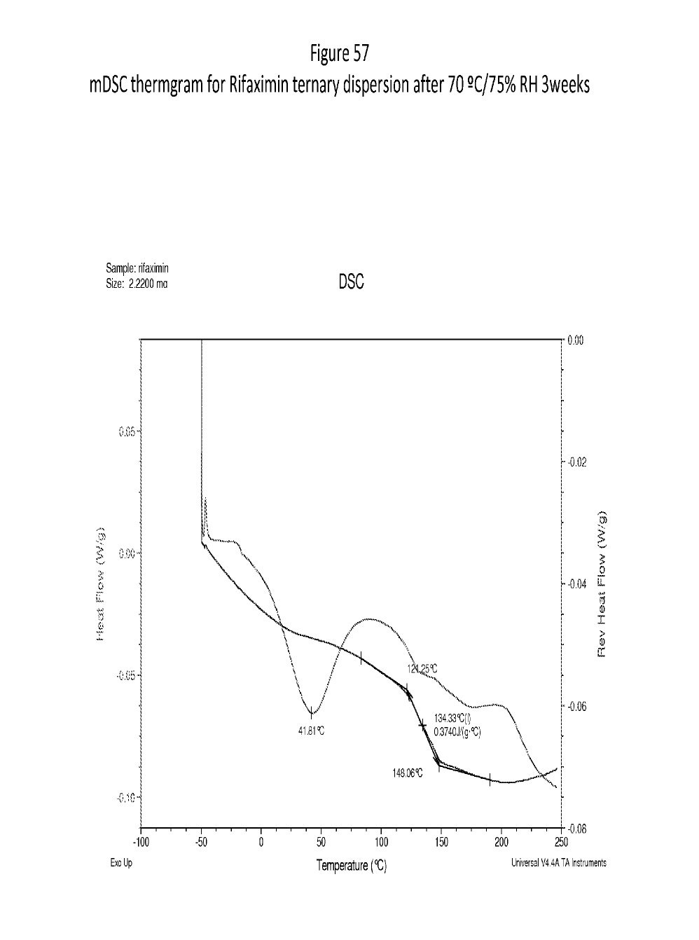

For the sample that was stressed at 70° C./75% RH for 3 weeks, although the color of the material appeared to be darker than the 1-week sample, characterization results for 3-week sample are similar to that for 1-week sample. Solids are also x-ray amorphous by XRPD (FIG. 55) and display a single T_{g} at approximately 134° C. by mDSC (FIG. 57). KF analysis indicates it contains 3.19 wt % of water (KF analysis for rifaximin ternary dispersion after 70° C./75% RH 3 weeks; 1.2254 g−R1=3.45 and 1.1313 g −R1=2.93). By SEM (data not shown), the material has morphology similar to the as-prepared dispersion and 1-week stress sample, which is composed of agglomerates of collapsed spheres and particles sizes of spheres are not uniform.

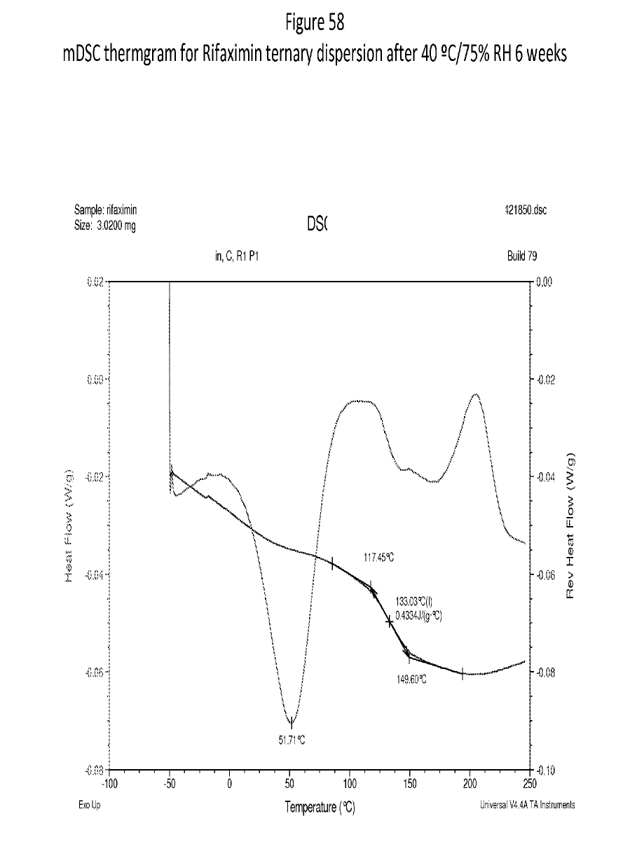

For the sample that was stressed at 40° C./75% RH for 6 weeks, solids are still x-ray amorphous according to XRPD (FIG. 55). It has a single T_{g} at approximately 133° C. by mDSC with the change of heat capacity 0.4 J/g ° C. (FIG. 58). It contains 4.05 wt % of water by KF (KF analysis for rifaximin ternary dispersion after 40° C./75% RH 6 weeks; 1.0947 g−R1=3.47 and 1.2030−R1=4.63). By SEM (data not shown), the sample is composed of agglomerates of collapsed spheres and particles sizes of spheres are not uniform, which is similar to the as-prepared material.

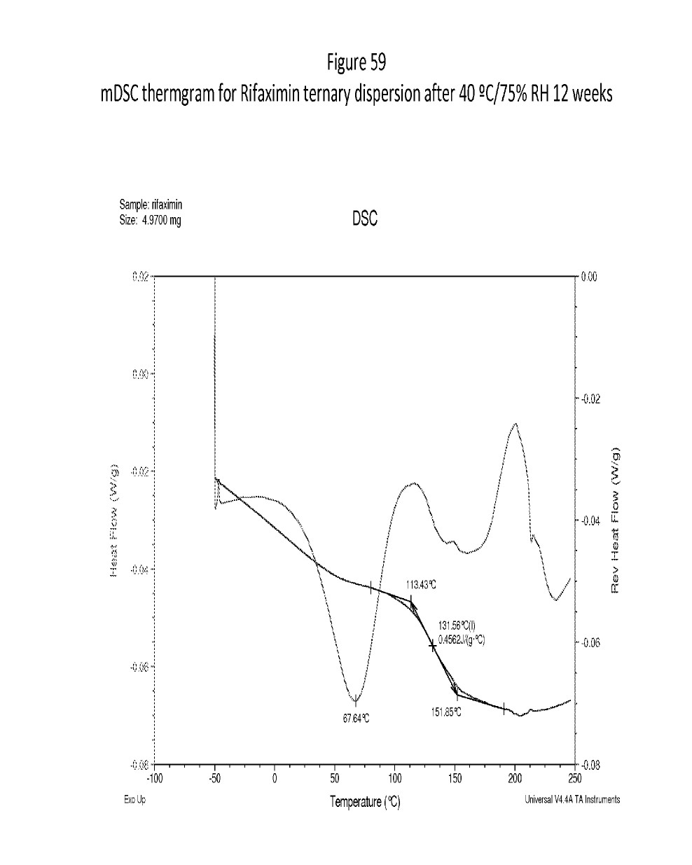

For the sample that was stressed at 40° C./75% RH for 12 weeks, solids are x-ray amorphous (FIG. 55) and display a single T_{g} at approximately 132° C. with the change of heat capacity 0.5 J/g ° C. (FIG. 59). It contains 3.37 wt % of water by KF (KF analysis for Rifaximin ternary dispersion after 40° C./75% RH 12 weeks; 1.3687 g−R1=3.06 and 1.1630 g−R1=3.67). SEM analysis (data not shown) indicates that the sample is composed of agglomerates of collapsed spheres and particles sizes of spheres are not uniform, which is similar to the as-prepared material.

### Example 3. Rifaximin Solid Dispersion Composition and Procedures

### Rifaximin Ternary Dispersion Ingredients

Rifaximin ternary dispersions (50:50 w/w Rifaximin:HPMC-AS MG with 5.9 wt % Pluronic F-127) were prepared from methanol using spray drying in closed mode suitable for processing organic solvents. Ingredients are listed as below in Table 20:

TABLE 20
Components of Rifaximin Solid Dispersion

| Component | mg/g | Purpose |
|---|---|---|
| Rifaximin | 472 | active pharmaceutical ingredient |
| Hydroxypropylmethyl cellulose | 472 | stabilizing agent |
| acetate succinate (HPMC-AS), | | |
| Type MG | | |
| Pluronic F-127 | 56 | wetting agent |
| Methanol | — | volatile; removed during process |

Spray Drying Procedures:

Rifaximin ternary dispersions were prepared by spray drying in both small scale (˜1 g API) and large scale (>34 g API in a single batch).

For the small-scale sample, rifaximin and then the methanol were added to a flask. The mixture was stirred at ambient temperature for ˜5 min to give a clear solution. HPMC-AS MG and Pluronic F-127 were added in succession and the sample was stirred for ˜1 hr. An orange solution was obtained.

For large-scale samples, a solution was prepared at ˜40° C. Rifaximin and then methanol were added to a flask and the mixture was stirred at ˜40° C. for ˜5 min until clear. HPMC-AS MG, and then Pluronic F-127 were added into the rifaximin solution under stirring at ˜40° C. The sample continued to stir for ˜1.5 hr to 2 hr at this temperature. A dark red solution was obtained. The sample was removed from the hot plate and left at ambient to cool.

Experimental conditions to prepare Rifaximin ternary solutions are summarized in Table 21 below:

TABLE 21
Experimental Conditions to Prepare Rifaximin Ternary Solutions

| | weight | | |
|---|---|---|---|
| | (API/HPMC AS MG/ | Temper- | Concentration |
| Solvent | Pluronic F127, g) | ature | (g/L) |
| methanol, 100 mL | 1.0535/1.0529/0.1249 | ambient | 22.3 |
| methanol, 1000 mL | 34.07/34.07/4.02 | ~40° C. | 72.2 |
| methanol, 1250 mL | 50.34/50.32/5.94 | ~40° C. | 85.3 |
| methanol, 1250 mL | 50.16/50.14/5.92 | ~40° C. | 85 |
| methanol, 1250 mL | 50.05/50.06/5.91 | ~40° C. | 85 |

During the spray drying process, both the small and large scale rifaximin ternary solutions were kept at ambient temperature. The pump % was decreased during the process in an attempt to control outlet temperature above 40° C. The operating parameters used for processing are presented in Table 22 below.

TABLE 22
Operating Parameters Used For Processing Rifaximin SD

| | Inlet | | | | | Spray rate |
|---|---|---|---|---|---|---|
| Description | temp. | | | Inlet temp. | Outlet temp. | (b) |
| (a) | (set, ° C.) | Aspirator % | Pump % | (measured, ° C.) | (measured, ° C.) | mL/min |
| 50:50 | 120 | 95 | 35 | 120 | 60-55 | 10.4 |
| Rifaximin:HPMC- | 120 | 95 | 65-30 | 120-119 | 61-42 | 23 |
| AS MG | 120 | 95 | 50-30 | 120-119 | 67-43 | 16 |
| 5.9 wt % | 120 | 95 | 50-30 | 120-119 | 65-43 | 16 |
| Pluronic F-127 | 120 | 95 | 50-30 | 120-119 | 67-43 | 16 |

(a): 50:50 is approximate ratio of Rifaximin to polymer, by weight; 5.9 wt % Pluronic is weight fraction to 50:50 rifaximin:HPMC-AS MG dispersion.
(b): Flow rates are estimated. Flow rate for 4103-41-01 was measured at pump 35%; for 4103-56-01 was measured at pump 65%, while for others were measured at pump 50%.

Solids recovered after spray drying were dried at 40° C. under vacuum for 24 hours and then stored at sub-ambient temperatures over desiccant.

### Spray Drying Process Parameters:

Spray Dryer—PSD 1
Two Fluid Niro Nozzle
Nozzle orifice—1 mm
Inlet gas temperature—125±5 deg C.
Process gas flow (mmH2O)—44
Atomizing gas pressure—0.7-1 bar
Feed rate—4.7 kg/Hr
Outlet temperature—55±3 deg C.
Solution temperature—36 deg C.
Post spray drying vacuum dry at 40 deg C. for 48 hrs

### Example 4

Exemplary formulations for micronized, API, amorphous, solid dispersion and micronized capsules are below in Table 23. These capsules were used in the dog study of Example 5.

TABLE 23
Capsule Formulation composition (Solid Dispersion (SD) Capsules)

| | Micronized | | API | | Amorphous | | SD | | Micronized | |
|---|---|---|---|---|---|---|---|---|---|---|
| | Capsules | | Capsules | | Capsules | | Capsules | | Tablets | |
| Ingredients | % | g/dose | % | g/dose | % | g/dose | % | g/dose | % | g/dose |
| Rifaximin | 95.5 | 2.2 | 47.2 | 2.2 | 51.7 | 2.2 | 42.47 | 2.2 | 50 | 2.2 |
| Ac-di-sol | 4.5 | 0.1 | 5 | 0.23 | 5 | 0.21 | 10.02 | 0.52 | 7.5 | 0.33 |
| Mannitol 160C | | | 47.8 | 2.23 | 43.3 | 1.84 | | | | |
| Pluronic 188 | | | | | | | 5.04 | 0.26 | | |
| HPMC AS | | | | | | | 42.47 | 2.2 | | |
| Avicel 113 | | | | | | | | | 26 | 1.14 |
| Avicel 112 | | | | | | | | | 15 | 0.66 |
| Magnesium | | | | | | | | | 1 | 0.04 |
| Stearate | | | | | | | | | | |
| Cab-o-sil | | | | | | | | | 0.5 | 0.02 |
| Avicel CL-611 | | | | | | | | | | |
| Mannitol 160C | | | | | | | | | | |
| Total | 100 | 2.3 | 100 | 4.66 | 100 | 4.26 | 100 | 5.18 | 100 | 4.4 |

TABLE 24
Manufacture of rifaximin/HPMC-AS/Pluronic 275 mg Capsules

| | % | | Theo. | Actual |
|---|---|---|---|---|
| Component | Formula | mg/caps | Qty (g) | Qty (g) |
| Rifaximin | 42.47 | 275 | 113.7 | 113.7 |
| HPMC-AS | 42.47 | 275 | 113.7 | 113.7 |
| (type MG) | | | | |
| Pluronic F-127 | 5.04 | 32.63 | 13.49 | 13.49 |
| Sodium Croscarmellose | 10.02 | 64.87 | 26.82 | 26.82 |
| Hard Gelatin Capsule | 1 | N/A | 300 | 300 |
| (size 000) Clear | | | | |
| Total | 100 | 647.5 | 267.7 g | |

### Blending/Encapsulation Procedure

To form the capsules sodium croscarmellose was added to the bag of SD rifaximin dispersion and bag blend for 1 minute, and then the material was added to the V-blender and blended for 10 minutes at 24 rpm.

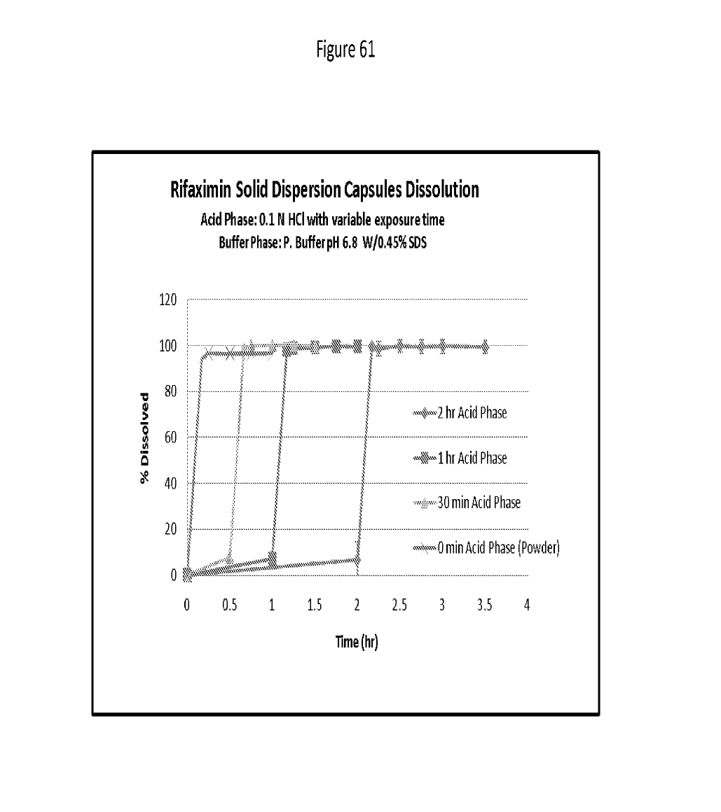

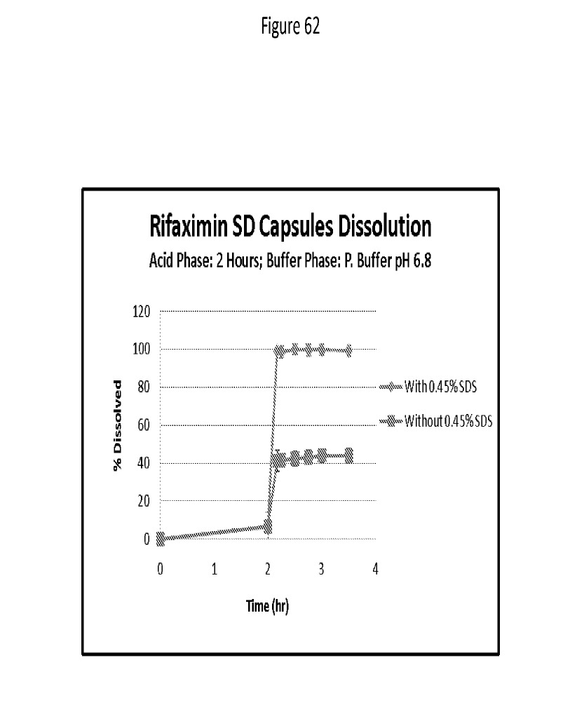

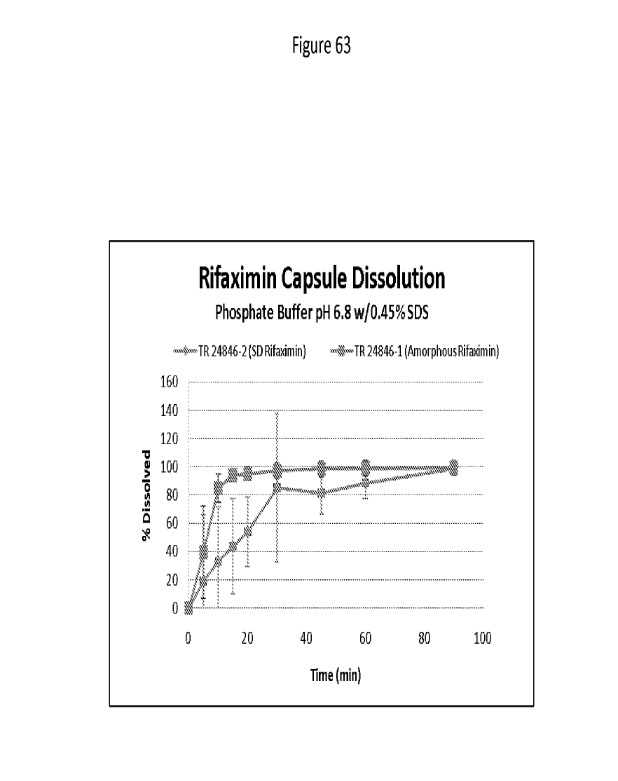

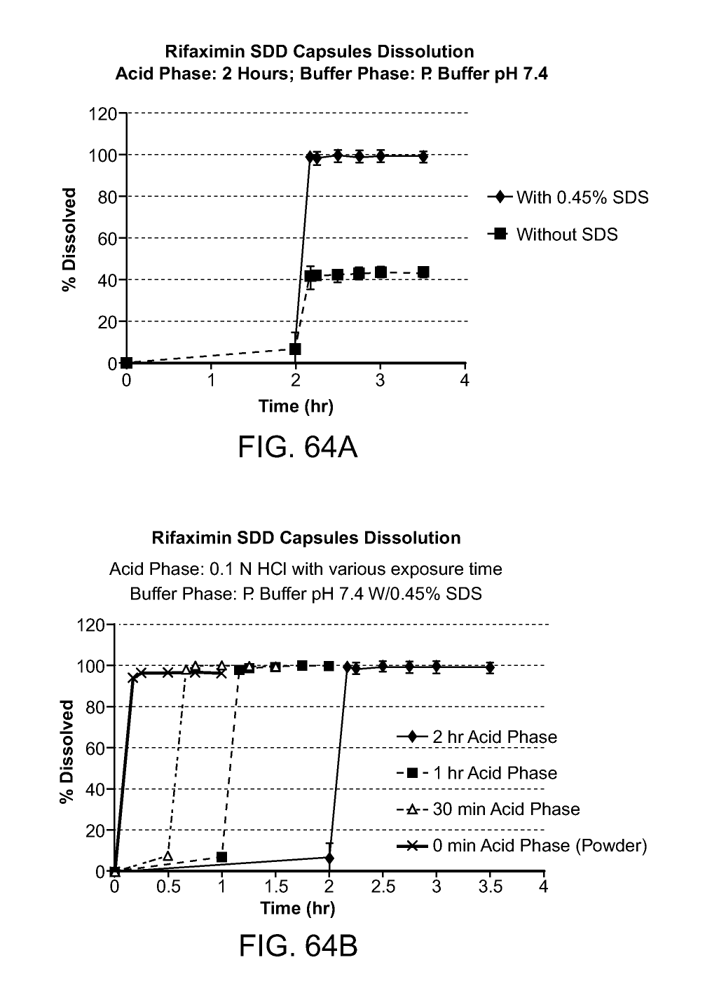

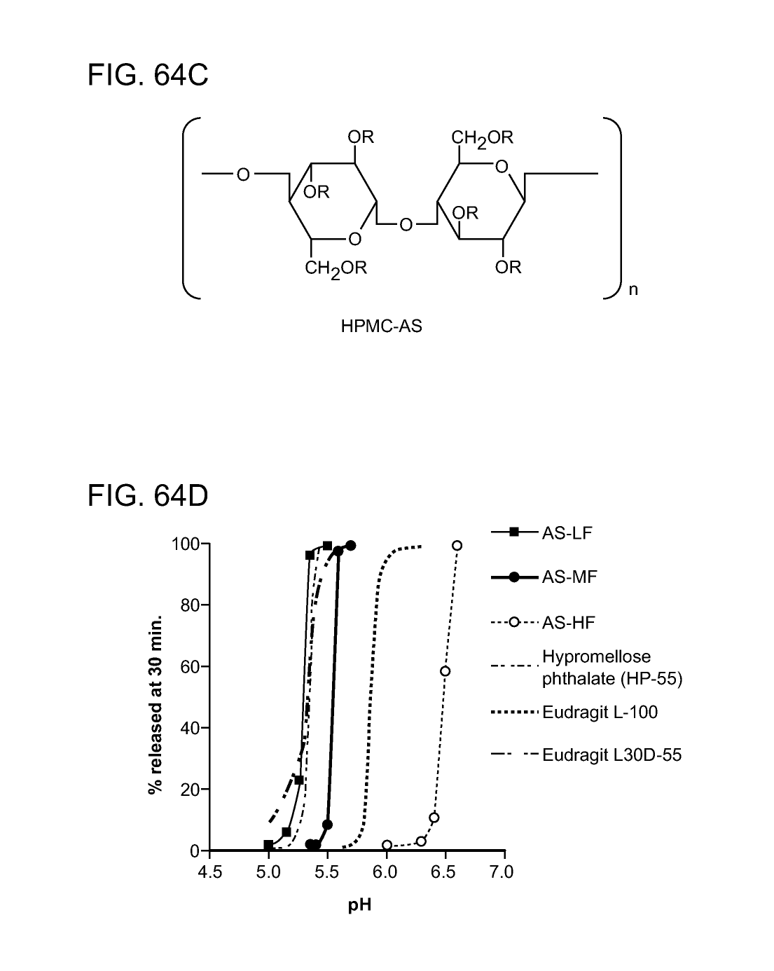

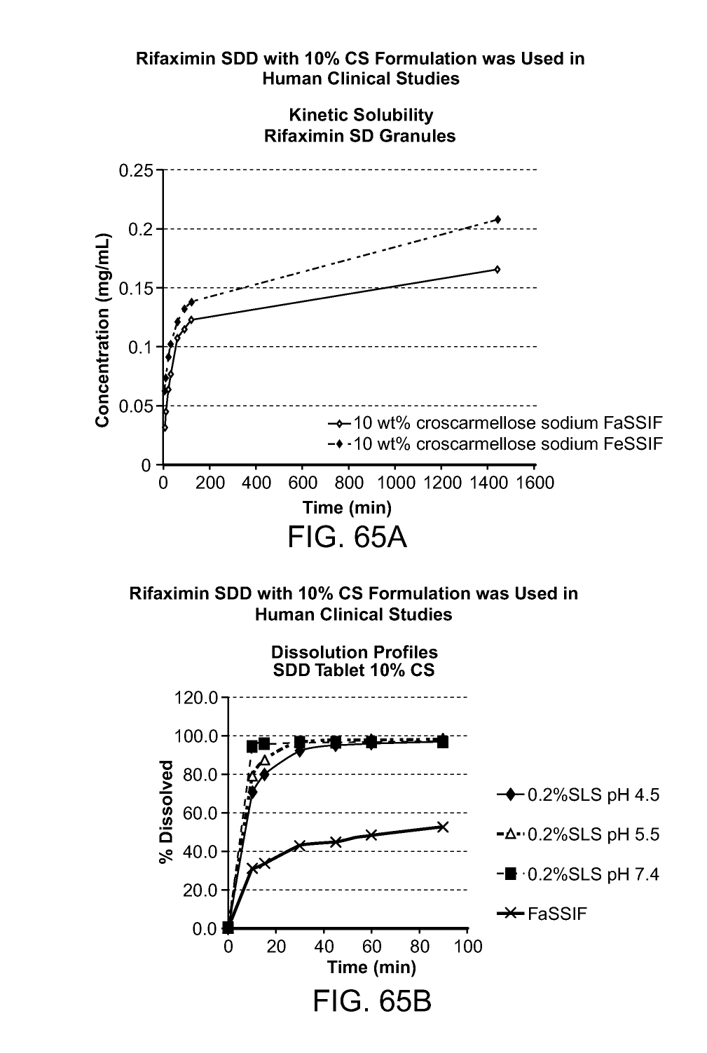

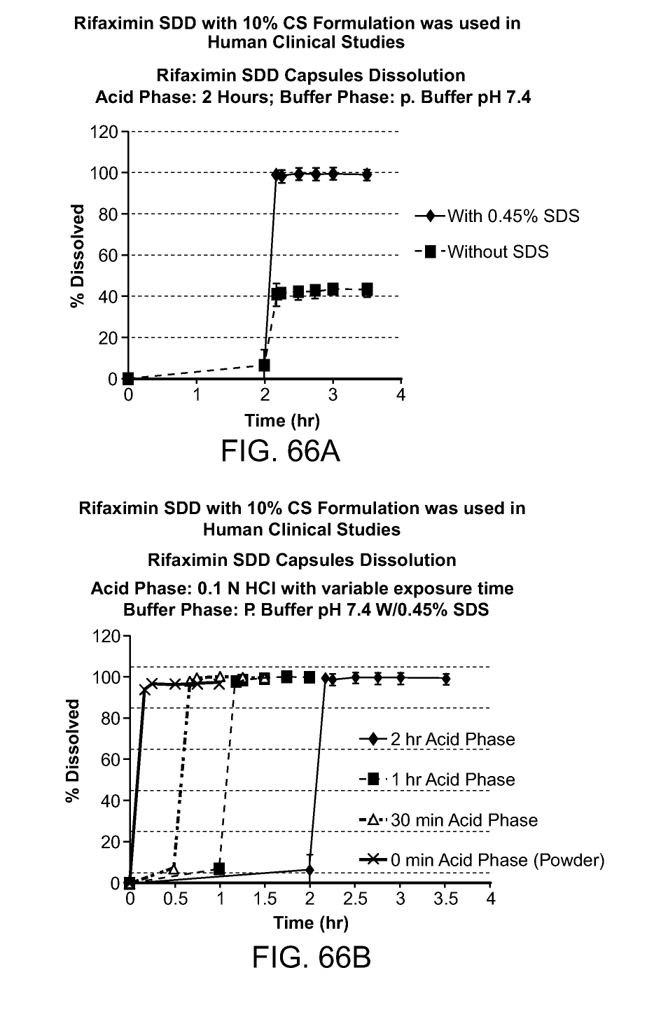

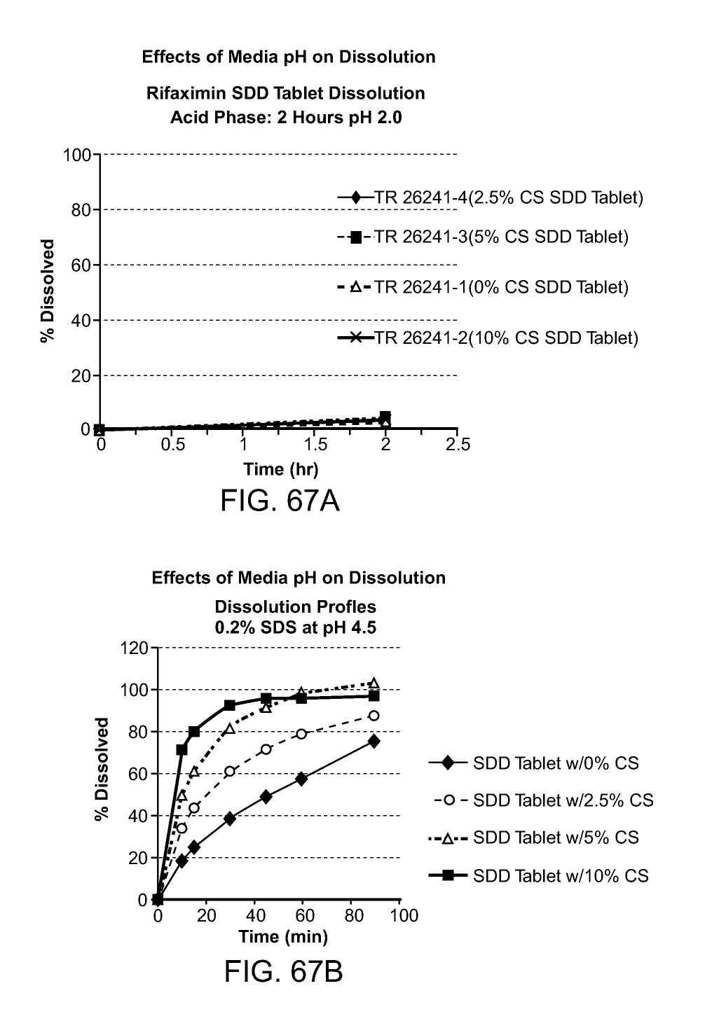

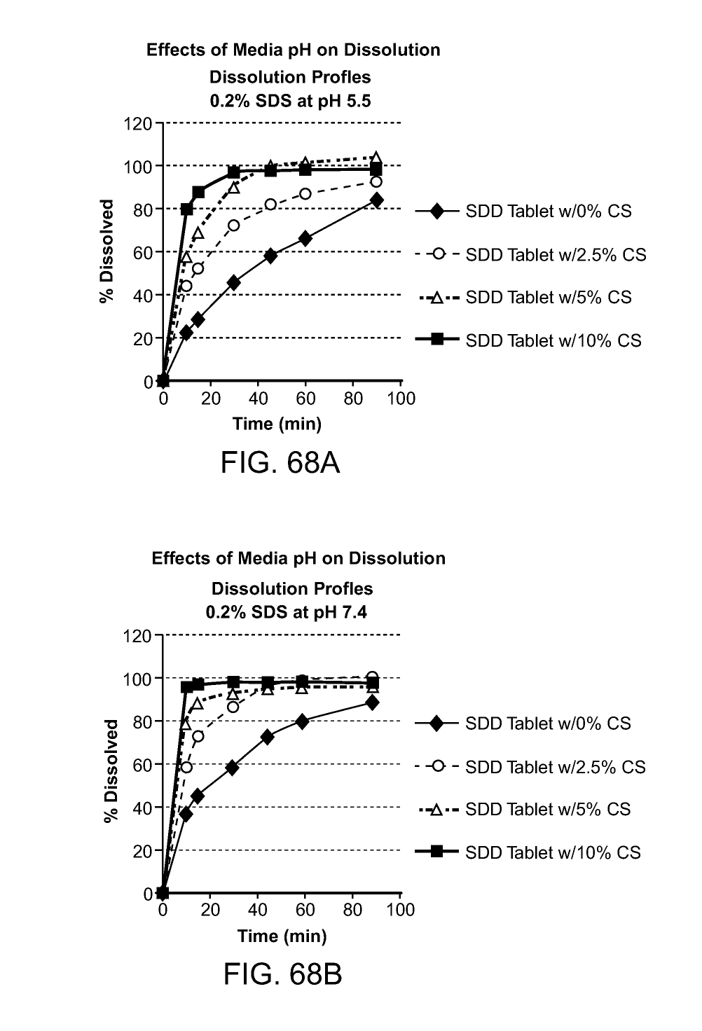

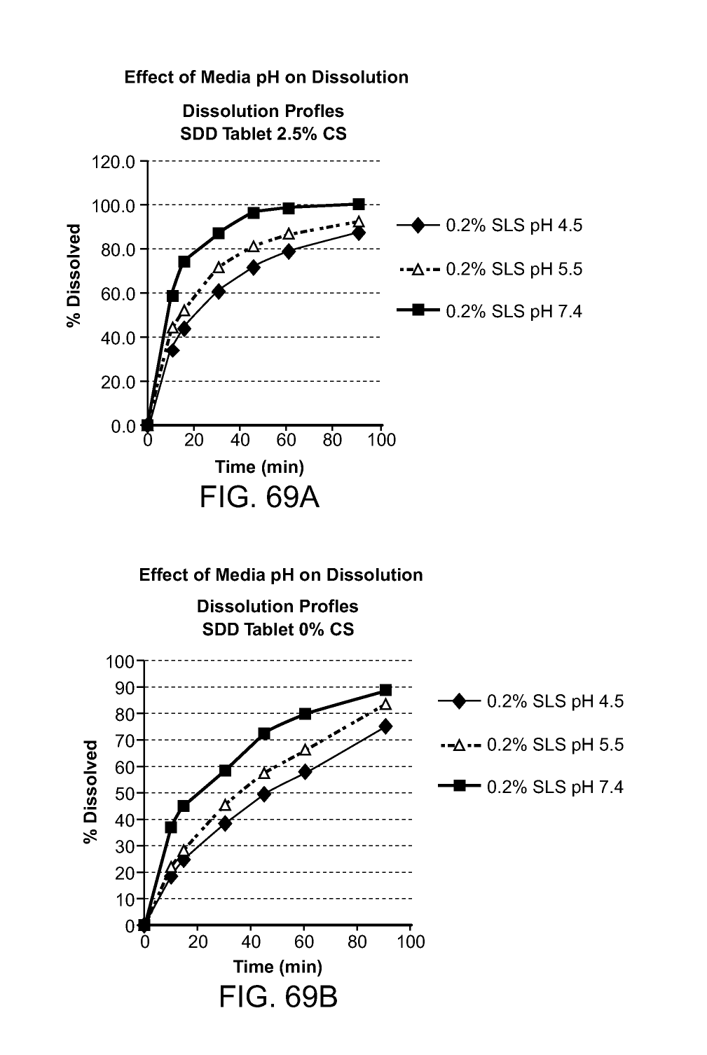

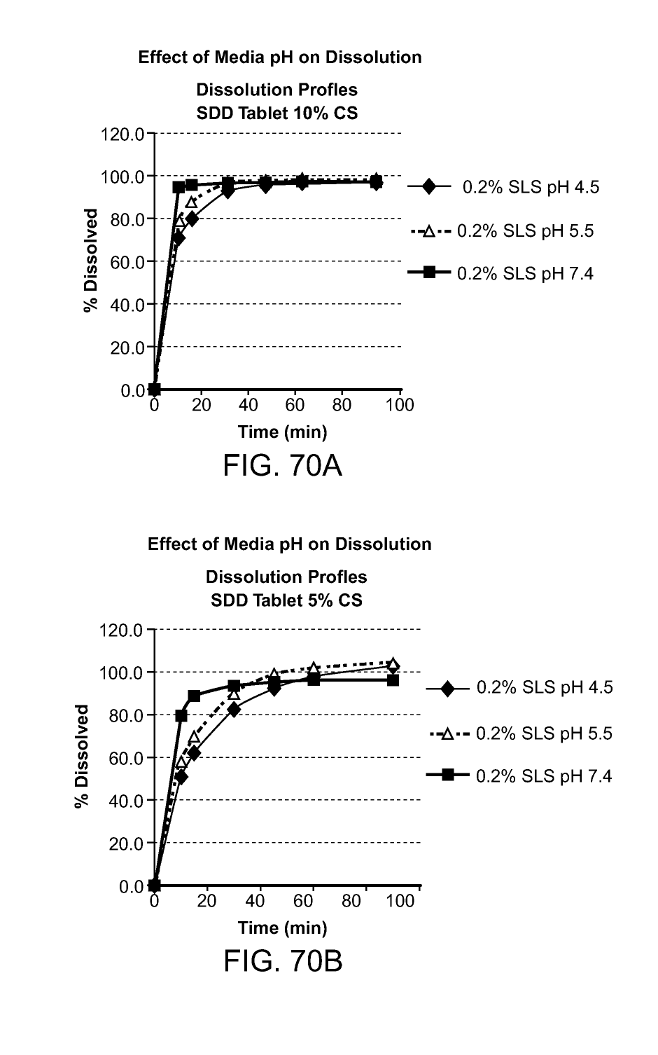

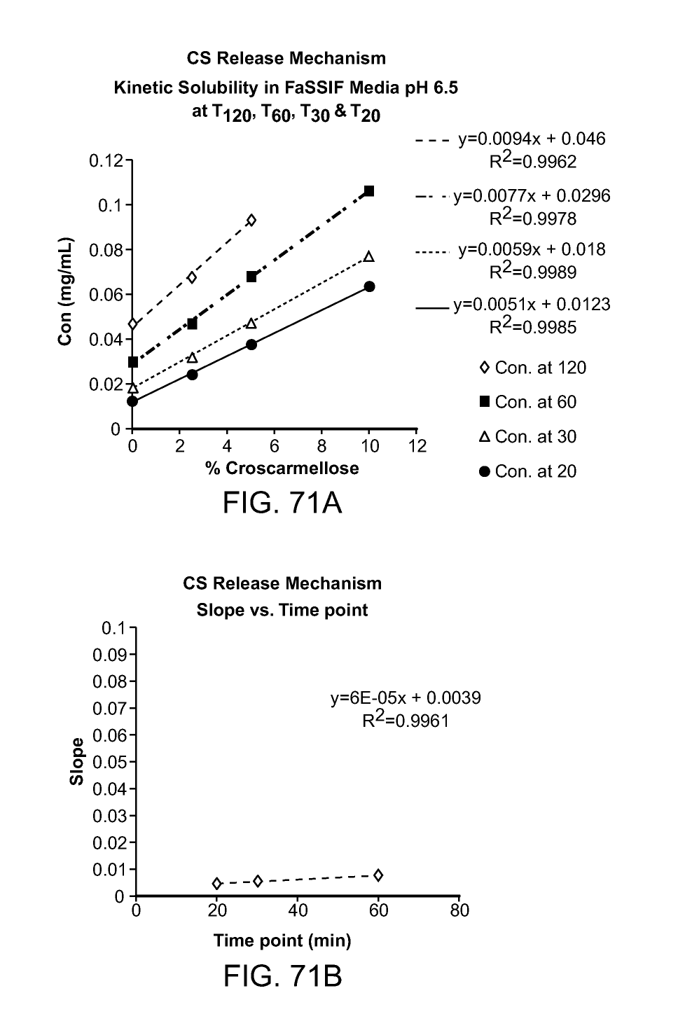

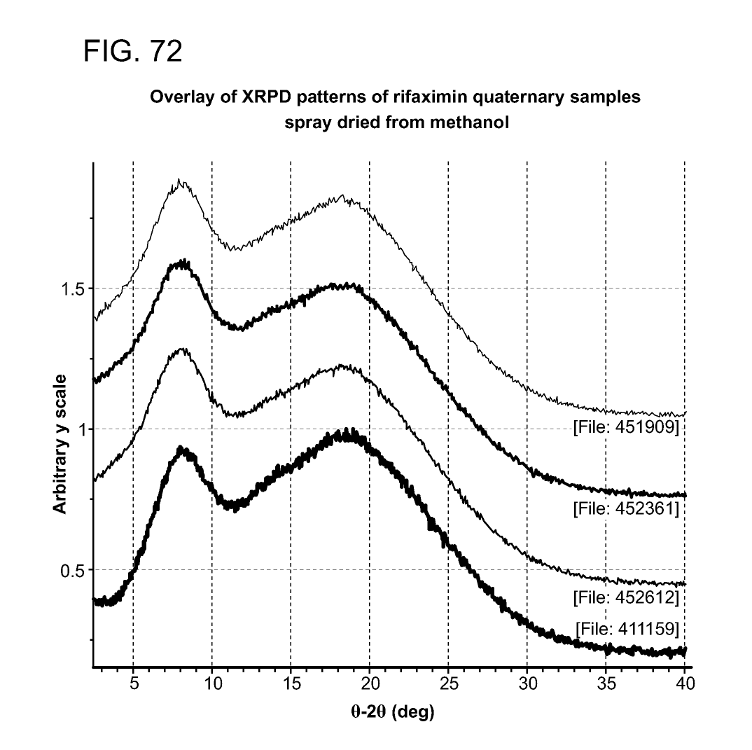

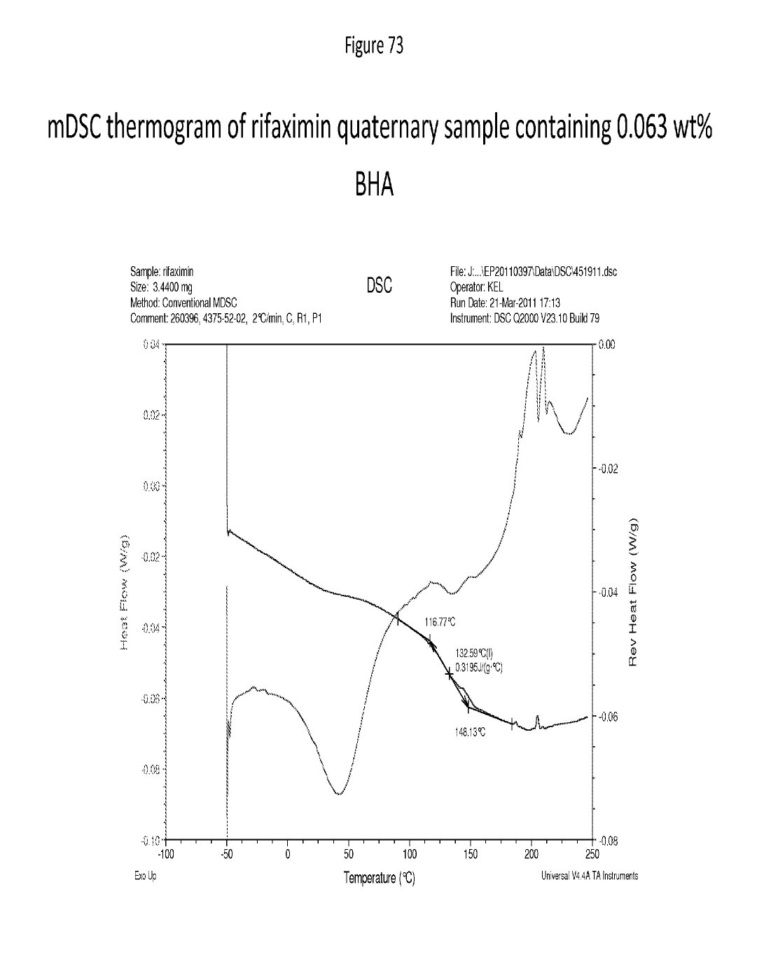

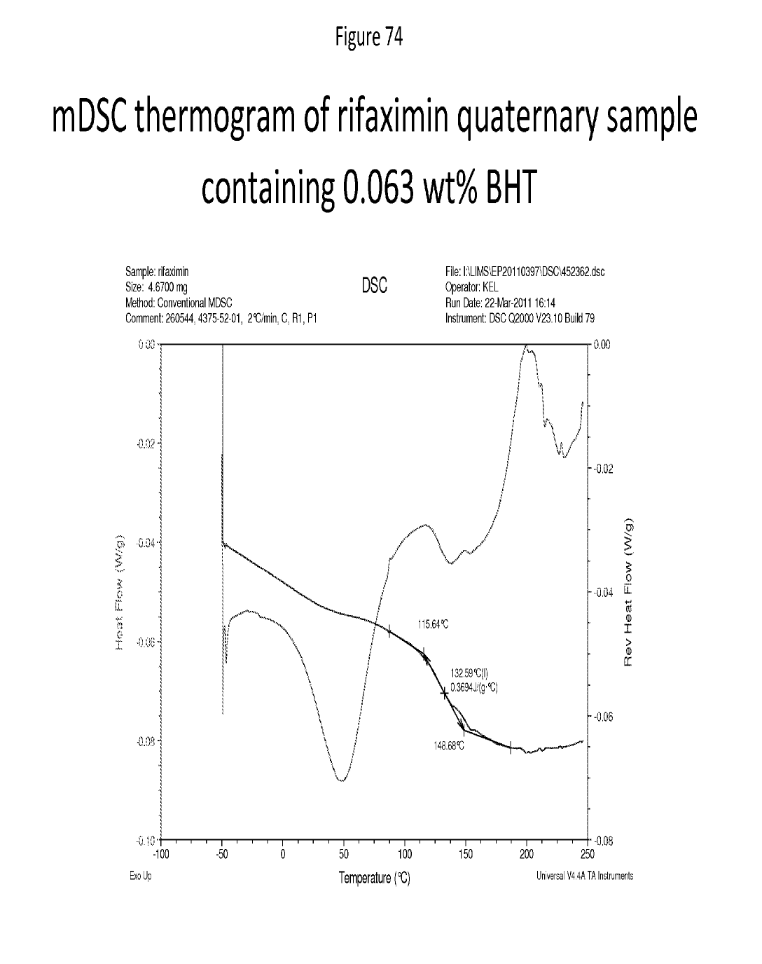

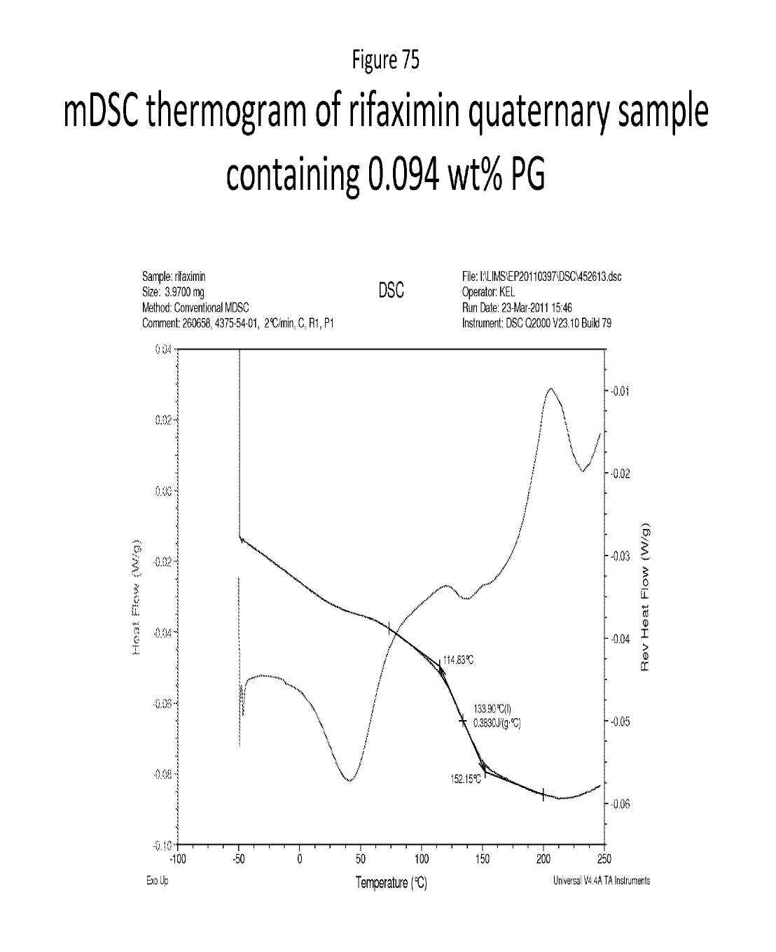

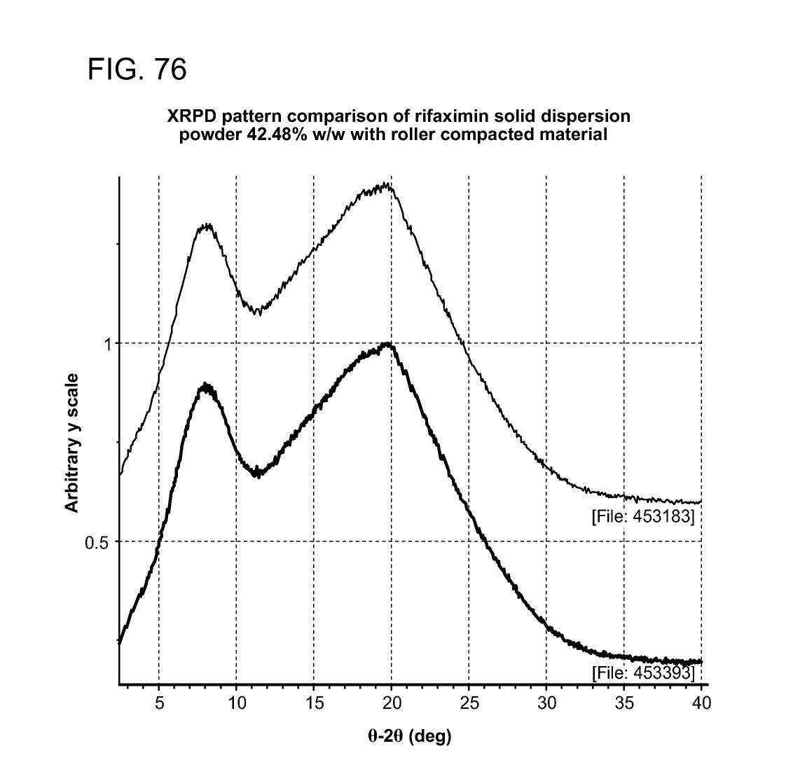

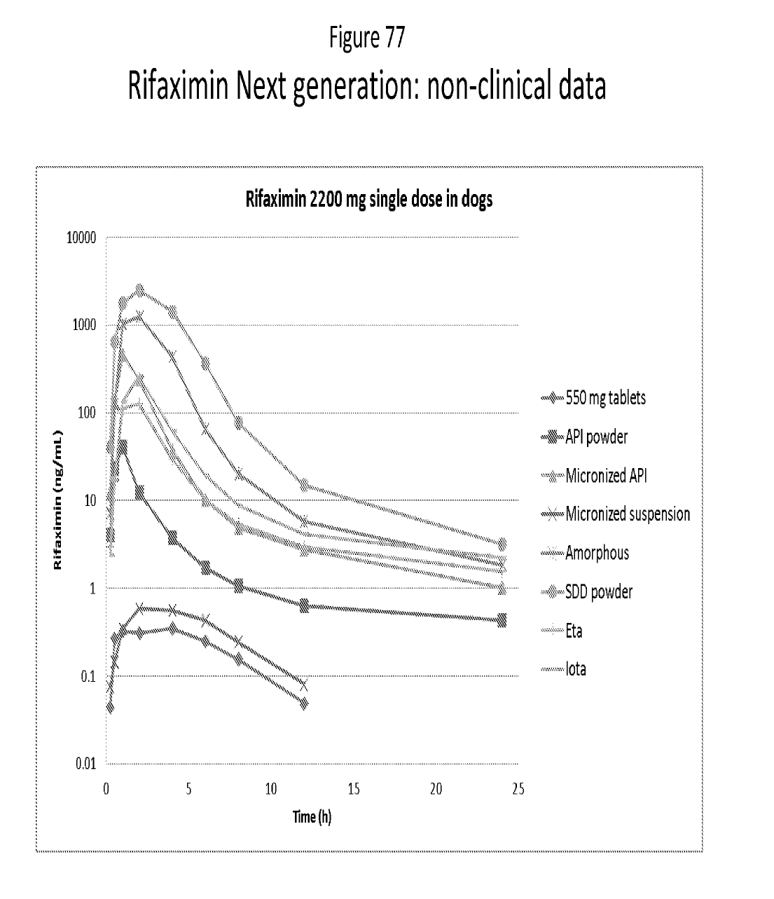

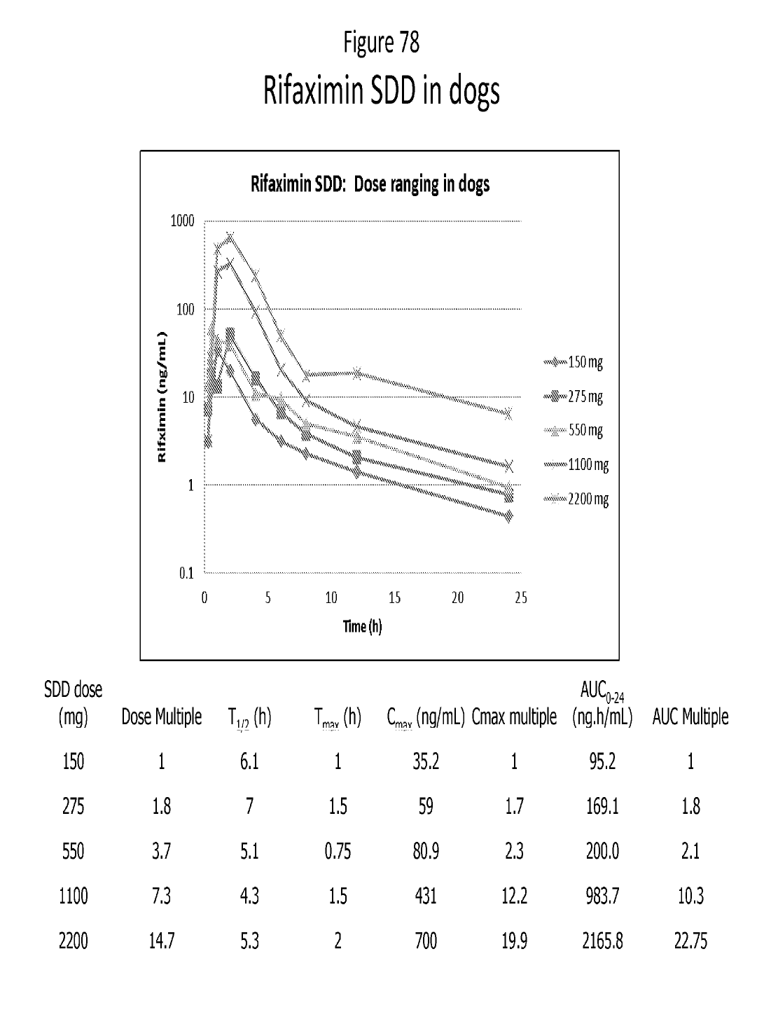

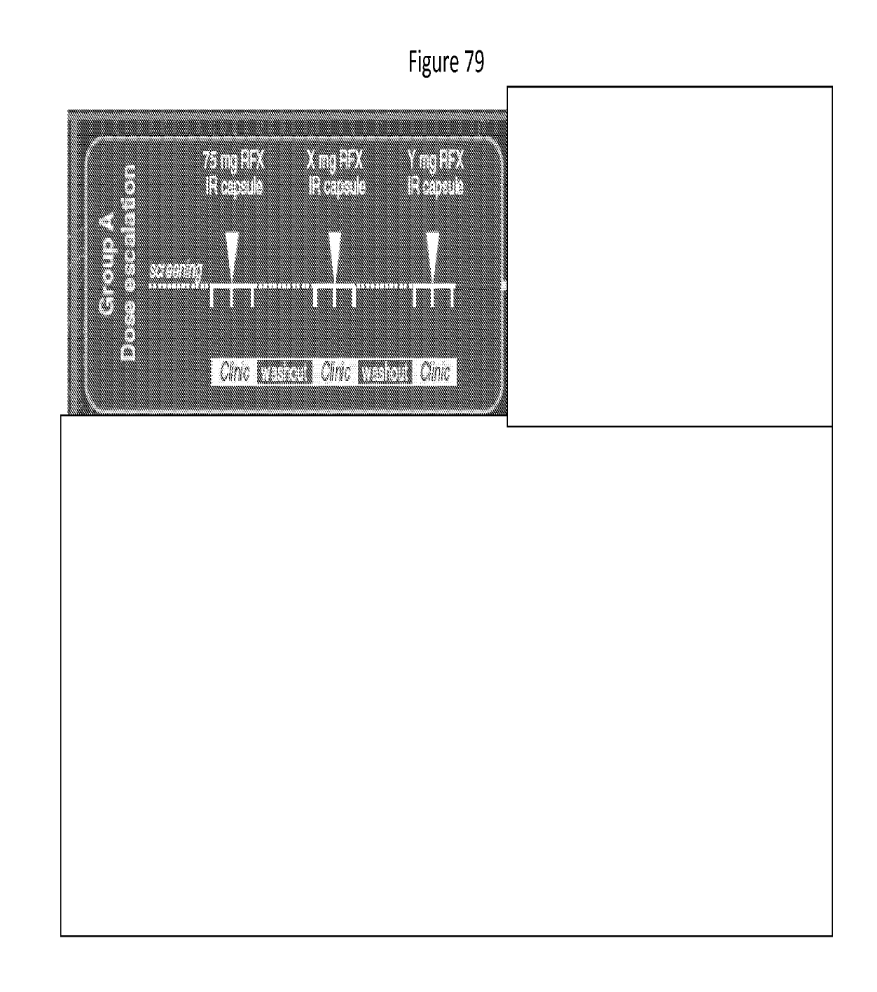

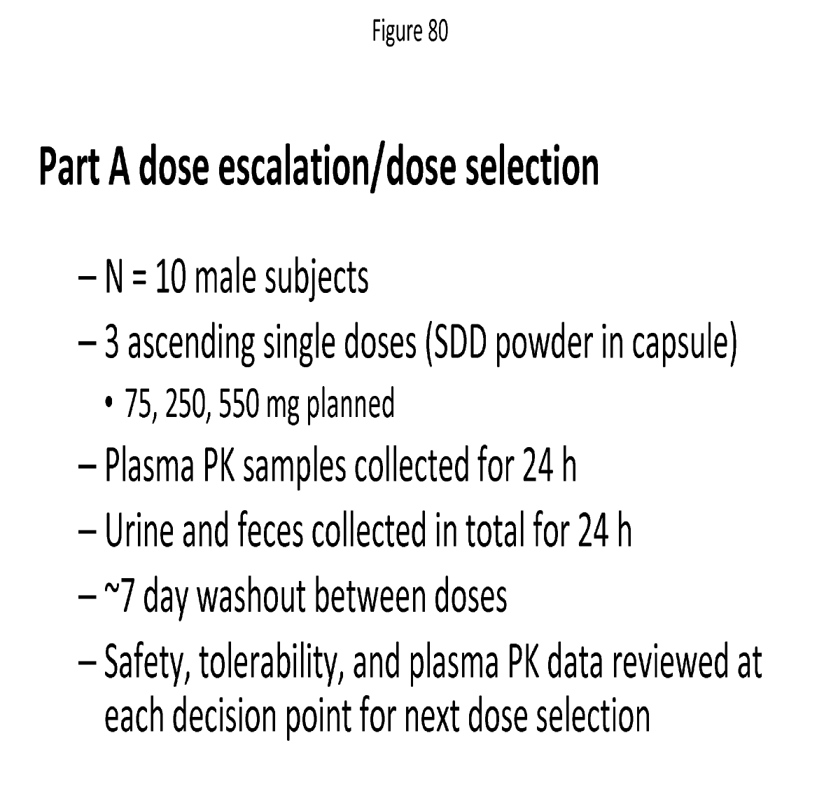

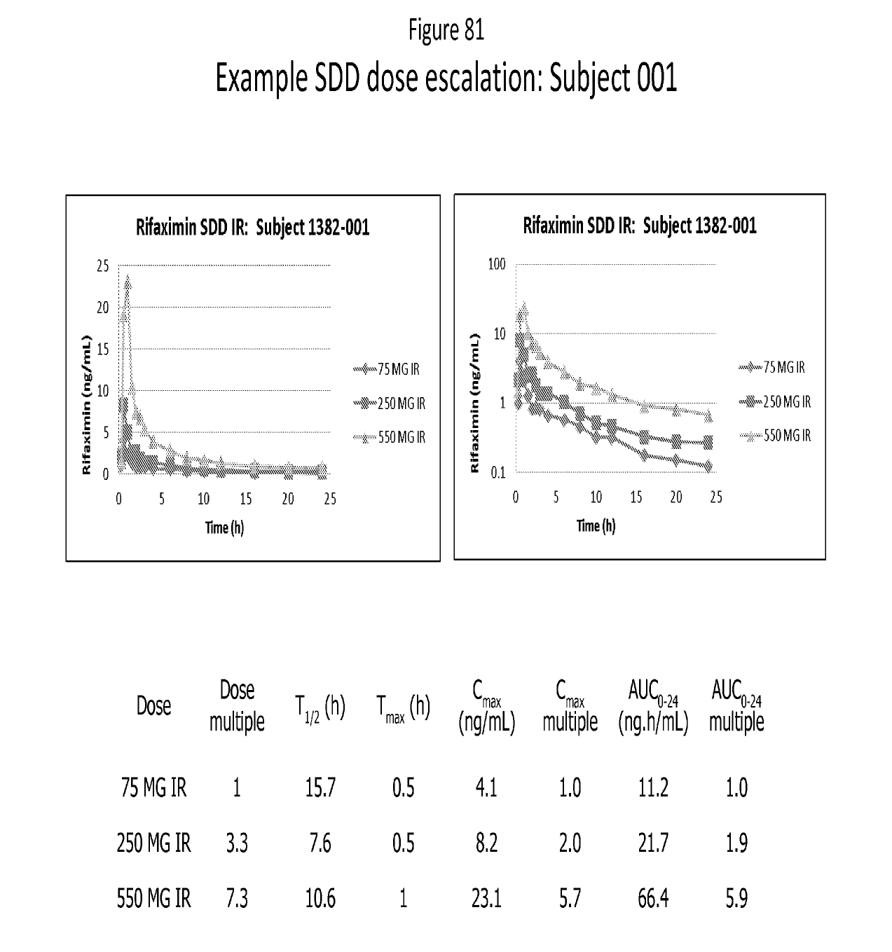

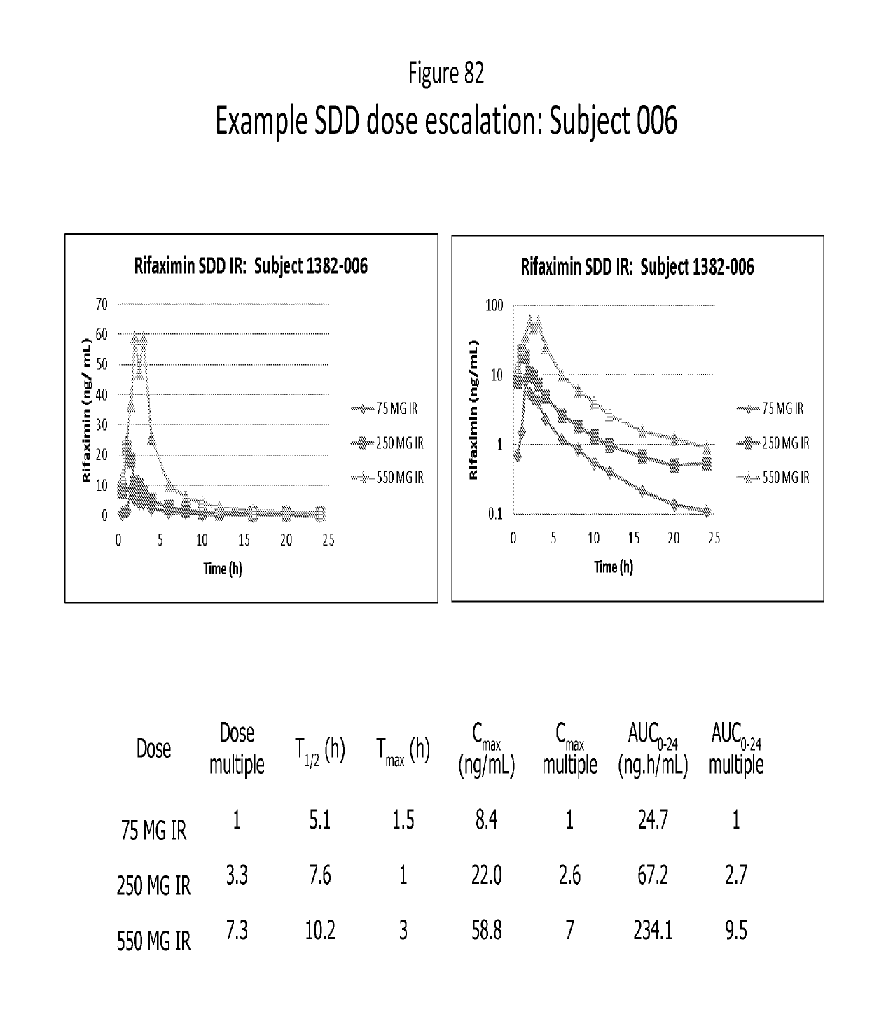

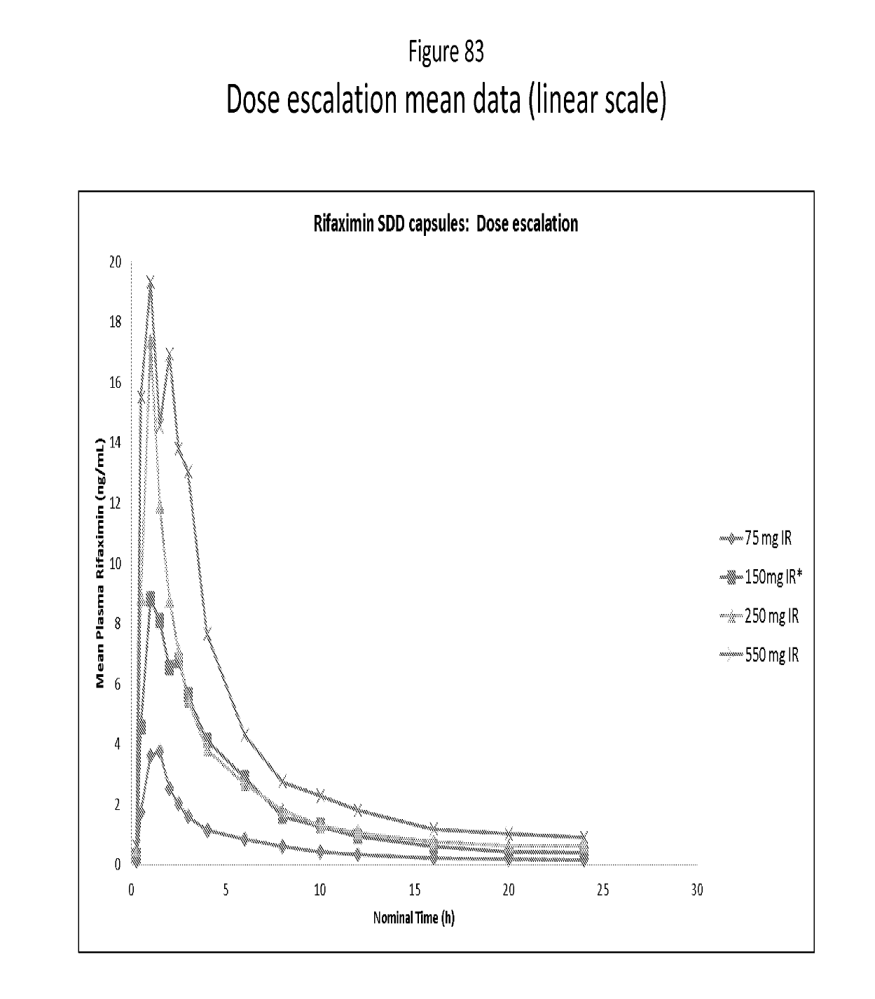

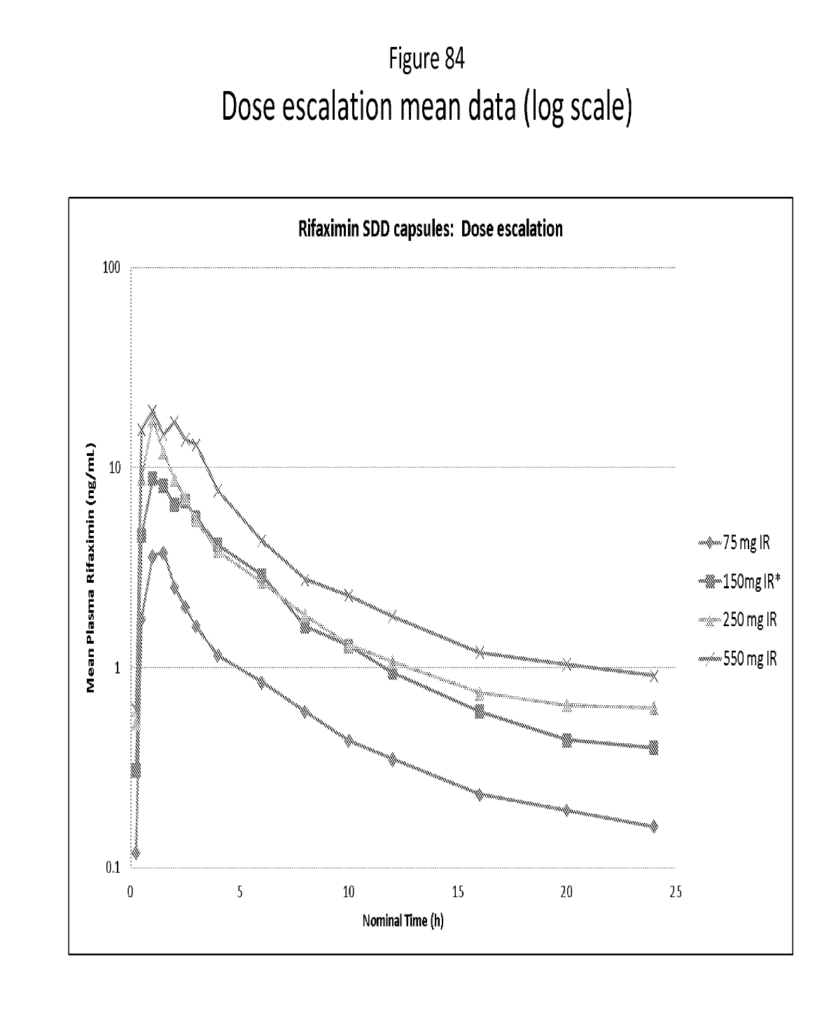

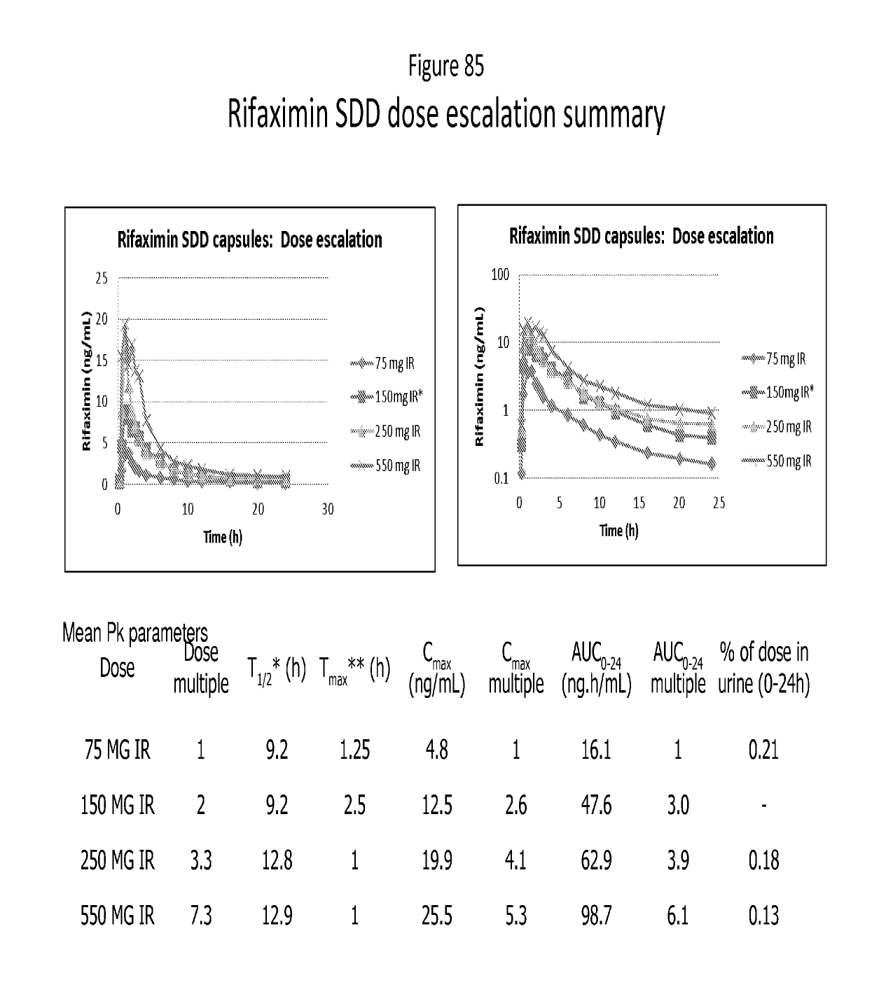

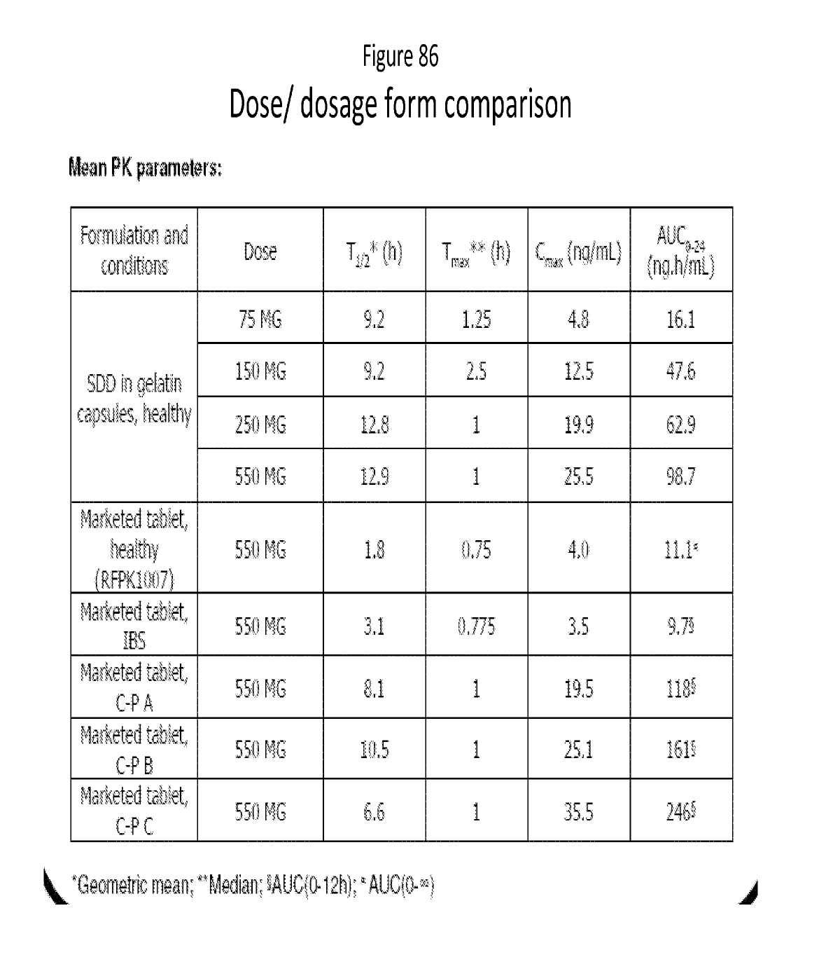

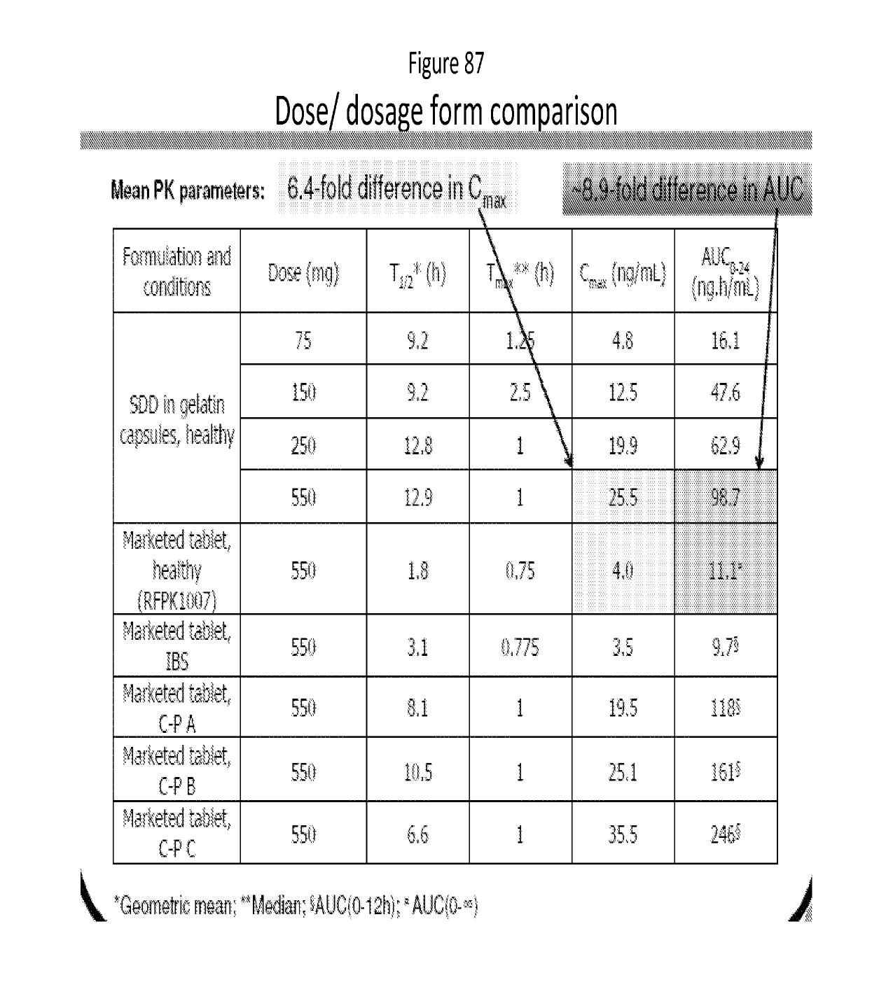

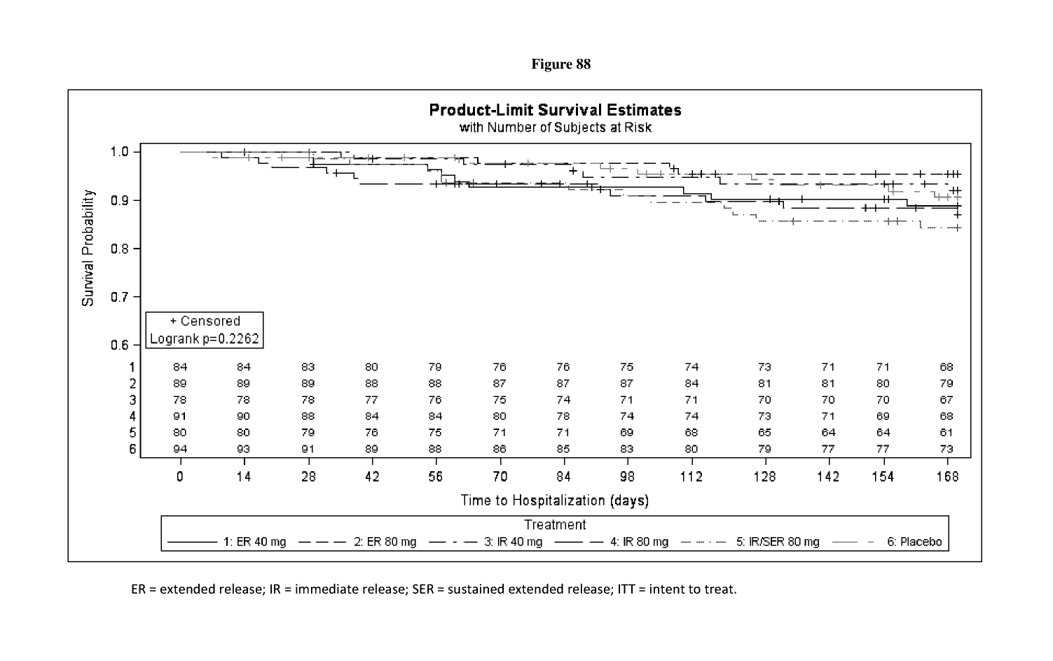

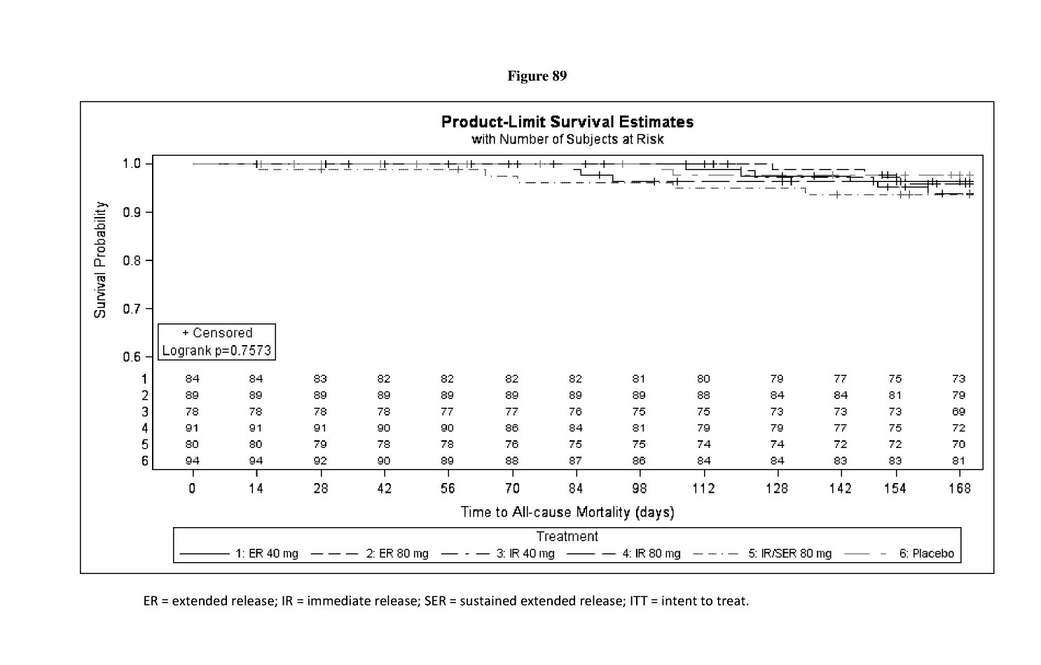

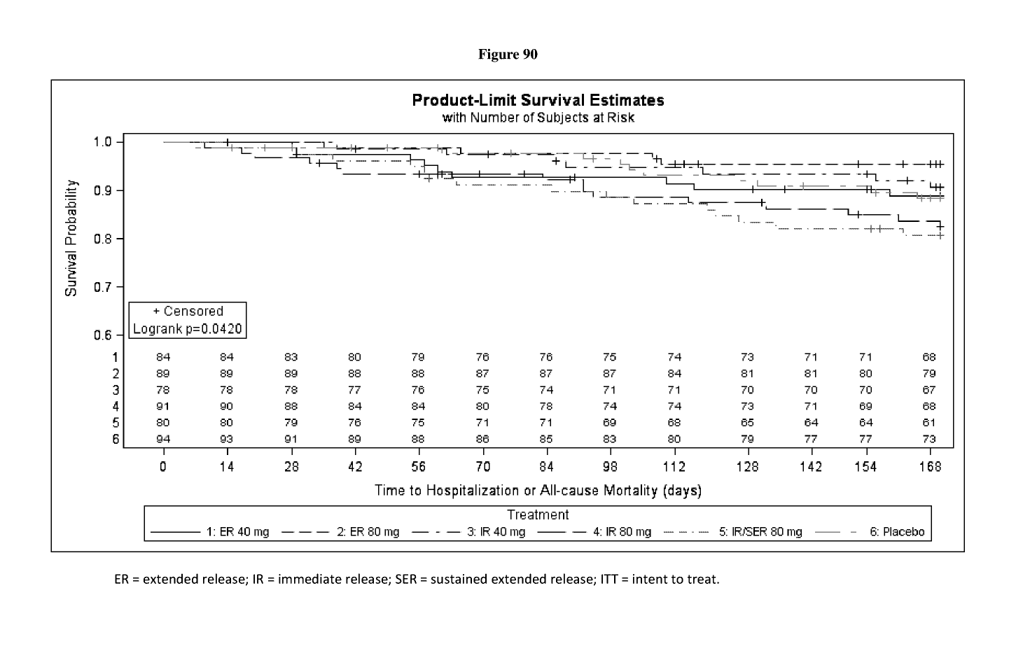

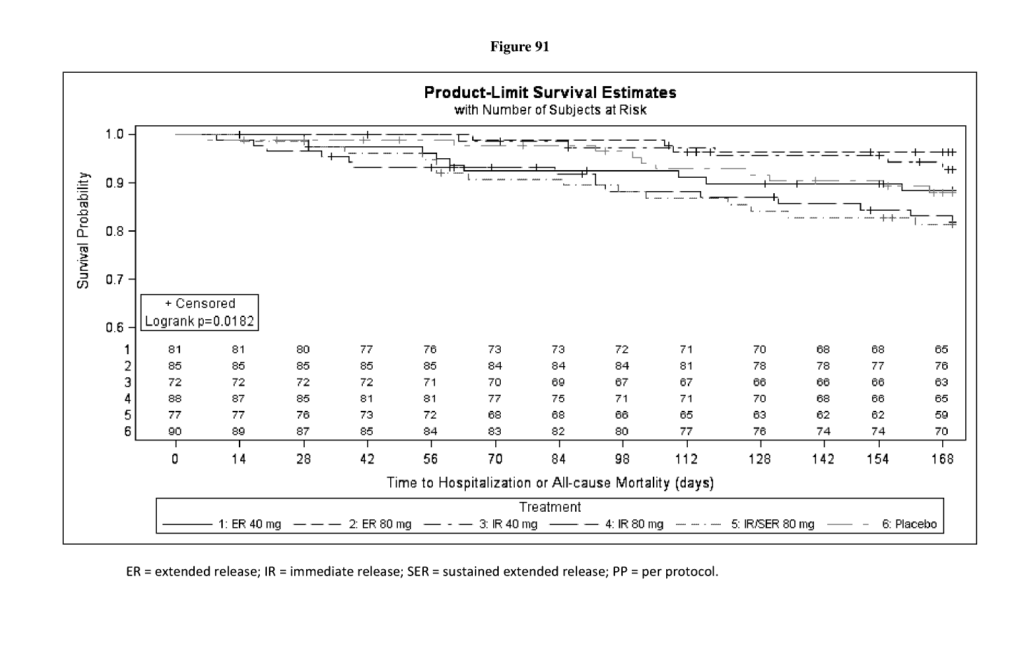

The material was then discharged into a stainless steel pan and record the height of material in the pan. Empty capsules were tared using an analytical balance, then the capsules were filled by depressing into the bed of material. The weight is adjusted within +or −5% of target fill weight of 647.5 mg (acceptable fill range 615.13-679.88 mg). FIGS. 61-63 show the rifaximin solid dispersion (SD) capsules in various buffers; with and without SDS; and compared to amorphous rifaximin. FIG. 61 shows results of dissolution studies of rifaximin SD capsules in acid phase: 0.1 N HCl with variable exposure times in a buffer containing 0.45% SDS at pH 6.8. FIG. 62 shows results of dissolution studies of rifaximin SD capsules in acid phase for 2 hours buffered at pH 6.8 with and without SDS. FIG. 63 shows results of dissolution studies of rifaximin SD capsules in acid phase in a phosphate buffer at pH 6.8 with 0.45% SDS compared to amorphous rifaximin. As shown in the FIGS. 61-63 rifaximin SD near 100% dissolution is achieved in 0.45% SDS and the SD formulation dissolves more slowly than the amorphous rifaximin.

### Example 5. Pharmacokinetic (PK) Studies of Solid Dispersion in Capsules

Presented herein are dog pharmacokinetics (PK) studies comparing various forms of rifaximin. PK following administration of rifaximin API in capsule, micronized API in capsule, nanocrystal API in capsule (containing surfactant), amorphous in capsule, and solid dispersion (SD) in capsule were tested.

In the SD dosage form, the polymer used was HPMC-AS at a drug to polymer ratio of 50:50. The formulation also comprised pluronic F127 and crosscarmellose sodium (see Example 4).

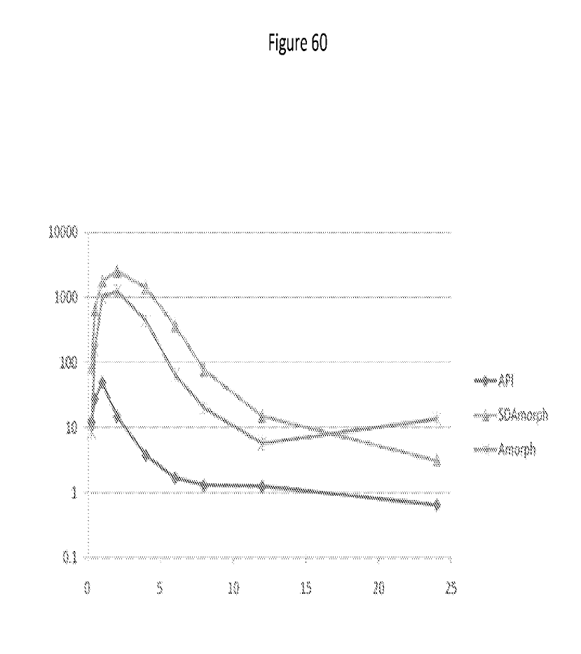

A brief study design: male beagle dogs (N=6, approximately 10 kg) received rifaximin 2200 mg in the dosage forms described above as a single dose (capsules, 275 mg, 8 capsules administered in rapid succession), in a cross-over design with one week washout between phases. Blood was collected at timed intervals for 24 h after dosage administration, and plasma was harvested for LC-MS/MS analysis. The mean concentrations are shown in FIG. 60.

Table 25 shows the PK parameters. From the table it can be seen that systemic exposure of the solid dispersion formulation is greater than that of amorphous or crystalline form (API) of rifaximin.

TABLE 25
PK Parameters of API, Amorphous and Solid Dispersion to Dogs

| | Half-life* | Tmax | Cmax | AUClast | AUCINF_obs | AUC_0-24 |
|---|---|---|---|---|---|---|
| ID | h | h | ng/mL | h * ng/mL | h * ng/mL | h * ng/mL |
| 901_API | 16.76 | 0.5 | 65.5 | 101 | 118 | 101 |
| 902_API | 9.41 | 1 | 3.83 | 25 | 29 | 25 |
| 903_API | 10.03 | 1 | 197 | 344 | 360 | 344 |
| 904_API | 3.56 | 1 | 1.21 | 5 | 6 | 6 |
| 905_API | 2.94 | 1 | 1.53 | 5 | 6 | 6 |
| 906_API | | 24 | 0.52 | 7 | | 7 |
| mean | 6.98 | 1 | 44.93 | 81 | 104 | 82 |
| SD | | [0.5-24] | 78.75 | 134 | 150 | 134 |
| 901_amorph | 5.38 | 1 | 536 | 1407 | 1421 | 1407 |
| 902_amorph | 5.93 | 2 | 4100 | 12258 | 12762 | 12258 |
| 903_amorph | 6.25 | 2 | 1050 | 3375 | 3523 | 3375 |
| 904_amorph | 4.77 | 2 | 763 | 2291 | 2306 | 2291 |
| 905_amorph | 7.72 | 1 | 1200 | 2041 | 2059 | 2041 |
| 906_amorph | 5.63 | 2 | 704 | 2076 | 2090 | 2076 |
| mean | 5.88 | 2 | 1392.17 | 3908 | 4027 | 3908 |
| SD | | [1-2] | 1348.24 | 4141 | 4334 | 4141 |
| 901_SD amorph | 6.66 | 2 | 491 | 1354 | 1394 | 1354 |
| 902_SD amorph | 2.04 | 2 | 6550 | 25140 | 25149 | 25140 |
| 903_SD amorph | 2.8 | 4 | 2410 | 10490 | 10508 | 10490 |
| 904_SD amorph | 2.24 | 1 | 1410 | 6343 | 6350 | 6343 |
| 905_SD amorph | 3.97 | 2 | 2860 | 7885 | 7895 | 7885 |
| 906_SD amorph | 4.89 | 2 | 1900 | 4532 | 4558 | 4532 |
| mean | 3.01 | 2 | 3026 | 10878 | 10892 | 10878 |
| SD | | [1-4] | 2043.58 | 8267 | 8264 | 8267 |

*geometric mean
**median and range

API exposures were low, in keeping with what has been previously observed for rifaximin. In contrast, mean exposures (AUCinf) following amorphous and SD rifaximin administration were substantially higher, with ˜40- and ˜100-fold greater exposure, respectively, as compared with API. Variability was high in all three dose groups. In general, the shapes of all three profiles were similar, suggesting effects of the dosage forms on bioavailability without effects on clearance or volume of distribution.

### Example 6. Human Clinical Studies

Rifaximin SDD with 10% CS formulation was used in human clinical studies. FIG. 65 shows the kinetic solubility of rifaximin SD granules 10% wt CS FaSSIF or 10% wt CS FeSSIF (a) and the dissolution profiles of SDD tablet 10% CS in 0.2% SLS at pH 4.5, 5.5 and 7.4. As shown in the FIG. 65, rifaximin SDD 100%, or near 100%, dissolution is achieved in 0.2% SLS, pH 4.5, 5.5 and 7.4. FIG. 66 shows that release can be delayed up to two hours and extended up to three hours.

### Example 7. Effects of Media pH on Dissolution

FIGS. 67-70 show the effects of media pH on Rifaximin SDD tablet SDD tablet dissolution at various levels of CS: 0%, 2.5%, 5%, and 10% CS. FIGS. 67 and 68 show dissolution profiles of SDD tablet with 0%, 2.5%, 5% or 10% CS in 0.2% SDS at 2 hours pH 2.0, pH 4.5, 0.2% SDS pH 5.5, or 0.2% SDS, pH 7.4. FIGS. 69 and 70 show the dissolution profiles of SDD tablet 2.5% CS, 0% CS, 10% CS and 5% CS in 0.2% SLS, pH4.5, 0.2% SLS, pH 5.5 and 0.2% SLS, pH 7.4. FIG. 71 shows CS release mechanism.

### Example 8

Described herein are the preparation and characterization of rifaximin quaternary dispersions with antioxidants. Antioxidants used were butylated hydroxyanisole (BHA), butylated hydroxytoluene (BHT) and propyl gallate (PG).

### Sample Preparation and Characterization

Three rifaximin quaternary samples were prepared by spray drying from methanol. Spray drying parameters are summarized in Table 26. Table 2 Parameters for Samples Prepared by Spray Drying

TABLE 26

| | | | | | Outlet | |
|---|---|---|---|---|---|---|
| | Inlet | | | Inlet temp. | temp. | Spray |
| Sample | temp. | | | (measured, | (measured, | rate (a) |
| ID | (set, ° C.) | Aspirator % | Pump % | ° C.) | ° C.) | mL/min |
| 0.063 wt % | 120 | 95 | 45-35 | 120-124 | 61-49 | 19 |
| of BHA in | | | | | | |
| the | | | | | | |
| dispersion | | | | | | |
| 0.063 wt % | 120 | 95 | 45-35 | 120-121 | 60-50 | 20 |
| of BHT in | | | | | | |
| the | | | | | | |
| dispersion | | | | | | |
| 0.094 wt % | 120 | 95 | 45-35 | 119-120 | 60-48 | 20 |
| of propyl | | | | | | |
| gallate in | | | | | | |
| the | | | | | | |
| dispersion | | | | | | |

(a): flow rates are estimated based on initial pump % of 45%.

TABLE 27
Characterization of Rifaximin Quaternary Samples

| Habit/Description | Analysis | Results (b) |
|---|---|---|
| orange solids, | XRPD | x-ray amorphous |
| irregular | mDSC | 133° C. (midpoint, T_{g}) |
| aggregates, no B/E | | 0.3 J/g · ° C. (ΔCp) |
| orange solids, | XRPD | x-ray amorphous |
| irregular | mDSC | 133° C. (midpoint, T_{g}) |
| aggregates, no B/E | | 0.4 J/g · ° C. (ΔCp) |
| orange solids, | XRPD | x-ray amorphous |
| irregular | mDSC | 134° C. (midpoint, T_{g}) |
| aggregates, no B/E | | 0.4 J/g · ° C. (ΔCp) |

A small sub-lot from each of spray dried materials was visually inspected by PLM and then characterized by XRPD and mDSC. Characterization results are summarized in Table 27.

The prepared materials are x-ray amorphous, as shown in FIG. 72 the overlay of XRPD patterns, which agree with their PLM observations.

In the mDSC, each of material displays a single apparent T_{g} in the reversing heat flow signal at approximately 133° C. (FIG. 73, with 0.063 wt % BHA), 133° C. (FIG. 74, with 0.063 wt % BHT), and 134° C. (FIG. 75, with 0.094 wt % PG), which is consistent with the T_{g} of the spray dried rifaximin ternary dispersion of 47.2:47.2:5.6 w/w/w/rifaximin/HPMC-AS MG/Pluronic F-127 (135 or 136° C.).

### Example 9: Rifaximin Solid Dispersions

This example sets forth exemplary microgranules of rifaximin and pharmaceutical compositions comprising the same.

Spray dry dispersion (SDD), solid dispersion, amorphous solid dispersion are used interchangeably herein to refer to the rifaximin formulations.

The complete statement of the components and quantitative composition of Rifaximin Solid Dispersion Formulation (Intermediate) is given in Table 28

TABLE 28
Composition of Rifaximin Solid Dispersion Formulation

| Component | Quantity (%) | Function |
|---|---|---|
| Rifaximin Drug | 42.48 | Active Ingredient |
| Substance | | |
| Hypromellose Acetate | 42.48 | Solubility Enhancer |
| Succinate (HPMC-AS) | | |
| Poloxamer 407 | 5.04 | Surfactant |
| Croscarmellose | 10.00 | Dissolution Enhancer |
| Sodium | | |

### Composition of Rifaximin Solid Dispersion IR Capsule

TABLE 29
Composition of Rifaximin solid dispersion IR capsule

| Component | Quantity | Function |
|---|---|---|
| Rifaximin solid dispersion | 75 mg-275 mg* | Active ingredient |
| (amorphous) | | |
| Hard Gelatin capsules | 1 unit | Capsule |
| Coni-Snap, Size 000, | | |
| Transparent | | |

*Rifaximin dose equivalent

Description of Manufacturing Process and Process Controls

### Manufacturing Process for Rifaximin Solid Dispersion Formulation

Table 30 sets forth the manufacture of Rifaximin solid dispersion microgranules

TABLE 30

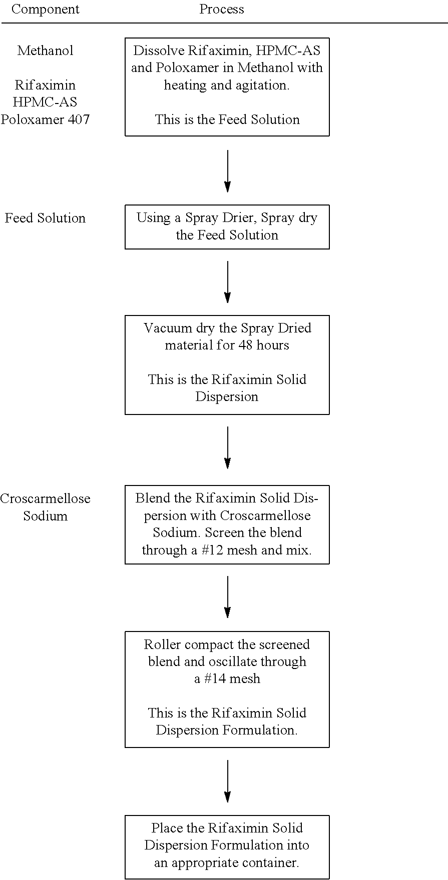

### Manufacturing Process for Rifaximin Solid Dispersion IR Capsules

The manufacturing process the Rifaximin solid dispersion IR capsules is given in Table 31.

TABLE 31

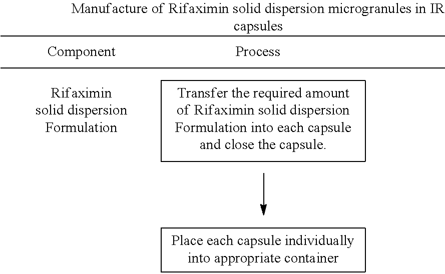

Exemplary spray drying processes are set forth in Table 32.

TABLE 32
Spray Drying Process:
Spray Dryer - PSD 1
Two Fluid Niro Nozzle
Nozzle orifice - 1 mm
Inlet gas temperature - 125 ± 3 deg C.
Process gas flow (mmH2O) - 44
Atomizing gas pressure - 1 bar
Feed rate - 4.7 kg/Hr
Outlet temperature - 55 ± 3 deg C.
Solution temperature - 36 deg C.
Post spray drying vacuum dry at 40 deg C. for 48 hrs

| | Micronized | | API | | Amorphous | | Amorphous | | Micronized | |
|---|---|---|---|---|---|---|---|---|---|---|
| | Caps | | Caps | | Caps | | SD caps | | Tab | |
| Ingredients | % | g/dose | % | g/dose | % | g/dose | % | g/dose | % | g/dose |
| Rifaximin | 95.5 | 2.2 | 47.2 | 2.2 | 51.7 | 2.2 | 42.47 | 2.2 | 50 | 2.2 |
| Ac-di-sol | 4.5 | 0.1 | 5 | 0.23 | 5 | 0.21 | 10.02 | 0.52 | 7.5 | 0.33 |
| Mannitol | | | 47.8 | 2.23 | 43.3 | 1.84 | | | | |
| 160C | | | | | | | | | | |
| Pluronic | | | | | | | 5.04 | 0.26 | | |
| 188 | | | | | | | | | | |
| HPMC AS | | | | | | | 42.47 | 2.2 | | |
| Avicel 113 | | | | | | | | | 26 | 1.14 |
| Avicel 112 | | | | | | | | | 15 | 0.66 |
| Magnesium | | | | | | | | | 1 | 0.04 |
| Stearate | | | | | | | | | | |
| Cab-o-sil | | | | | | | | | 0.5 | 0.02 |
| Avicel CL- | | | | | | | | | | |
| 611 | | | | | | | | | | |
| Mannitol | | | | | | | | | | |
| 160C | | | | | | | | | | |
| Total | 100 | 2.3 | 100 | 4.66 | 100 | 4.26 | 100 | 5.18 | 100 | 4.4 |

### Example 10: Characterization of Drug Product Samples Containing Rifaximin Solid Dispersion

Disclosed herein is dissolution data for roller compacted materials of Solid Dispersion Rifaximin with varying levels (0, 2.5%, 5%, and 10%) of croscarmellose sodium.

Three roller compacted material of Amorphous Solid Dispersion Rifaximin with varying levels (0, 2.5%, 5%) of croscarmellose sodium were dissolution tested. Results are compared to dissolution of the rifaximin granules with 10% croscarmellose sodium.

### Dissolution Studies with USP Paddle Method

Dissolution tests were performed on as received roller compacted materials of Solid Dispersion Rifaximin with 0, 2.5 wt %, and 5 wt % croscarmellose sodium. Powders of solids were directly added into pH 6.5 FaSSIF buffer with gentle agitation of the media (50 rpm paddle stirrer) at 37° C. for 24 hrs.

At designated time points of 5, 10, 20, 30, 60, 90, 120, 240 and 1440 minutes, aliquots were removed from each of the samples. Analysis of the date indicates that an increase in rifaximin concentration is apparent with the rising croscarmellose sodium level in materials, particularly in the early stage of the dissolution. After 24 hrs, the rifaximin concentration from granules containing 5 wt % croscarmellose sodium is similar to granules with 10 wt % croscarmellose sodium.

### Example 11: Characterization of Rifaximin Solid Dispersion Powder 42.48% w/w

Described herein is the characterization of Rifaximin Solid Dispersion Powder 42.48% w/w. Dissolution testing was also performed on the material at pH 6.5 in FaSSIF at 37° C.

A sample of rifaximin ternary dispersion was characterized by XRPD, mDSC, TG-IR, SEM and KF.

X-ray powder diffraction (XRPD) analysis using a method for Rifaximin Solid Dispersion Powder 42.48% w/w was conducted. The XRPD pattern by visual inspection is x-ray amorphous with no sharp peaks (FIG. 76). By mDSC a single apparent T_{g} is observed from the step change in the reversing heat flow signal at approximately 134° C. with a heat capacity change at T_{g} of approximately 0.36 J/g·° C.

Thermogravimetric analysis coupled with infra-red spectroscopy (TG-IR) was performed to analyze volatiles generated upon heating. The total weight loss of sample was approximately 0.4 wt % to 100° C., and a dramatic change in the slope occurs at approximately 190° C. which is likely due to decomposition. The Gram-Schmidt plot corresponds to the overall IR intensity associated with volatiles released by a sample upon heating at 20° C./min Gram-Schmidt indicates that volatiles are released upon heating after ˜8 min, and volatiles were identified as residual methanol from the processing solvent in spray drying and possible acetic acid from HPMC-AS MG.

KF analysis indicates that the material contains 1.07 wt % water [(1.00+1.13)/2=1.07%].

### Example 12: Methods for Spray Drying Rifaximin Ternary Dispersion (50:50 w/w Rifaximin:HPMC-AS MG with 5.9 wt % Pluronic F-127)

Provided herein are procedures to spray dry Rifaximin ternary dispersion (50:50 w/w Rifaximin. HPMC-AS MG with 5.9 wt % Pluronic F-127).

Rifaximin ternary dispersions (50:50 w/w Rifaximin. HPMC-AS MG with 5.9 wt % Pluronic F-127) were prepared from methanol using Büchi B-290 Mini Spray Dryer in closed mode suitable for processing organic solvents. Ingredients are listed in Table 33 below:

TABLE 33

| No. | Component | mg/g | Purpose |
|---|---|---|---|
| 1 | Rifaximin | 472 | active pharmaceutical ingredient |
| 2 | Hydroxypropylmethyl | 472 | stabilizing agent |
| | cellulose acetate | | |
| | succinate (HPMC-AS), | | |
| | Type MG | | |
| 3 | Pluronic F-127 | 56 | wetting agent |
| 4 | Methanol | — | volatile; removed during process |

Rifaximin ternary dispersions were prepared by spray drying in both small scale (˜1 g API) and large scale (≧34 g API in a single batch).

For a small-scale sample, rifaximin and then the methanol were added into a clean flask. The mixture was stirred at ambient for ˜5 min to give a clear solution. HPMC-AS MG and Pluronic F-127 were added in succession and the sample was stirred for ˜1 hr. An orange solution was obtained.

For a large-scale sample, a solution was prepared at ˜40° C. Rifaximin and then methanol were added to a clean flask and the mixture was stirred at ˜40° C. for ˜5 min until clear. HPMC-AS MG, and then Pluronic F-127 were added into the rifaximin solution under stirring at ˜40° C. The sample continued to stir for ˜1.5 hr to 2 hr at this temperature. A dark red solution was obtained. The sample was removed from the hot plate and left at ambient to cool.

Experimental conditions to prepare Rifaximin ternary solutions are summarized in Table 34 below:

TABLE 34

| | weight | | |
|---|---|---|---|
| | (API/HPMC AS MG/ | Temper- | Concentration |
| Solvent | Pluronic F127, g) | ature | (g/L) |
| methanol, 100 mL | 1.0535/1.0529/0.1249 | ambient | 22.3 |
| methanol, 1000 mL | 34.07/34.07/4.02 | ~40° C. | 72.2 |
| methanol, 1250 mL | 50.34/50.32/5.94 | ~40° C. | 85.3 |
| methanol, 1250 mL | 50.16/50.14/5.92 | ~40° C. | 85.0 |
| methanol, 1250 mL | 50.05/50.06/5.91 | ~40° C. | 85.0 |

During spray drying process, both the small and large scale rifaximin ternary solutions were kept at ambient temperature. The Pump % was decreased during process in attempt to control outlet temperature above 40° C. The operating parameters used for processing are presented in Table 35 below.

TABLE 35

| | Inlet | | | Inlet temp. | Outlet temp. | Spray rate |
|---|---|---|---|---|---|---|
| | temp. | | | (measured, | (measured, | (b) |
| Description (a) | (set, ° C.) | Aspirator % | Pump % | ° C.) | ° C.) | mL/min |
| 50:50 | 120 | 95 | 35 | 120 | 60-55 | 10.4 |
| Rifaximin:HPMC-AS | 120 | 95 | 65-30 | 120-119 | 61-42 | 23 |
| MG | 120 | 95 | 50-30 | 120-119 | 67-43 | 16 |
| 5.9 wt % Pluronic F- | 120 | 95 | 50-30 | 120-119 | 65-43 | 16 |
| 127 | 120 | 95 | 50-30 | 120-119 | 67-43 | 16 |

(a): 50:50 is approximate ratio of Rifaximin to polymer, by weight; 5.9 wt % Pluronic is weight fraction to 50:50 Rifaximin:HPMC-AS MG dispersion.
(b): flow rates are estimated. Flow rate for 4103-41-01 was measured at pump 35%; for 4103-56-01 was measured at pump 65%, while for others were measured at pump 50%.

Solids recovered after spray drying were dried at 40° C. under vacuum for 24 hours and then stored at sub-ambient (freezer) over desiccant.

### Example 13. Non-Clinical Data-Form/Formulation Comparison and Dose Ranging in Dogs

Described herein is non-clinical data, form/formulation comparison in dogs and SDD dose ranging in dogs. FIG. 77 indicates the results of two studies conducted to characterize the pharmacokinetics of rifaximin following administration of varying forms and formulations following a single oral dose. Blood samples were collected at timed intervals over the 24 h after single dose administration (2200 mg total dose in each case) and processed to plasma for analysis of rifaximin concentrations. PK parameters were estimated by noncompartmental methods. The results are shown in FIG. 77. Of the forms/formulations shown, the spray-dried dispersion showed that the highest exposure, and therefore the highest bioavailability, resulted from administration of the SDD formulation (dosed as SDD powder in gelatin capsules). In order of decreasing exposure among forms dosed in gelatin capsule formulation, SDD>amorphous>iota>micronzed>eta>current crystalline API. Lower in systemic exposure than all of those are the micronized suspension formulation (reconstituted powder for oral suspension) and the current 550 mg Xifaxan tablet. Table 36, below, shows Pk parameters for dog forms.

TABLE 36

| | Tmax | Cmax | AUCall | AUCINF_obs | HL_Lambda_z |
|---|---|---|---|---|---|
| | h | h | ng/mL | h*ng/mL | h*ng/mL |
| Eta | 9.70 | 1.5 | 162.28 | 434.14 | 608.14 |
| Iota | 6.56 | 2 | 276.50 | 718.23 | 739.94 |
| Amorphous | 5.82 | 2 | 1392.17 | 3907.84 | 4026.86 |
| API capsules | 5.64 | 1 | 44.93 | 81.20 | 103.83 |
| SDD | 3.16 | 2 | 2603.50 | 9290.71 | 9308.83 |
| Micronized | 8.10 | 1 | 473.43 | 894.65 | 905.97 |
| capsules | | | | | |
| Micronized | 5.22 | 3 | 0.68 | 5.11 | 8.41 |
| suspension | | | | | |
| Micronized | 4.77 | 5 | 0.83 | 6.81 | 10.20 |
| tablets | | | | | |
| Nanocrystal | 5.01 | 5 | 0.99 | 9.05 | 8.70 |
| capsules | | | | | |

FIG. 78 shows the results of the dog dose escalation, in which dogs received single doses of the SDD formulation in capsules, at doses from 150 mg to 2200 mg. The results indicate an essentially linear dose escalation (increases in exposure that are approximately proportional to increase in dose) up to 550 mg, followed by a greater-than-proportional increase at 1100 mg and 2200 mg. This is quite unusual in the linear range in that the current crystalline form of rifaximin does not dose escalate, generally, exposure does not increase substantially on increasing dose. The greater than dose proportional increase on increasing dose is also remarkable and suggests that, at the higher doses, rifaximin is saturating intestinal P-glycoprotein transport that would otherwise limit systemic absorption, thereby allowing increased absorption.

### Example 14. Human Studies

Described herein are clinical studies carried in ten male human subjects. FIG. 79 sets out the quotient study design for rifaximin SDD dose escalation. FIG. 80 outlines the dose escalation/regional absorption study, dose escalation/dose selection. FIGS. 81 and 82 show representative subject data from an exemplary dose escalation study. Mean data (linear scale and log scale) is shown in FIGS. 83 and 84, respectively. Mean profiles, log scale. Terminal phases are parallel, in clearance mechanisms. A summary of rifaximin SDD dose escalation is shown indicating that it is likely that there is not saturation of any metabolic or other systemic FIG. 85. To summarize, there are roughly dose proportional increases in exposure (Cₘₐₓ and AUC) with increases in dose, as shown by Cₘₐₓ multiple and AUC multiple columns Tₘₐₓ is not delayed by dose increases, further indicating an early absorption window (corroborated by regional absorption data). The percent of dose in urine is remarkable in that it stays low, approximately 0.2% or less of the dose excreted over 24 h. This result is surprising in that this is quite low in spite of the significant increases in systemic exposure as compared with the crystalline formulation. Taken together, the results indicate a considerably increased solubility that presumably leads to increased local/lumenal soluble rifaximin, with accompanying increases in systemic exposure, but without significant increases in urinary excretion that are reflective of percent of rifaximin dose absorbed.

Dose/dosage form comparisons are shown in FIGS. 86 and 87. The tables compare SDD at increasing doses to the current crystalline formulation in terms of systemic PK. As noted in FIG. 87, as compared to the PK of rifaximin from the current formulation, the SDD formulation at the same dose shows an approximate 6.4-fold increase in Cₘₐₓ and an approximate 8.9-fold increase in AUC. Nonetheless, these exposures are less than those observed in any hepatic impaired subject with the current tablet formulation.

### Example 15. Exemplary Tablet Formulations

According to certain exemplary embodiments, microgranules, blends and tablets are formulated as set forth in Table 37, below

TABLE 37
Rifaximin SDD Granules

| | | % w/w | % w/w | % w/w | % w/w |
|---|---|---|---|---|---|
| | | (0% | (2.5% | (5% | (10% |
| Component | Function | CS) | CS) | CS) | CS) |
| Rifaximin | Drug | 47.2 | 46.02 | 44.84 | 42.48 |
| HPMC-AS | Polymer | 47.2 | 46.02 | 44.84 | 42.48 |
| Pluronic | Wetting | 5.6 | 5.46 | 5.32 | 5.04 |
| F-127 | agent | | | | |
| Croscarmellose | Rate | 0 | 2.5 | 5 | 10 |
| Na (CS) | controlling | | | | |
| | Total | 100 | 100 | 100 | 100 |

Granule Blend

| | | mg/Tab | mg/Tab | mg/Tab | mg/Tab |
|---|---|---|---|---|---|
| Roller Compacted | Granules | 635.59 | 652.34 | 669.05 | 706.21 |
| Granules | | | | | |
| Avicel PH102 | Filler | 166 | 149.18 | 132.52 | 95.38 |
| Croscarmellose | Disintegrant | 42.5 | 42.5 | 42.5 | 42.5 |
| Na [Extra- | | | | | |
| granular] | | | | | |
| Cab-O-Sil | Glidant | 1.7 | 1.7 | 1.7 | 1.7 |
| Magnesium | Lubricant | 4.25 | 4.25 | 4.25 | 4.25 |
| Stearate | | | | | |
| | Total | 850.04 | 849.97 | 850.02 | 850.04 |

Overall Rifaximin Tablet Composition

| | | % w/w | % w/w | % w/w | % w/w |
|---|---|---|---|---|---|
| | | (0% | (2.5% | (5% | (10% |
| Component | Function | CS) | CS) | CS | CS) |
| Rifaximin | Drug | 35.29 | 35.32 | 35.29 | 35.29 |
| HPMC-AS | Polymer | 35.29 | 35.32 | 35.29 | 35.29 |
| Pluronic | Wetting | 4.19 | 4.19 | 4.19 | 4.19 |
| F-127 | agent | | | | |
| Croscarmellose | Rate | 0.00 | 1.92 | 3.94 | 8.31 |
| Na [Extra- | controlling | | | | |
| granular] | | | | | |
| Avicel PH102 | Filler | 19.53 | 17.55 | 15.59 | 11.22 |
| Croscarmellose | Disintegrant | 5.00 | 5.00 | 5.00 | 5.00 |
| Na [Extra- | | | | | |
| granular] | | | | | |
| Cab-O-Sil | Glidant | 0.20 | 0.20 | 0.20 | 0.20 |
| Magnesium | Lubricant | 0.50 | 0.50 | 0.50 | 0.50 |
| Stearate | | | | | |
| | Total | 100 | 100 | 100 | 100 |

### Example 16—Clinical Data

The following clinical data was obtained using an immediate release (IR) and sustained extended release (SER) tablet composition comprising a 40 mg or 80 mg dose of rifaximin with the following components. See Table 38.

TABLE 38

| | | Theoretical Quantity (mg/tablet) | | | |
|---|---|---|---|---|---|
| | | 80 mg- | 80 mg- | 40 mg- | 80 mg- |
| Ingredient | Function | IR | SER | IR | IR |
| Rifaximin | Active | 80 | 80 | 40 | 40 |
| HPMC-AS | Polymer | 80 | 80 | 40 | 40 |
| Poloxamer 407 | Surfactant | 9.49 | 9.49 | 4.75 | 4.75 |
| Croscarmellose | Dissolution | 30.15 | 11.33 | 20.74 | 11.33 |
| sodium | enhancer | | | | |
| Microcrystalline | Filler | 25.28 | 44.10 | 119.43 | 128.84 |
| cellulose | | | | | |
| Colloidal silicon | Glidant | 0.45 | 0.45 | 0.45 | 0.45 |
| dioxide | | | | | |
| Magnesium stearate | Lubricant | 1.13 | 1.13 | 1.13 | 1.13 |
| (non-bovine) | | | | | |
| Opadry II Blue | Coating | 11.92 | 11.92 | 11.92 | 11.92 |
| 85F90614 | | | | | |
| (PVA coating) | | | | | |
| Purified Water | Solvent for | — | — | — | — |
| | coating | | | | |
| | solution | | | | |

| | | | | |
|---|---|---|---|---|
| Total Theoretical Weight | 238.42 | 238.42 | 238.42 | 238.42 |

Note:
“IIR” means Immediate Release; and “SER” means Sustained Extended Release.

A Phase 2, randomized, double-blind, placebo-controlled, parallel multicenter study evaluation of the efficacy (prevention of hospitalization for complications of liver cirrhosis or all-cause mortality in subjects with early decompensation) and safety of rifaximin SSD tablets in subjects with early decompensated liver cirrhosis was conducted. Subjects with documented ascites who had not previously experienced SBP, EVB, or HRS were enrolled in the study. Subjects completed a 1 to 21-day Screening Period, a 24-week Treatment Period, and a 2-week Follow-up Period. Approximately 420 subjects who successfully completed the Screening Period were randomized in a 1:1:1:1:1:1 allocation to 1 of 6 treatment groups and entered the Treatment Period. All treatments were administered once daily at bedtime. Assessments of efficacy and safety were performed during clinic visits at Day 1 (baseline), Weeks 2, 4, 8, 12, 16, 20, and 24 (End of Treatment [EOT]). All subjects completed an End of Study (EOS) visit at Week 26 (or early termination if applicable) for final safety assessments.

### Inclusion Criteria

A subject was eligible for inclusion in this study if he/she met all of the following criteria:

1. Subject was ≧18 years of age.
2. Subject was male or female.
Females of childbearing (reproductive) potential had to have a negative serum pregnancy test at Screening and had to agree to use an acceptable method of contraception throughout their participation in the study. Acceptable methods of contraception included double barrier methods (condom with spermicidal jelly or a diaphragm with spermicide), hormonal methods (eg, oral contraceptives, patches or medroxyprogesterone acetate), or an intrauterine device (IUD) with a documented failure rate of less than 1% per year. Abstinence or partner(s) with a vasectomy could be considered an acceptable method of contraception at the discretion of the investigator. Note: Females who had been surgically sterilized (eg, hysterectomy or bilateral tubal ligation) or who were postmenopausal (total cessation of menses for >1 year) were not considered “females of childbearing potential.”
3. Subject had a diagnosis of liver cirrhosis and documented ascites, either by imaging study or physical exam (Note: Subjects with Grade 1 ascites were permitted in the study), but had not yet experienced any of the following complications of cirrhosis:
EVB—clinically significant gastrointestinal bleed
SBP—greater than 250 polymorphonuclear (PMN) cells/mm³ and/or positive monomicrobial culture in the ascitic fluid
Renal failure in the presence of ascites—rise in the serum creatinine by 0.5 mg/dL (to greater than 1.5 mg/dL), with ascites documented on physical examination, imaging, and/or admitted on diuretics for the treatment of ascites
Development of medically refractory ascites.
   4. Subject had a MELD score of ≧12, a MELDNa score of ≧12, or a Child-Pugh B Classification (score=7-9).
   5. Subject was capable of understanding the requirements of the study, and was willing to comply with all study procedures.
   6. Subject understood the language of the informed consent form, and was capable and willing to sign the informed consent form.
   7. If applicable, subject had a close family member or other personal contact that could provide continuing oversight to the subject and was available to the subject during the conduct of the trial.

### Exclusion Criteria

A subject was not eligible for inclusion in this study if any of the following criteria applied:

1. Subject had a history of a major psychiatric disorder, including uncontrolled major depression or controlled or uncontrolled psychoses within the past 24 months prior to signing the informed consent (Diagnostic and Statistical Manual of Mental Disorders, 4th.) that, in the opinion of the investigator, would prevent completion of the study, interfere with analysis of study results, or negatively impact the subject's participation in the study.
2. Subject had history of alcohol abuse or substance abuse within the past 3 months prior to signing the informed consent (Diagnostic and Statistical Manual of Mental Disorders, 4th.).
3. Subject had documented primary sclerosing cholangitis (Note: subjects with primary biliary cirrhosis were allowed in the study).
4. Subject had undergone prophylactic variceal banding within 2 weeks of Screening or was scheduled to undergo prophylactic variceal banding during the study (Note: subjects with previous prophylactic variceal banding were allowed to participate in the study).
5. Subject had been diagnosed with an infection for which they are currently taking oral or parenteral antibiotics.
6. Subject had significant hypovolemia, or any electrolyte abnormality that could affect mental function (eg, serum sodium<125 mEq/L, serum calcium>10 mg/dL).
7. Subject had severe hypokalemia as defined by a serum potassium concentration<2.5 mEq/L.
8. Subject was anemic, as defined by a hemoglobin concentration of ≦8 g/dL.
9. Subject had renal insufficiency with a creatinine of ≧1.5 mg/dL.

Note: Laboratory tests related to Inclusion/Exclusion criteria could be repeated once, before considering subject as a Screening Failure (given all other Inclusion/Exclusion criteria are met/not met respectively) at the discretion of the Investigator.

10. Subject showed presence of intestinal obstruction or has inflammatory bowel disease.
11. Subject had Type 1 or Type 2 diabetes that was poorly controlled in the opinion of the investigator or had had an HbA1c>12% within the past 3 months prior to Screening or at the Screening visit.
12. Subject had a history of seizure disorders.
13. Subject had unstable cardiovascular or pulmonary disease, categorized by a worsening in the disease condition that required a change in treatment or medical care within 30 days of randomization.
14. Subject had an active malignancy within the last 5 years (exceptions: basal cell carcinomas of the skin, or if female, in situ cervical carcinoma that had been surgically excised).
15. Subject had hepatocellular carcinoma (HCC). Note: Alpha-fetoprotein (AFP) concentration was measured at Screening. If the AFP was greater than 200 ng/mL, the subject was excluded from participation in the study. If the AFP was above the upper limit of normal and ≦200 ng/mL, cross-sectional imaging or ultrasonography techniques had to be used to rule out HCC.
16. Subject had any condition or circumstance that adversely affected the subject or could cause noncompliance with treatment or visits, could affect the interpretation of clinical data, or could otherwise contraindicate the subject's participation in the study.
17. If female, subject was pregnant or at risk of pregnancy, or was lactating.
18. Known varicella, herpes zoster, or other severe viral infection within 6 weeks of randomization.
19. Known human immunodeficiency virus (HIV) infection.
20. Subject had a positive stool test for Yersinia enterocolitica, Campylobacter jejuni, Salmonella, Shigella, ovum and parasites, and/or Clostridium difficile.
NOTE: Results of stool tests had to be confirmed as negative prior to randomization.
21. Subject had a history of tuberculosis infection and/or had received treatment for a tuberculosis infection. If subject had a previous positive skin test for tuberculosis antigen then they were to have a current negative chest X-ray to be eligible and could not have received previous treatment.
22. Subject was an employee of the site that was directly involved in the management, administration, or support of this study or was an immediate family member of the same.
23. Subject had a history of hypersensitivity to rifaximin, rifampin, rifamycin antimicrobial agents, or any of the components of rifaximin SSD.
24. Subject used any investigational product or device, or participated in another research study within 30 days prior to randomization.
25. Subject had a documented overt HE episode (Conn score>2) that had not resolved within 30 days of Visit 1 (Screening).

### Treatments Administered

There were 6 treatment groups as listed below. The compositional components are presented above in Table 38. All treatments were to be administered orally qhs (at every bed time). The duration of the treatment was 24 weeks.

Treatment A: rifaximin SSD 40 mg qhs (IR tablet)

Treatment B: rifaximin SSD 80 mg qhs (IR tablet)

Treatment C: rifaximin SSD 40 mg qhs (SER tablet)

Treatment D: rifaximin SSD 80 mg qhs (SER tablet)

Treatment E: rifaximin SSD 80 mg qhs (IR tablet)+rifaximin 80 mg qhs (SER tablet)

Treatment F: Placebo qhs

### Primary Efficacy Endpoints

Over the 24-week treatment period, the primary efficacy endpoint for the study was time to:
All-cause mortality, or
Hospitalization that was associated with 1 of the following complications of liver disease:
   HE—altered mental status diagnosed as HE, and defined as an increase of the Conn score to Grade≧2 (ie, 0 or 1 to ≧2).
   EVB—occurrence of a clinically significant gastrointestinal bleed was defined as:
      Bleeding from an esophageal or gastric varix at the time of endoscopy, or
      The presence of large varices with blood evident in the stomach, and no other identifiable cause of bleeding observed during endoscopy.
      In addition, 1 or more of the following criteria had to be present:
         Drop in hemoglobin of greater than 2 g/dL over the first 48 hours post hospital admission,
         Transfusion requirement of 2 units of blood or more within 24 hours of hospital admission,
         A systolic blood pressure of less than 100 mm Hg, or
         Pulse rate greater than 100 beat/min at the time of admission.
            Note: Baveno IV criteria was also used to further define variceal bleeding episodes.
SBP—greater than 250 PMNcells/mm³ and/or positive monomicrobial culture in the ascitic fluid.
HRS was defined as:
   Progressive rise in serum creatinine (>1.5 mg/dL) with no improvement after at least 2 days with diuretic withdrawal and volume expansion with albumin;
   Absence of parenchymal kidney disease;
   Oliguria;
   Absence of shock; and
   No current or recent (within 3 months prior randomization) treatment with nephrotoxic drugs.

### Key Secondary Efficacy Endpoints

The key secondary efficacy endpoints of this study were overall hospitalization rate for each of the individual component of the primary endpoint or all-cause mortality over the 24-week treatment period.

### Other Secondary Endpoints

Other secondary endpoints of this study were the following:
Time to first hospitalization or all-cause mortality for each individual component of the primary endpoint.
All-cause hospitalization rate over the 24-week Treatment Period.
Liver cirrhosis mortality over the 24-week Treatment Period.
Time to development of medically refractory ascites, defined as ascites which could either no longer be effectively managed by:
   A low sodium diet and maximal doses of diuretics (up to 400 mg spironolactone and 160 mg furosemide per day), or
   Diuretics, due to the inability to tolerate side effects of maximal doses of diuretics.
Hospitalizations over the 24-week treatment period for all other infections.
Hospitalization as the result of Acute Kidney Injury (AKI) that was not attributable to HRS and was defined by a rapid reduction (over less than 48 hours) of kidney function as evidenced by:
   A rise in serum creatinine, (with either an absolute increase in serum creatinine of ≧0.3 mg/dL (≧26.4 μmol/L) or percentage increase in serum creatinine of ≧50%), and
   A reduction in urine output (defined as <0.5 ml/kg/hr for more than 6 hours).
Change in indices of Health Outcomes (Chronic Liver Disease Questionnaire (CLDQ), Gastrointestinal Symptom Rating Scale (GSRS), Caregiver Burden Inventory (CBI), Epworth Sleepiness Scale (ESS)) at Weeks 4, 8, 12, 16, 20 and 24.
Pharmacokinetics of rifaximin and 25-desacetyl rifaximin assessing effects on factors including hepatic impairment, renal impairment and concomitant medications.
The critical flicker frequency (CFF) was assessed for each subject. CFF was assessed using a specialized CFF instrument.
Changes from baseline in blood ammonia concentrations at Weeks 2, 4, 8, 12, 16, 20 and 24.
Change from baseline in MELD (Model for End-Stage Liver Disease) and MELDNa (model end stage liver disease sodium) score at Weeks 2, 4, 8, 12, 16, 20 and 24.
Change from baseline in Child-Pugh score at Weeks 2, 4, 8, 12, 16, 20 and 24.

### Drug Concentration Measurements

Rifaximin and metabolite concentration data was collected according to the Full Population PK Sampling design recommended in the FDA Guidance for Industry: Population Pharmacokinetics.

### Disposition of Subjects

A total of 518 subjects were randomized in the study, of which 408 (78.8%) completed the study:
64 subjects in the 40 mg qhs IR group,
63 subjects in the 80 mg qhs IR group,
68 subjects in the 40 mg qhs SER group,
68 subjects in the 40 mg qhs SER group, 7
2 subjects in the 80 mg qhs SER group,
66 subjects in the combined IR/SER group and
75 subjects in the placebo group.

In total, 109 (21.0%) subjects prematurely discontinued from the study, with the largest number of discontinuations observed in the 80 mg qhs IR group (30.4%). The most common reason of premature discontinuation reported in the study was “withdrawal by subject”; this accounted for the premature discontinuation of 44 (8.5%) of all subjects that were randomized. This was followed by “death” which accounted for the premature discontinuation of 21 (4.1%) of all randomized subjects. Of all treatment groups, the 80 mg qhs IR group experienced the most number of premature discontinuations from the study (28 subjects in total), with “withdrawal by subject” reported as the most common reason of premature discontinuation (n=9).

### Data Sets Analyzed

Two datasets were analyzed: ITT population and PP populations.
ITT population was defined as all randomized subjects who took at least 1 dose of study drug.
PP population was defined as all subjects in the ITT population with the exception of those who failed to meet inclusion criteria 3 or 4, or meet exclusion criterion 1.
Safety population included all randomized subjects who took at least 1 dose of study drug.

The analyses of baseline characteristics and efficacy were performed on the ITT population. The primary efficacy analyses were also performed on the PP population as a sensitivity analysis.

### Analysis of Efficacy

The primary efficacy endpoint was the time to all-cause mortality or hospitalization that was associated with 1 of the following complications of liver disease: HE, EVB, SBP, or HRS over the 24-week treatment period was performed on the ITT population.

The primary analysis of time to hospitalization for any of the liver cirrhosis complications or all-cause mortality specified for the primary endpoint utilized a log-rank test stratified by analysis region (2-sided test at a significance level of 0.05). Pairwise treatment group comparisons (each of the rifaximin SSD groups versus placebo) utilizing the log-rank test was also performed.

Additionally, Kaplan-Meier methods were used to estimate the proportion of subjects experiencing hospitalization for any of the liver cirrhosis complications or all-cause mortality on Days 28, 56, 84, 112, 140, and 168 for each treatment group.

Other analyses of the primary efficacy endpoint include sensitivity analyses (primary efficacy endpoint analyses using PP population) and prespecified subgroup analyses.

### Time to Hospitalization for any of the Liver Cirrhosis Complications or all-Cause Mortality

The primary efficacy endpoint was the time to all-cause mortality or hospitalization that was associated with 1 of the following complications of liver disease: HE, EVB, SBP, or HRS over the 24-week treatment period. Subjects who terminated early for reasons other than death were contacted approximately 24 weeks from randomization to determine if they experienced the primary endpoint. In the case of a subject's death, the subject's caregiver (if applicable) was contacted.

The time to hospitalization for any of the liver cirrhosis complications or all-cause mortality was defined as the duration between the date of first dose of the study drug and the date of first hospitalization for any of the liver cirrhosis complications or all-cause mortality.

Subjects who completed the entire 24-week treatment period without death or meeting the definition of liver cirrhosis complications of HE, EVB, SBP, or HRS were censored at the date of final visit (date of last contact). Subjects who prematurely discontinued before the end of the 24-week treatment period for reasons other than death were contacted monthly via a follow-up phone call for capture of cirrhosis complications, hospitalization, or death information. Subjects who did not meet the primary endpoint were censored at the date of last contact.

### Primary Efficacy Analysis

The primary analysis did not demonstrate an overall statistically significant difference in time to hospitalization for any of the liver cirrhosis complications or all-cause mortality up to 24 weeks in any group. The overall treatment comparison effect for any of the rifaximin SSD treatments versus placebo was not statistically significant (stratified log-rank p=0.8062) (Table 39). FIG. 88 presents a Kaplan-Meier estimate for the distribution of time to hospitalization for any of the liver cirrhosis complications by treatment group for the ITT population. Based on the Kaplan-Meier estimates, the SER 80 mg qhs treatment group presented with the highest survival rate of all treatment groups and the combined IR/SER treatment group had the lowest survival rate; however this effect was not statistically significant (log-rank p=0.2262). FIG. 89 presents a Kaplan-Meier estimate for the distribution of time to all-cause mortality by treatment group for the ITT population. Based on the Kaplan-Meier estimates, the placebo group presented with the highest survival rate followed by the SER 80 mg qhs treatment group and the combined IR/SER treatment group had the lowest survival rate; however this effect was not statistically significant (log-rank p=0.7573). FIG. 90 presents a Kaplan-Meier estimate for the distribution of time to hospitalization for any of the liver cirrhosis complications or all-cause mortality by treatment group for the ITT population. Based on the Kaplan-Meier estimates, the SER 80 mg qhs treatment group presented with the highest survival rate and the combined IR/SER treatment group had the lowest survival rate; this effect was statistically significant (log-rank p=0.0420).

### Supportive Analysis Based on the PP Population

The results on the primary efficacy analysis based on the PP population were not consistent with the pairwise comparisons based on the ITT population (Table 39). The primary analysis on the PP population demonstrated a statistically significant difference in the time to hospitalization for any of the liver cirrhosis complications or all-cause mortality up to 24 weeks that was in favor of the SER 80 mg qhs treatment group versus placebo (stratified log-rank p=0.0464). There were no other statistically significant pairwise comparisons observed between the remaining active treatment groups and placebo (Table 40). The overall treatment comparison effect for any of the rifaximin SSD treatments versus placebo was not statistically significant (stratified log-rank p=0.9879). FIG. 91 presents a Kaplan-Meier estimate for the distribution of time to hospitalization for any of the liver cirrhosis complications or all-cause mortality by treatment group for the PP population. Based on the Kaplan-Meier estimates, the SER 80 mg qhs treatment group presented with the highest survival rate and the combined IR/SER treatment group had the lowest survival rate; this effect was statistically significant (log-rank p=0.0182).

TABLE 39
Time to Hospitalization for any of the Liver Cirrhosis Complications
or All-cause Mortality up to 24 Weeks - ITT Population

| | | | Censored | | |
|---|---|---|---|---|---|
| | # of Subjects | # of Events | <Week 24 | Week 24¹ | p-value² |
| Overall Treatment Comparison³ | | | | | |
| Any Rifaximin SSD Treatment | 422 | 50 (11.8%) | 31 (7.3%) | 341 (80.8%) | 0.8062 |
| Placebo | 94 | 10 (10.6%) | 11 (11.7%) | 73 (77.7%) | |
| Pairwise Comparisons | | | | | |
| (versus Placebo)³ | | | | | |
| Treatment A: Rifaximin SSD | 78 | 7 (9.0%) | 5 (6.4%) | 66 (84.6%) | 0.6316 |
| 40 mg qhs (IR Tablet) | | | | | |
| Treatment B: Rifaximin SSD | 91 | 15 (16.5%) | 9 (9.9%) | 67 (73.6%) | 0.2283 |
| 80 mg qhs (IR Tablet) | | | | | |
| Treatment C: Rifaximin SSD | 84 | 9 (10.7%) | 7 (8.3%) | 68 (81.0%) | 0.9666 |
| 40 mg qhs (SER Tablet) | | | | | |
| Treatment D: Rifaximin SSD | 89 | 4 (4.5%) | 6 (6.7%) | 79 (88.8%) | 0.0991 |
| 80 mg qhs (SER Tablet) | | | | | |
| Treatment E: Rifaximin SSD | 80 | 15 (18.8%) | 4 (5.0%) | 61 (76.3%) | 0.1792 |
| 80 mg qhs (IR Tablet) and | | | | | |
| Rifaximin SSD 80 mg qhs | | | | | |
| (SER Tablet) | | | | | |

IR = immediate release; ITT = intent to treat; qhs = once daily at bedtime; SER = sustained extended release; SSD = solid soluble dispersion.
¹Number of subjects censored at Week 24 (subject did not experience an event and was enrolled in the study at Week 24).
²P-value was obtained using a stratified log-rank test.
³Stratified by analysis region (study centers are grouped within 2 regions, centers in the United States and centers in Russia)

TABLE 40
Time to Hospitalization for any of the Liver Cirrhosis Complications
or All-cause Mortality up to 24 Weeks - PP Population

| | | | Censored | | |
|---|---|---|---|---|---|
| | # of Subjects | # of Events | <Week 24 | Week 24² | p-value³ |
| Overall Treatment Comparison⁴ | | | | | |
| Any Rifaximin SSD Treatment | 403 | 46 (11.4%) | 31 (7.7%) | 326 (80.9%) | 0.9879 |
| Placebo | 90 | 10 (11.1%) | 10 (11.1%) | 70 (77.8%) | |
| Pairwise Comparisons | | | | | |
| (versus Placebo)⁴ | | | | | |
| Treatment A: Rifaximin SSD | 72 | 5 (6.9%) | 5 (6.9%) | 62 (86.1%) | 0.3116 |
| 40 mg qhs (IR Tablet) | | | | | |
| Treatment B: Rifaximin SSD | 88 | 15 (17.0%) | 9 (10.2%) | 64 (72.7%) | 0.2247 |
| 80 mg qhs (IR Tablet) | | | | | |
| Treatment C: Rifaximin SSD | 81 | 9 (11.1%) | 7 (8.6%) | 65 (80.2%) | 0.9641 |
| 40 mg qhs (SER Tablet) | | | | | |
| Treatment D: Rifaximin SSD | 85 | 3 (3.5%) | 6 (7.1%) | 76 (89.4%) | 0.0464 |
| 80 mg qhs (SER Tablet) | | | | | |
| Treatment E: Rifaximin SSD | 77 | 14 (18.2%) | 4 (5.2%) | 59 (76.6%) | 0.2523 |
| 80 mg qhs (IR Tablet) and | | | | | |
| Rifaximin SSD 80 mg qhs | | | | | |
| (SER Tablet) | | | | | |

IR = immediate release; ITT = intent to treat; PP = per protocol; qhs = once daily at bedtime; SER = sustained extended release; SSD = solid soluble dispersion.
¹All subjects in the ITT population except those that failed inclusion criteria 3, 4 or met exclusion criterion 1.
²Number of subjects censored at Week 24 (subject did not experience an event and was enrolled in the study at Week 24.
³P-value was obtained using a stratified log-rank test.
⁴Stratified by analysis region (study centers are grouped within 2 regions, centers in the United States and centers in Russia).

### Prespecified Subgroup Analyses of the Primary Efficacy Endpoint

### Baseline MELD Category

The influence of a subject's baseline MELD category on the primary efficacy analysis was evaluated. Baseline MELD subgroups were categorized as MELD scores of ≦10, 11 to 18, 19 to 24, or ≧25. None of the pairwise comparisons versus placebo were statistically significant in any of the subgroups. The overall treatment comparison effect for any of the rifaximin SSD treatment versus placebo was not statistically significant (MELD score of ≦10: stratified log-rank p=0.8486; MELD score: 11 to 18 stratified log-rank p=0.7937; MELD score of 19 to 24: stratified log-rank p=0.3154; and MELD score of ≧25: stratified log-rank p=not applicable [1 event out of 1 subject]).

### Baseline MELDNa Category

The influence of a subject's baseline MELDNa category on the primary efficacy analysis was evaluated. Baseline MELDNa subgroups were categorized as MELDNa scores of ≦10, 11 to 18, 19 to 24, or ≧25. None of the pairwise comparisons versus placebo were statistically significant in any of the subgroups. The overall treatment comparison effect for any of the rifaximin SSD treatment versus placebo was not statistically significant (MELDNa score of ≦10: stratified log-rank p=0.3200; MELDNa score: 11 to 18 stratified log-rank p=0.9368; MELDNa score of 19 to 24: stratified log-rank p=0.2608; and MELDNa score of ≧25: stratified log-rank p=not applicable [3 events out of 4 subjects]).

### Baseline Child-Pugh Classification

The influence of a subject's baseline Child-Pugh classification on the primary efficacy analysis was evaluated. The baseline Child-Pugh classification subgroups were categorized as Class A, Class B, or Class C. None of the pairwise treatment comparisons versus placebo were statistically significant in any of the subgroups. The overall treatment comparison effect for any of the rifaximin SSD treatments versus placebo was not statistically significant (Class A: stratified log-rank p=not applicable [zero events]; Class B: stratified log-rank p=0.7942 and Class C: stratified log-rank p=0.9516).

### Baseline Conn Score

The influence of a subject's baseline Conn score on the primary efficacy analysis was evaluated. Baseline Conn score subgroups were categorized as 0, 1, or 2. Table 41 presents the analysis of the primary efficacy endpoint by baseline Conn score. Consistent with the results of the PP population, a statistically significant difference in the time to hospitalization for any of the liver cirrhosis complications or all-cause mortality was observed within the Conn score subgroup 0 and was in favor of the SER 80 mg qhs treatment group versus placebo (stratified log-rank p=0.0460). This statistical significance was not evident within the Conn score subgroups 1 or 2 (although, subgroup 2 had 1 event out of 2 subjects).

The overall treatment comparison effect for any of the rifaximin SSD treatments versus placebo was not statistically significant for any subgroup (Conn score 0: stratified log-rank p=0.8915; Conn score 1: stratified log-rank p=0.8251; Conn score 2: not applicable [1 event out of 2 subjects]).

TABLE 41
Analysis of Primary Efficacy Endpoint: Time to Hospitalization for
any of the Liver Cirrhosis Complications or All-cause Mortality
by Baseline Conn Score up to 24 Weeks (Day 170) - ITT Population

| | | | Censored | | |
|---|---|---|---|---|---|
| | # of Subjects | # of Events | <Week 24 | Week 24¹ | p-value² |
| Conn Score: 0 | | | | | |
| Overall Treatment Comparison³ | | | | | |
| Any Rifaximin SSD Treatment | 260 | 34 (13.1%) | 20 (7.7%) | 206 (79.2%) | 0.8915 |
| Placebo | 57 | 7 (12.3%) | 6 (10.5%) | 44 (77.2%) | |
| Pairwise Comparisons | | | | | |
| (versus Placebo)³ | | | | | |
| Treatment A: Rifaximin SSD | 48 | 7 (14.6%) | 3 (6.3%) | 38 (79.2%) | 0.7477 |
| 40 mg qhs (IR Tablet) | | | | | |
| Treatment B: Rifaximin SSD | 55 | 11 (20.0%) | 7 (12.7%) | 37 (67.3%) | 0.2297 |
| 80 mg qhs (IR Tablet) | | | | | |
| Treatment C: Rifaximin SSD | 53 | 4 (7.5%) | 5 (9.4%) | 44 (83.0%) | 0.4007 |
| 40 mg qhs (SER Tablet) | | | | | |
| Treatment D: Rifaximin SSD | 48 | 1 (2.1%) | 4 (8.3%) | 43 (89.6%) | 0.0460 |
| 80 mg qhs (SER Tablet) | | | | | |
| Treatment E: Rifaximin SSD | 56 | 11 (19.6%) | 1 (1.8%) | 44 (78.6%) | 0.3340 |
| 80 mg qhs (IR Tablet) and | | | | | |
| Rifaximin SSD 80 mg qhs | | | | | |
| (SER Tablet) | | | | | |
| Conn Score: 1 | | | | | |
| Overall Treatment Comparison³ | | | | | |
| Any Rifaximin SSD Treatment | 160 | 15 (9.4%) | 11 (6.9%) | 134 (83.8%) | 0.8251 |
| Placebo | 37 | 3 (8.1%) | 5 (13.5%) | 29 (78.4%) | |
| Pairwise Comparisons | | | | | |
| (versus Placebo)³ | | | | | |
| Treatment A: Rifaximin SSD | 30 | 0 | 2 (6.7%) | 28 (93.3%) | 0.0941 |
| 40 mg qhs (IR Tablet) | | | | | |
| Treatment B: Rifaximin SSD | 36 | 4 (11.1%) | 2 (5.6%) | 30 (83.3%) | 0.7015 |
| 80 mg qhs (IR Tablet) | | | | | |
| Conn Score 1 Pairwise Comparisons | | | | | |
| (versus Placebo)³, Continued | | | | | |
| Treatment C: Rifaximin SSD | 31 | 5 (16.1%) | 2 (6.5%) | 24 (77.4%) | 0.3467 |
| 40 mg qhs (SER Tablet) | | | | | |
| Treatment D: Rifaximin SSD | 39 | 2 (5.1%) | 2 (5.1%) | 35 (89.7%) | 0.5204 |
| 80 mg qhs (SER Tablet) | | | | | |
| Treatment E: Rifaximin SSD | 24 | 4 (16.7%) | 3 (12.5%) | 17 (70.8%) | 0.3075 |
| 80 mg qhs (IR Tablet) and | | | | | |
| Rifaximin SSD 80 mg qhs (SER | | | | | |
| Tablet) | | | | | |
| Conn Score: 2 | | | | | |
| Overall Treatment Comparison³ | | | | | |
| Any Rifaximin SSD Treatment | 2 | 1 (50.0%) | 0 | 1 (50.0%) | |
| Pairwise Comparisons | | | | | |
| (versus Placebo)³ | | | | | |
| Treatment D: Rifaximin SSD | 2 | 1 (50.0%) | 0 | 1 (50.0%) | |
| 80 mg qhs (SER Tablet) | | | | | |

IR = immediate release; ITT = intent to treat; qhs = once daily at bedtime; SER = sustained extended release; SSD = solid soluble dispersion.
¹Number of subjects censored at Week 24 (subject did not experience an event and was enrolled in the study at Week 24).
²P-value was obtained using a stratified log-rank test.
³Stratified by analysis region (study centers are grouped within 2 regions, centers in the United States and centers in Russia).

### Time Since First Diagnosis of Liver Cirrhosis

The influence of a subject's time since first diagnosis of liver cirrhosis on the primary efficacy analysis was evaluated. The time since first diagnosis of liver cirrhosis subgroups were categorized as <947 days or ≧947 days. Table 42 presents the analysis of the primary efficacy endpoint by time since first diagnoses of liver cirrhosis. A near statistically significant difference in the time to hospitalization for any of the liver cirrhosis complications or all-cause mortality was observed within ≧947 days subgroup and, like the PP and baseline Conn score 0 populations, was in favor of the SER 80 mg qhs treatment group versus placebo (stratified log-rank p=0.0517). The overall treatment comparison effect for any of the rifaximin SSD treatment versus placebo was not statistically significant (time since first diagnosis of liver cirrhosis: <947 days stratified log-rank p=0.3961; time since first diagnosis of liver cirrhosis: ≧947 days stratified log-rank p=0.5689).

TABLE 42
Analysis of Primary Efficacy Endpoint: Time to Hospitalization for any of the
Liver Cirrhosis Complications or All-cause Mortality by Categorized Time Since
First Diagnosis of Liver Cirrhosis up to 24 Weeks (Day 170) - ITT Population

| | | | Censored | | |
|---|---|---|---|---|---|
| | # of Subjects | # of Events | <Week 24 | Week 24¹ | p-value² |
| <947 Days | | | | | |
| Overall Treatment Comparison³ | | | | | |
| Any Rifaximin SSD Treatment | 206 | 32 (15.5%) | 14 (6.8%) | 160 (77.7%) | 0.3961 |
| Placebo | 50 | 5 (10.0%) | 7 (14.0%) | 38 (76.0%) | |
| Pairwise Comparisons | | | | | |
| (versus Placebo)³ | | | | | |
| Treatment A: Rifaximin SSD | 43 | 3 (7.0%) | 3 (7.0%) | 37 (86.0%) | 0.4929 |
| 40 mg qhs (IR Tablet) | | | | | |
| Treatment B: Rifaximin SSD | 41 | 8 (19.5%) | 7 (17.1%) | 26 (63.4%) | 0.2329 |
| 80 mg qhs (IR Tablet) | | | | | |
| Treatment C: Rifaximin SSD | 46 | 8 (17.4%) | 2 (4.3%) | 36 (78.3%) | 0.3436 |
| 40 mg qhs (SER Tablet) | | | | | |
| Treatment D: Rifaximin SSD | 34 | 3 (8.8%) | 1 (2.9%) | 30 (88.2%) | 0.7582 |
| 80 mg qhs (SER Tablet) | | | | | |
| Treatment E: Rifaximin SSD | 42 | 10 (23.8%) | 1 (2.4%) | 31 (73.8%) | 0.1237 |
| 80 mg qhs (IR Tablet) and | | | | | |
| Rifaximin SSD 80 mg qhs | | | | | |
| (SER Tablet) | | | | | |
| ≧947 Days | | | | | |
| Overall Treatment Comparison³ | | | | | |
| Any Rifaximin SSD Treatment | 215 | 18 (8.4%) | 16 (7.4%) | 181 (84.2%) | 0.5689 |
| Placebo | 44 | 5 (11.4%) | 4 (9.1%) | 35 (79.5%) | |
| Pairwise Comparisons | | | | | |
| (versus Placebo)³ | | | | | |
| Treatment A: Rifaximin SSD | 34 | 4 (11.8%) | 1 (2.9%) | 29 (85.3%) | 0.9598 |
| 40 mg qhs (IR Tablet) | | | | | |
| Treatment B: Rifaximin SSD | 50 | 7 (14.0%) | 2 (4.0%) | 41 (82.0%) | 0.6094 |
| 80 mg qhs (IR Tablet) | | | | | |
| Treatment C: Rifaximin SSD | 38 | 1 (2.6%) | 5 (13.2%) | 32 (84.2%) | 0.1523 |
| 40 mg qhs (SER Tablet) | | | | | |
| Treatment D: Rifaximin SSD | 55 | 1 (1.8%) | 5 (9.1%) | 49 (89.1%) | 0.0517 |
| 80 mg qhs (SER Tablet) | | | | | |
| Treatment E: Rifaximin SSD | 38 | 5 (13.2%) | 3 (7.9%) | 30 (78.9%) | 0.8519 |
| 80 mg qhs (IR Tablet) and | | | | | |
| Rifaximin SSD 80 mg qhs | | | | | |
| (SER Tablet) | | | | | |

IR = immediate release; ITT = intent to treat; qhs = once daily at bedtime; SER = sustained extended release; SSD = solid soluble dispersion.
¹Number of subjects censored at Week 24 (subject did not experience an event and was enrolled in the study at Week 24).
²P-value was obtained using a stratified log-rank test.
³Stratified by analysis region (study centers are grouped within 2 regions, centers in the United States and centers in Russia).

### Time to Development of Medically Refractory Ascites up to Week 24 (Day 170)

Analysis of the time to development of medically refractory ascites up to Week 24 (Day 170) is presented in Table 43.

A statistically significant difference in time to development of medically refractory ascites up to 24 Week was observed in favor of the IR 40 mg qhs treatment group versus placebo (stratified log-rank p=0.0308) and in favor of the SER 40 mg qhs treatment group versus placebo (stratified log-rank p=0.0202). No other pairwise treatment comparisons versus placebo were statistically significant. The overall treatment comparison for any of the rifaximin SSD treatments versus placebo was not statistically significant.

TABLE 43
Analysis of Secondary Efficacy Endpoint: Time to Development of Medically
Refractory Ascites up to 24 Week (Day 170) - ITT Population

| | | | Censored | | |
|---|---|---|---|---|---|
| | # of Subjects | # of Events | <Week 24 | Week 24¹ | p-value² |
| Overall Treatment | | | | | |
| Comparison³ | | | | | |
| Any Rifaximin SSD | 422 | 16 (3.8%) | 51 (12.1%) | 355 (84.1%) | 0.0601 |
| Treatment | | | | | |
| Placebo | 94 | 0 | 13 (13.8%) | 81 (86.2%) | |
| Pairwise Comparisons | | | | | |
| (versus Placebo)³ | | | | | |
| Treatment A: Rifaximin | 78 | 4 (5.1%) | 8 (10.3%) | 66 (84.6%) | 0.0308 |
| SSD 40 mg qhs | | | | | |
| (Immediate Release [IR] | | | | | |
| Tablet) | | | | | |
| Treatment B: Rifaximin | 91 | 3 (3.3%) | 18 (19.8%) | 70 (76.9%) | 0.0721 |
| SSD 80 mg qhs | | | | | |
| (Immediate Release [IR] | | | | | |
| Tablet) | | | | | |
| Treatment C: Rifaximin | 84 | 5 (6.0%) | 9 (10.7%) | 70 (83.3%) | 0.0202 |
| SSD 40 mg qhs | | | | | |
| (Sustained Extended | | | | | |
| Release [SER] Tablet) | | | | | |
| Treatment D: Rifaximin | 89 | 2 (2.2%) | 8 (9.0%) | 79 (88.8%) | 0.1508 |
| SSD 80 mg qhs | | | | | |
| (Sustained Extended | | | | | |
| Release [SER] Tablet) | | | | | |
| Treatment E: Rifaximin | 80 | 2 (2.5%) | 8 (10.0%) | 70 (87.5%) | 0.1319 |
| SSD 80 mg qhs | | | | | |
| (IR Tablet) and Rifaximin | | | | | |
| SSD 80 mg qhs (SER | | | | | |
| Tablet) | | | | | |

IR = immediate release; ITT = intent to treat; qhs = once daily at bedtime; SER = sustained extended release; SSD = solid soluble dispersion.
¹Number of subjects censored at Week 24 (subject did not experience an event and was enrolled in the study at Week 24).
²P-value was obtained using a stratified log-rank test.
³Stratified by analysis region (study centers are grouped within 2 regions, centers in the United States and centers in Russia).

### Efficacy Conclusions

Based on Kaplan Meier estimates of distribution of time to hospitalization for any of the liver cirrhosis complications or all-cause mortality up to 24 weeks, there was a statistically significant effect in favor of the SER 80 mg qhs and combined IR/SER qhs treatment groups having the highest and lowest survival rates, respectively.

The primary analysis on the PP population did demonstrate a statistically significant difference in the time to hospitalization for any of the liver cirrhosis complications or all-cause mortality up to 24 weeks that was in favor of the SER 80 mg qhs treatment group versus placebo. Kaplan Meier estimates of distribution of time to hospitalization for any of the liver cirrhosis complications or all-cause mortality up to 24 weeks were also statistically significant in favor of the SER 80 mg qhs and combined IR/SER qhs treatment groups having the highest and lowest survival rates, respectively.

In the secondary analysis, there was a statistically significant difference in time to development of medical refractory ascites up to Week 24 in favor of the IR 40 mg qhs and SER 40 mg qhs treatment groups versus placebo. There was a statistically significant effect for change from baseline in ESS total score was statistically significant treatment versus placebo effect was observed at Week 4 at the 25th percentile for baseline (p<0.0001), with the IR 40 mg qhs treatment group presenting with the greatest decrease from baseline.

These studies show, for the primary analysis, overall time to hospitalization for any of the liver cirrhosis complications or all-cause mortality up to 24 weeks was in favor of the SER 80 mg qhs treatment group versus placebo. In the secondary analysis, statistically significant favorable effects were observed most consistently in the IR 40 mg qhs treatment group as well as occurrences in the combined IR/SER qhs and SER 40 mg qhs treatment groups.

What is claimed is:

1. A pharmaceutical composition comprising
from about 33 wt % to about 35 wt % rifaximin;
from about 33 wt % to about 35 wt % HPMC-AS;
from about 3 wt % to about 5 wt % poloxamer 407;
from about 4 wt % to about 14 wt % croscarmellose sodium;
from about 10 wt % to about 19 wt % microcrystalline cellulose;
from about 0.15 wt % to about 0.25 wt % colloidal silicon dioxide; and
from about 0.45 wt % to about 0.55 wt % magnesium stearate.

2. The pharmaceutical composition of claim 1, wherein the croscarmellose sodium is present in an amount of from about 12 wt % to about 14 wt %.

3. The pharmaceutical composition of claim 1, wherein the croscarmellose sodium is present in an amount of about 13%.

4. The pharmaceutical composition of claim 1, wherein the microcrystalline cellulose is present in an amount from about 10 wt % to about 12 wt %.

5. The pharmaceutical composition of claim 1, wherein the microcrystalline cellulose is present in an amount of about 11 wt %.

6. The pharmaceutical composition of claim 1, wherein the croscarmellose sodium is present in an amount from about 4 wt % to about 6 wt %.

7. The pharmaceutical composition of claim 1, wherein the croscarmellose sodium is present in an amount of about 5 wt %

8. The pharmaceutical composition of claim 1, wherein the microcrystalline cellulose is present in an amount from about 17 wt % to about 19 wt %.

9. The pharmaceutical composition of claim 1, wherein the microcrystalline cellulose is present in an amount of about 18 wt %.

10. The pharmaceutical composition of claim 1, wherein the poloxamer 407 is present in an amount of about 4%.

11. The pharmaceutical composition of claim 1, wherein the colloidal silicon dioxide is present in an amount of about 0.20 wt %.

12. The pharmaceutical composition of claim 1, wherein the magnesium stearate is present in an amount of about 0.50 wt %.

13. The pharmaceutical composition of claim 1, wherein the rifaximin is present in an amount of about 34%.

14. The pharmaceutical composition of claim 1, wherein the HPMC-AS is present in an amount of about 34%.

15. The pharmaceutical composition of claim 1, wherein the total amount of rifaximin is about 80 mg.

16. A pharmaceutical composition comprising
from about 16 wt % to about 18 wt % rifaximin;
from about 16 wt % to about 18 wt % HPMC-AS;
from about 1 wt % to about 2 wt % poloxamer 407;
from about 4 wt % to about 10 wt % croscarmellose sodium;
from about 49 wt % to about 55 wt % microcrystalline cellulose;
from about 0.15 wt % to about 0.25 wt % colloidal silicon dioxide; and
from about 0.45 wt % to about 0.55 wt % magnesium stearate.

17. The pharmaceutical composition of claim 16, wherein the croscarmellose sodium is present in an amount from about 8 wt % to about 10 wt %.

18. The pharmaceutical composition of claim 16, wherein the croscarmellose sodium is present in an amount of about 9 wt %.

19. The pharmaceutical composition of claim 16, wherein the microcrystalline cellulose is present in an amount from about 49 wt % to about 51 wt %.

20. The pharmaceutical composition of claim 16, wherein the microcrystalline cellulose is present in an amount of about 51 wt %.

21. The pharmaceutical composition of claim 16, wherein the croscarmellose sodium is present in an amount from about 4 wt % to about 6 wt %.

22. The pharmaceutical composition of claim 16, wherein the croscarmellose sodium is present in an amount of about 5 wt %

23. The pharmaceutical composition of claim 16, wherein the microcrystalline cellulose is present in an amount from about 53 wt % to about 55 wt %.

24. The pharmaceutical composition of claim 16, wherein the microcrystalline cellulose is present in an amount of about 54 wt %.

25. The pharmaceutical composition of claim 16, wherein colloidal silicon dioxide is present in an amount of about 0.20 wt %.

26. The pharmaceutical composition of claim 16, wherein the magnesium stearate is present in an amount of about 0.50 wt %.

27. The pharmaceutical composition of claim 16, wherein the rifaximin is present in an amount of about 17 wt %.

28. The pharmaceutical composition of claim 16, wherein the HMPC-AS is present in an amount of about 17 wt %.

29. The pharmaceutical composition of claim 16, wherein the total amount of rifaximin is 40 mg.

30. The pharmaceutical composition of claim 1, wherein the composition is in the form of a tablet.

31. The pharmaceutical composition of claim 16, wherein the composition is in the form of a tablet.

32. The pharmaceutical composition of claim 30, wherein the composition is immediate release or sustained extended release.

33. The pharmaceutical composition of claim 31, wherein the composition is immediate release or sustained extended release.

34. A method of reducing the time to hospitalization associated with a complication of liver disease in a subject, comprising administering to the subject a composition of any claim 1.

35. The method of claim 34, wherein the complication of liver disease is selected from one or more of HE, EVB, SBP, and HRS.

36. A method of reducing the time to hospitalization associated with a complication of liver disease in a subject, comprising administering to the subject a composition of any claim 16.

37. The method of claim 36, wherein the complication of liver disease is selected from one or more of HE, EVB, SBP, and HRS.

38. A method of reducing the time to development of refractory ascites in a subject, comprising administering to the subject a composition of claim 1.

39. A method of reducing the time to development of refractory ascites in a subject, comprising administering to the subject a composition of claim 16.
